(19) United States
(12) Patent Application Publication
(10) Pub. No.: US 2003/0095958 A1
(43) Pub. Date: May 22, 2003

(54) Inhibitors of bace

(76) Inventors: Govinda R. Bhisetti, Lexington, MA (US); Jeffrey O. Saunders, Acton, MA (US); Mark A. Murcko, Holliston, MA (US); Christopher A. Lepre, Concord, MA (US); Shawn D. Britt, Andover, MA (US); Jon H. Come, Cambridge, MA (US); David D. Deininger, Arlington, MA (US); Tianshang Wang, Concord, MA (US)

Correspondence Address: VERTEX PHARMACEUTICALS INCORPORATED, 130 Waverly Street, Cambridge, MA, 02130-4646, US

(21) Appl. No.: 10/136,576

(22) Filed: Apr. 29, 2002

Related U.S. Application Data
(60) Provisional application No. 60/287,169, filed on Apr. 27, 2001.
(60) Provisional application No. 60/301,049, filed on Jun. 26, 2001.
(60) Provisional application No. 60/342,263, filed on Dec. 18, 2001.

(51) Int. Cl. A61K 38/43; C12N 9/99; C07H 21/04; C12P 21/02; C12N 5/06

(52) U.S. Cl. 424/94.1; 435/184; 435/69.2; 435/320.1; 435/325; 536/23.2

(57) ABSTRACT

The present invention relates to inhibitors of aspartic proteinases, particularly, BACE. The present invention also relates to compositions thereof and methods therewith for inhibiting BACE activity in a mammal, and for treating Alzheimer's Disease and other BACE-mediated diseases.

### TECHNICAL FIELD OF THE INVENTION

The present invention relates to inhibitors of aspartic proteinases, particularly, BACE. The present invention also relates to compositions thereof and methods therewith for inhibiting BACE activity in a mammal, and for treating Alzheimer's Disease and other BACE-mediated diseases.

### BACKGROUND OF THE INVENTION

Aspartic proteinases are found in a variety of pathways in different eukaryotic organisms, including mammals, viral, fungal and parasitic organisms. For example, BACE-1 (hereinafter “BACE”), as discussed below, has been implicated in the pathogenesis of Alzheimer's Disease (“AD”). BACE-2, an aspartic proteinase with high homology to BACE, is a glycosylated transmembrane protein with potentially similar disease implications as BACE. Renin, a well-known aspartic proteinase, is part of a critical signaling pathway that creates balance in blood pressure. See, e.g., Tamura K. et al., “Recent Advances in the Study of Renin and Angiotensinogen genes: from molecules to the whole body,” Hypertens. Res., 18(1) pp. 7-18 (1995). Renin has been implicated in hypertension and other cardiovascular conditions. Napsin-A and Napsin-B are closely related aspartic proteinases. Napsin-A is expressed in lung and kidney tissue and has been implicated in lung adenocarcinoma. Chuman, Y. et al., “Napsin A, a member of the aspartic protease family, is abundantly expressed in normal lung and kidney tissue and also expressed in lung adenocarcinomas,” FEBS Lett., 462(1-2): pp. 1.29-34 (1999). Cathepsin-D, a lysosomal aspartic proteinase, is expressed in all tissues and is implicated in protein catabolism, antigen processing, degenerative diseases and breast cancer progression. See, e.g., Erickson, J. W., et al., “Structure of human Cathepsin D: comparison of inhibitor binding and subdomain displacement with other aspartic proteinases,” Adv. Exp. Med. Biol., 362, pp. 181-192 (1995). Cathepsin-E, a non-lysosomal aspartic proteinase, may play a role in proteolytic degradation of antigen, which is a major regulatory step in the activation of a T-lymphocyte response. Bennet, K. et al., “Antigen processing for presentation by Class II major histocompatibility complex requires cleavage by cathepsin E,” Eur. J. Immunol., 22(6), pp 1519-24 (1992). Pepsinogen-A and Pepsinogen-C, both aspartic proteinase secreted in the stomach, are involved in the digestion of proteins in the stomach. Richter, C. et al., “Mechanism of activation of the gastric aspartic proteinases: pepsinogen, progastricin and prochymosin,” Biochem. J., 335, pp. 481-90 (1998). Pepsinogen-C is also found in the prostate and the seminal fluid.

Recently, BACE has received significant attention due to its implication in the pathogenesis of AD. Yi Luo et al., “Mice deficient in BACE1, the Alzheimer's β-secretase, have normal phenotype and abolished β-amyloid generation,” Nature Neuroscience, 4(3), pp. 231-232 (2001). AD is the most common cause of dementia in western industrialized countries. Individuals who develop AD experience progressive loss of memory and other cognitive functions that compromise their ability to work, interact socially, and care for themselves. These impairments are associated with widespread damage to several classes of neurons and different neurotransmitter systems in the brain. The symptoms and pathology of AD are progressive. People with AD eventually become dependent on others for all aspects of their care.

Currently available treatments provide limited benefit to people with Alzheimer's Disease. Drugs that augment cholinergic neurotransmission by inhibiting the enzyme acetylcholinesterase have been approved for use in humans. These drugs have been shown to improve cognitive function modestly but not to slow underlying disease progression. A major need therefore exists for treatments that modify underlying progression of AD.

The pathological hallmarks of AD are senile plaques (SPs) and neurofibrillary tangles (NFTs). Senile plaques comprise extracellular aggregates of Aβ protein, dystrophic neurites, activated microglia, and reactive astrocytes. Aβ is 40-42-residue endoproteolytic fragment of the amyloid precursor protein (“APP”). The cause of AD has not been established, but a growing body of data indicates that Aβ plays a central role in disease pathogenesis.

Aβ is produced in vivo following proteolytic cleavage of the membrane-anchored APP at the β site by β-secretase, followed by cleavage at the γ site by γ-secretase. The γ site lies on the lumenal side of the membrane. The γ site lies in the transmembrane domain and is more variable. γ Cleavage at residue 711 yields Aγ₁₋₄₀. γ Cleavage at residue 713 yields Aγ₁₋₄₂. Cleavage at the β site is the rate-limiting step in production of Aβ in vivo. Tang et al., “Structure of the Protease Domain of Memapsin 2 (β-Secretase) Complexed with Inhibitor,” Science, v. 290, pp. 150-53 (2000); Cai et al., “BACE1 is the major β-secretase for generation of Aβ peptides by neurons,” Nature Neuroscience, 4(3), pp. 233-234 (2001).

The enzyme responsible for β cleavage has been purified, and the gene encoding the protein responsible for this activity sequenced and cloned [EP 855,444; WO 00/47618]. Variously designated as β secretase, β amyloid converting enzyme (“BACE”), Asp 2, and memapsin 2, this enzyme is an aspartic proteinase. BACE is expressed as a 501 amino acid pro-polypeptide containing an N-terminal signal sequence and pro region that is cleaved post-translationally. BACE also contains a C-terminal trans-membrane domain and exists in cells as a membrane-bound protein.

Known peptidyl inhibitors of BACE are not readily suitable for therapy because, typically, they do not cross the blood-brain barrier. Thus, there is a need for peptidyl inhibitors of BACE that readily cross the blood-brain barrier. There are no reported non-peptidyl inhibitors of BACE. Thus, there is a need for non-peptidyl BACE inhibitors and compositions thereof. There is also a need for inhibitors of other aspartic proteinases and methods for designing such inhibitors of aspartic proteinases.

There is also a need for compounds and compositions useful in treating BACE-mediated diseases. There is also a need for methods for treating diseases such as Alzheimer's Disease and related neurological disorders.

### SUMMARY OF THE INVENTION

It is an object of the present invention to provide an inhibitor of BACE having the following structural features:

(a) HB-1;

(b) HPB-4;

and at least one of the following (c) and (d):

(c) HPB-2; and

(d) HPB-3,

wherein:

HB-1 is a first hydrogen-bonding moiety capable of forming up to four hydrogen bonds with the carboxylate oxygen atoms of Asp-228 and Asp-32 of BACE.

HPB-2 is a second hydrophobic moiety capable of associating with substantially all residues in the Flap binding pocket of BACE;

HPB-3 is a third hydrophobic moiety capable of associating with substantially all residues in the P2′ binding pocket of BACE;

HPB-4 is a fourth hydrophobic moiety capable of inducing favorable interactions with the phenyl ring of at least two of Tyr-71, Phe-108 and Trp-76 of BACE.

It is an object of the present invention to provide a method of inhibiting BACE activity in a mammal, comprising the step of administering to said mammal a compound of formula IA:

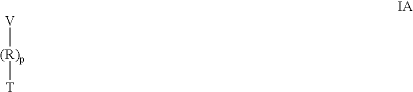

or a pharmaceutically acceptable salt thereof, wherein:

V is a 3-4 membered acyclic group or a 5-7 membered, fully or partially saturated cyclic group;

wherein V comprises a first moiety selected from NH, CH—OH, or a CH—NH₂, and a second moiety selected from carbon, CH, or N;

wherein said first moiety and said second moiety in V are non-adjacent; and

V is attached to R through said second moiety;

wherein V is optionally substituted with R¹⁰;

R is a suitable linker;

is 0 or 1;

R¹⁰ is P1-R1-P2-R2-W;

T is a five to eleven membered monocyclic or bicyclic, aromatic or non-aromatic ring having zero to three heteroatoms independently selected from O, S, N or NH, wherein T has at least one R¹⁰ substituent and up to three more substituents selected from R¹⁰ or J;

J is halogen, —R′, —OR′, —NO₂, —CN, —CF₃, —OCF₃, oxo, 1,2-methylenedioxy, —N(R′)₂, —SR′, —S(O)R′, —S(O)N(R′)₂, —SO₂R′, —C(O)R′, —CO₂R′, —C(O)N(R′)₂, —N(R′)C(O)R′, —N(R′)C(O)OR′, —N(R′)C(O)N(R′)₂, or —OC(O)N(R′)₂, wherein R′ is independently selected from hydrogen, aliphatic, heterocyclyl, heterocycly-alkyl, aryl, aralkyl, heteroaryl, or heteroaralkyl;

wherein R′ is optionally substituted with up to 3 substituents selected independently from —R¹¹, —OR¹¹, —NO₂, —CN, —CF₃, —OCF₃, oxo, 1,2-methylenedioxy, —N(R¹¹)₂, —SR¹¹, —S(O)R¹¹, —S(O)N(R¹¹)₂, —SO₂R¹, —C(O)R¹¹, —CO₂R¹¹ C(O)N(R¹¹)₂, —N(R¹¹)C(O)R′, —N(R¹¹)C(O)OR¹¹, —N(R¹¹)C(O)N(R¹¹)₂, or —OC(O)N(R¹¹)₂,;

R¹¹ is hydrogen, (C₁-C₆)-alkyl, (C₂-C₆)-alkenyl or alkynyl, or (C₃-C₆)cycloalkyl;

P1 and P2 each are independently:

absent; or

aliphatic;

R1 and R2 each are independently:

absent; or

R;

W is five to eleven membered monocyclic or bicyclic, aromatic or non-aromatic ring having zero to three heteroatoms independently selected from O, S, N, or NH, wherein W has up to 3 J substituents.

It is another object of the present invention to provide compositions comprising inhibitors of BACE.

It is also an object of the present invention to provide compounds and compositions useful in treating diseases mediated by BACE.

It is yet another object of the present invention to provide methods for treating Alzheimer's Disease and related neurological diseases.

### BRIEF DESCRIPTION OF THE DRAWINGS

FIG. 1 depicts the interaction between binding sites/subsites of BACE and four features of the inhibitors of the present invention, namely: first hydrogen bonding moiety (“HB-1”), second hydrophobic moiety (“HPB-2”), third hydrophobic moiety (“HPB-3”) and a fourth hydrophobic moiety (“HPB-4”).

FIG. 2 depicts the interaction between binding sites/subsites of BACE and five features of the inhibitors of the present invention, namely: HB-1, first hydrophobic moiety (“HPB-1”), HPB-2, HPB-3 and HPB-4.

FIG. 3 depicts the interaction between binding sites/subsites of BACE and six features of the inhibitors of the present invention, namely: HB-1, HPB-1, HPB-2, HPB-3, HPB-4 and a second hydrogen-bonding moiety (“HB-2”).

FIG. 4 depicts the interaction between binding sites/subsites of BACE and six features of the inhibitors of the present invention, namely: HB-1, HPB-1, HPB-2, HPB-3, HPB-4 and a third hydrogen bonding moiety (“HB-3”).

FIG. 5 depicts the interaction between binding sites/subsites of BACE and seven features of the inhibitors of the present invention, namely: HB-1, HB-2, HB-3, HPB-1, HPB-2, HPB-3 and HPB-4.

### DETAILED DESCRIPTION OF THE INVENTION

Definitions

The following terms are employed herein:

The term “P2 binding pocket” refers to the substrate binding site on the BACE molecule defined by at least Thr-231, Thr-232, Asn-233, Arg-235 and Ser-325.

The term “P2“binding pocket” refers to the substrate binding site on the BACE molecule defined by at least Asn-37, Ala-39, Val-69, Trp-76, Ile-118 and Arg-128.

The term “Flap binding pocket” refers to the pocket defined by at least Trp-76, Phe-108, Phe109, Trp-115 and Ile-102. In the absence of an inhibitor, the flap can be in the closed conformation. However, in the presence of an inhibitor, the flap shifts into a more open conformation to make room for the part of the inhibitor that interacts with the above residues in the flap binding pocket.

The term “hydrophobic” refers to a non-polar moiety that tends not to dissolve in water and is fat-soluble. Hydrophobic moieties include, but are not limited to, hydrocarbons, such as alkanes, alkenes, alkynes, cycloalkanes, ethers, cycloalkenes, cycloalkynes and aromatic compounds, such as aryls, certain saturated and unsaturated heterocycles and moieties that are substantially similar to the side chains of hydrophobic natural and unnatural a-amino acids, including valine, leucine, isoleucine, methionine, phenylanine, a-amino isobutyric acid, alloisoleucine, tyrosine, and tryptophan.

The term “association” refers to a condition of proximity between an inhibitor or portions thereof to the BACE molecule or portions thereof wherein the juxtaposition is energetically favored by electrostatic or van der Waals interactions.

The term “hydrogen bond” refers to a favorable interaction that occurs whenever a suitable donor atom, X, bearing a proton, H, and a suitable acceptor atom, Y, have a separation of ≦3.5 Å and where the angle X—H - - - Y is greater than 90 degrees. Sometimes, a single proton on a donor atom X may form a plurality of suitable acceptor atoms, Y. For example, the proton on a —NH— group may form a separate hydrogen bond with each of the two oxygen atoms in a carboxylate anion. Suitable donor and acceptor atoms are well understood in medicinal chemistry (G. C. Pimentel and A. L. McClellan, The Hydrogen Bond, Freeman, San Francisco, 1960; R. Taylor and O. Kennard, “Hydrogen Bond Geometry in Organic Crystals”, Accounts of Chemical Research, 17, pp. 320-326 (1984)).

The term “hydrogen bonding moiety” refers to a chemical structure containing one or more suitable hydrogen bond donor moieties or hydrogen bond acceptor moieties.

The term “hydrogen bonding donor moiety” refers to a chemical structure containing a suitable hydrogen bond donor atom bearing one or more protons. Examples of donor atoms having one proton are —OH, —SH and —NH—. Examples of donor atoms having more than one proton are —NH₂, [—NH₃]⁺ and [—NH₂—]⁺.

The term “hydrogen bonding acceptor moiety” refers to a chemical structure containing a suitable hydrogen bond acceptor atoms. Examples of acceptor atoms include fluorine, oxygen, sulfur and nitrogen.

The term “stacking interaction” refers to the favorable attractive interactions between two aromatic ring systems, wherein the two rings are juxtaposed such that they are oriented either parallel, perpendicular or at an intermediate angle to each other.

The term “salt bridge” refers to the non-covalent attractive interaction between a positively charged moiety (P) and a negatively charged moiety (N) when the distance between the centers of mass of P and N is between 2 and 6 Angstroms. In calculating the center of mass, atoms which may contain a formal charge and atoms immediately adjacent to these are included. For example, a salt bridge may be formed between the positively charged guanidinium side chain of an arginine residue and the negatively charged carboxylate side chain of a glutamate residue. Salt bridges are well known in medicinal chemistry (L. Stryer, Biochemistry, Freeman, San Francisco, (1975); K. A. Dill, “Dominant Forces in Protein Folding”, Biochemistry, 29, No. 31, pp. 7133-7155, (1990)).

The term “center of mass” refers to a point in three-dimensional space that represents a weighted average position of the masses that make up an object. The distances to or from any given group are calculated from the center of the mass of that group.

The terms “backbone chain” and “backbone” refer to the portion of a polypeptide which comprises the repeating unit —CO—CH—NH—.

The term “minimized geometry” refers to the systematic altering of the atomic geometry of a molecule or molecular complex so that any further minor perturbation of the atomic geometry would cause the total energy of the system as measured by a molecular mechanics force-field to increase. Minimization and molecular mechanics force-fields are well understood in computational chemistry [U. Burkert and N. L. Allinger, Molecular Mechanics, ACS Monograph 177, American Chemical Society, Washington, D.C. 1982 pages 59-78].

The term “strain energy” is used in this application to refer to the difference between the free conformation energy of a compound and the bound conformation energy of that compound when bound to BACE. The strain energy can be determined by the following steps: Determine the bound conformational energy, determine and then subtract from this the un-bound conformational energy. This is the free conformation energy. A more comprehensive definition of strain energy can be found in Bostrom, J., Norrby, P. -O.; Liljefors, T., “Conformational Energy Penalties of Protein-Bound Ligands”, J. Comput. Aided Mol. Design, 1998, 383. The strain energy for binding of a potential inhibitor to BACE is the difference between the free conformation energy and the bound conformation energy. In a preferred embodiment, the strain energy of an inhibitor of the present invention is less than about 10 kcal/mol.

The term “optionally substituted” is used interchangeably with the term “substituted or unsubstituted.”

Unless otherwise indicated, an optionally substituted group may have a substituent at each substitutable atom of the group (including more than one substituent on a single atom), and the identity of each substituent is independent of the others.

The term “aliphatic” or “aliphatic group” as used herein means:

a straight-chain or branched C₁-C₁₂ hydrocarbon chain that is completely saturated or that contains one or more units of unsaturation; or

a monocyclic C₃-C₈ hydrocarbon or bicyclic C₈-C₁₂ hydrocarbon that is completely saturated or that contains one or more units of unsaturation, but which is not aromatic (also referred to herein as “carbocycle”), that has a single point of attachment to the rest of the molecule wherein any individual ring in said bicyclic ring system has three to seven members.

For example, suitable aliphatic groups include, but are not limited to, linear or branched or alkyl, alkenyl, alkynyl groups, carbocyclic groups and hybrids thereof, such as (cycloalkyl)alkyl, (cycloalkenyl)alkyl or (cycloalkyl)alkenyl. In each aliphatic group, up to 2 carbons may be independently replaced by O, S, N, or NH.

The terms “alkyl”, “alkenyl” and “alkynyl” used alone or as part of a larger moiety include both straight and branched chains, wherein up to 2 carbons may be independently replaced by O, S, N, or NH. Unless prefixed with a specific chain length, alkyl, alkenyl and alkynyl contain one to twelve carbon atoms and at least two carbon atoms and one double bond in the case of alkenyl and at least two carbon atoms and one triple bond, in the case of alkynyl.

The terms “halo” and “halogen” used alone or as part of a larger moiety means F, Cl, Br, or I.

The term “heteroatom” includes oxygen and any oxidized form of nitrogen and sulfur, and the quaternized form of any basic nitrogen.

The term “aryl” used alone or as part of a larger moiety as in “aralkyl”, “aralkoxy”, or “aryloxyalkyl”, refers to monocyclic, bicyclic and tricyclic ring systems having a total of five to fourteen ring members, wherein at least one ring in the system is aromatic and wherein each ring in the system contains three to seven ring members. The term “aryl” may be used interchangeably with the term “aryl ring”.

The term “heterocycle”, “heterocyclyl”, or “heterocyclic” as used herein means non-aromatic, monocyclic, bicyclic or tricyclic ring systems having five to fourteen ring members in which one or more ring members is a heteroatom, wherein each ring in the system contains three to seven ring members.

The term “heteroaryl”, used alone or as part of a larger moiety as in “heteroaralkyl” or “heteroarylalkoxy”, refers to monocyclic, bicyclic and tricyclic ring systems having a total of five to fourteen ring members, wherein at least one ring in the system is aromatic, at least one ring in the system contains one or more heteroatoms, and wherein each ring in the system contains three to seven ring members. The term “heteroaryl” may be used interchangeably with the term “heteroaryl ring” or the term “heteroaromatic”.

Further heterocycles and heteraryls are described in A. R. Katritzky and C. W. Rees, eds., Comprehensive Heterocyclic Chemistry: The Structure, Reactions, Synthesis and Use of Heterocyclic Compounds, Vol. 1-8, Pergamon Press, N.Y. (1984).

This invention also envisions the “quaternization” of any basic nitrogen-containing groups of the compounds disclosed herein. The basic nitrogen can be quaternized with any agents known to those of ordinary skill in the art including, for example, lower alkyl halides, such as methyl, ethyl, propyl and butyl chloride, bromides and iodides; dialkyl sulfates including dimethyl, diethyl, dibutyl and diamyl sulfates; long chain halides such as decyl, lauryl, myristyl and stearyl chlorides, bromides and iodides; and aralkyl halides including benzyl and phenethyl bromides. Water or oil-soluble or dispersible products may be obtained by such quaternization.

The BACE inhibitors of this invention may contain one or more “asymmetric” carbon atoms and thus may occur as racemates and racemic mixtures, single enantiomers, diastereomeric mixtures and individual diastereomers. All such isomeric forms of these compounds are expressly included in the present invention. Each stereogenic carbon may be of the R or S configuration. Although specific compounds and scaffolds exemplified in this application may be depicted in a particular stereochemical configuration, compounds and scaffolds having either the opposite stereochemistry at any given chiral center or mixtures thereof are also envisioned.

Combinations of substituents and variables envisioned by this invention are only those that result in the formation of stable compounds.

The term “chemically stable arrangement”, as used herein, refers to a compound structure that possesses stability sufficient to allow manufacture and administration to a mammal by methods known in the art. Typically, such compounds are stable at a temperature of 40° C. or less, in the absence of moisture or other chemically reactive conditions, for at least a week.

The following abbreviations are used herein to represent the features present within the BACE inhibitors of the present invention:

HB-1—a first hydrogen bonding moiety capable of forming up to four hydrogen bonds with the carboxylate oxygen atoms of Asp-228 and Asp-32 of BACE.

HB-2—a second hydrogen-bonding moiety capable of forming a hydrogen bond with the carbonyl oxygen atom of Gly-34 of BACE.

HB-3—a third hydrogen-bonding moiety capable of forming a hydrogen bond with the carbonyl oxygen of Gly-230 of BACE.

HPB-1—a first hydrophobic moiety capable of associating with substantially all residues in the P2 binding pocket of BACE.

HPB-2—a second hydrophobic moiety capable of associating with substantially all residues in the Flap binding pocket of BACE.

HPB-3—a third hydrophobic moiety capable of associating with substantially all residues in the P2′ binding pocket of BACE.

HPB-4—a fourth hydrophobic moiety capable of inducing favorable interactions with the phenyl ring of at least two of Tyr-71, Phe-108 and Trp-76 of BACE.

The present invention provides inhibitors of BACE having the following features:

(a) HB-1;

(b) HPB-4;

and at least one of the following (c) and (d):

(c) HPB-2; and

(d) HPB-3.

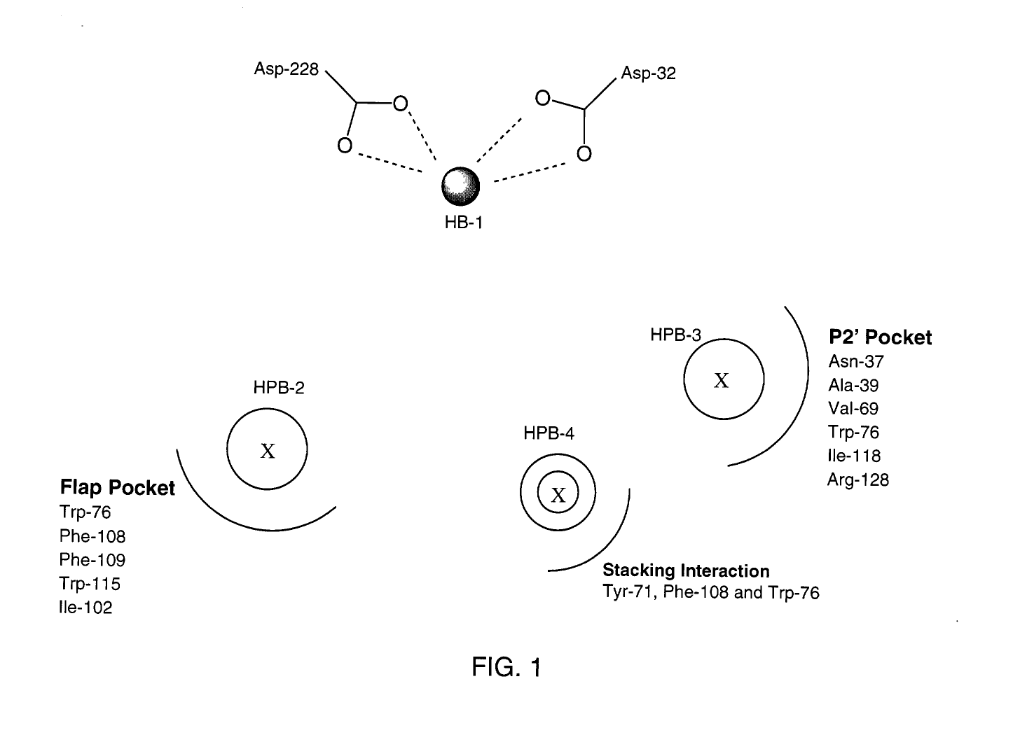

These features and their interaction with the binding sites/subsites of BACE are illustrated in FIG. 1.

According to a preferred embodiment, the inhibitor contains features (a), (b) and (c).

According to another preferred embodiment, the inhibitor contains features (a), (b) and (d).

According to another embodiment, the present invention provides a BACE inhibitor having the following features:

(a) HB-1;

(b) HPB-4;

(c) HPB-2; and

(d) HPB-3.

According to another embodiment, the present invention provides a BACE inhibitor having the following features:

(a) HB-1;

(b) HPB-4;

(c) HPB-1

and at least one of the following (d) and (e):

(d) HPB-2; and

(e) HPB-3.

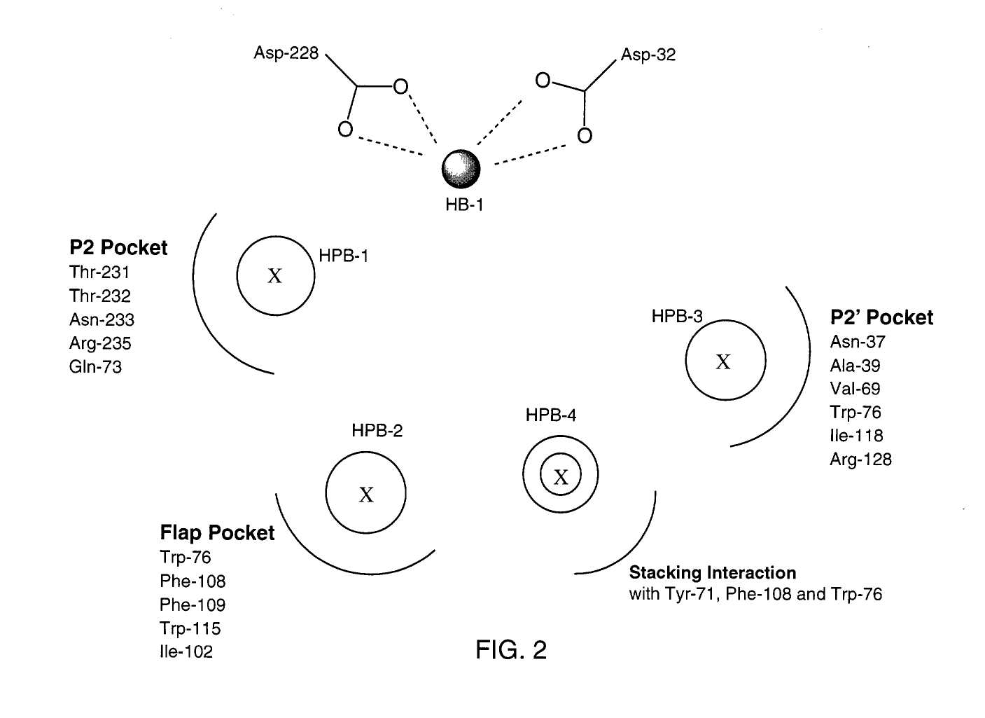

These features and their interaction with the binding sites/subsites of BACE are illustrated in FIG. 2.

According to a preferred embodiment, the inhibitor contains features (a), (b), (c), and (d).

According to another preferred embodiment, the inhibitor contains features (a), (b), (c) and (e).

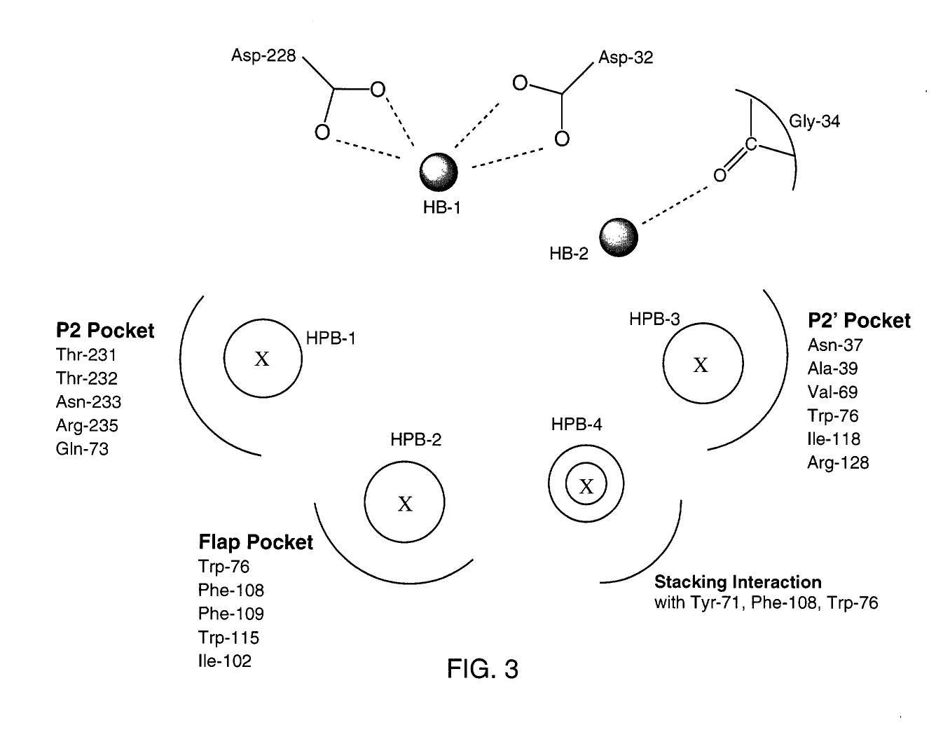

According to a preferred embodiment, the BACE inhibitor of the present invention further comprises a HB-2 feature. This embodiment is illustrated in FIG. 3.

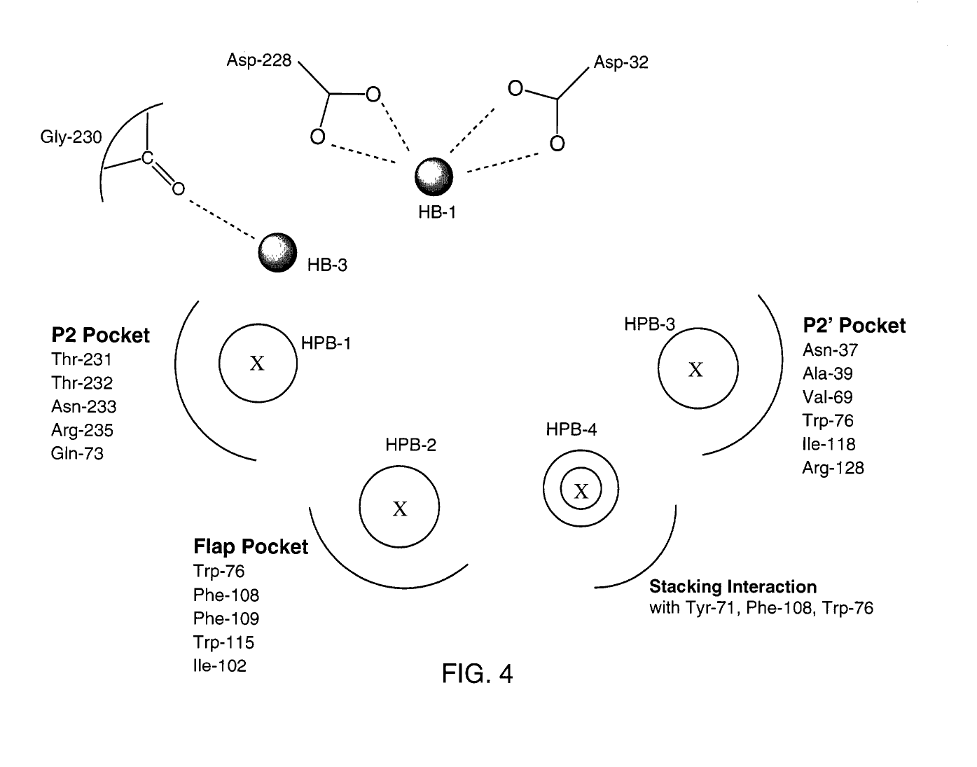

According to another preferred embodiment, the BACE inhibitor of the present invention further comprises a HB-3 feature. This embodiment is illustrated in FIG. 4.

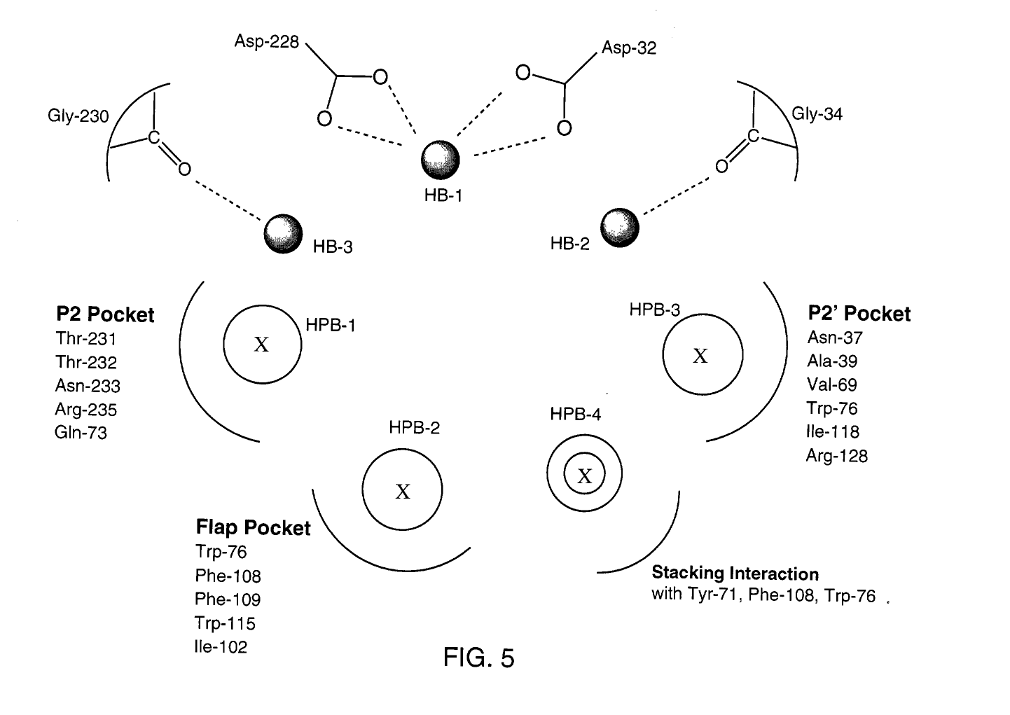

According to another preferred embodiment, the BACE inhibitor of the present invention comprises both, HB-2 and HB-3 features. This embodiment is illustrated in FIG. 5.

Preferably, each of the HB-1, HB-2 and HB-3 is independently less than about 3.5 Å in length.

More preferably, each of HB-1, HB-2 and HB-3 is independently less about 3.0 Å.

According to another embodiment, HB-1 of the BACE inhibitor of the present invention is replaced with a electropositive moiety comprising one or more positively charged atoms, wherein said electropositive moiety forms a salt bridge with the carboxylate oxygen atoms of Asp-228 and Asp-32.

Preferably, the HPB-1 moiety is capable of associating with the P2 binding pocket of BACE such that the distance between the center of mass of the HPB-1 moiety and the C-β atom of substantially all of Thr-231, Thr-232, Asn-233, Arg-235 and Gln-73 is between about 4.0 Å to about 12 Å.

More preferably, the HPB-1 moiety is capable of associating with the P2 binding pocket of BACE such that the distance between the center of mass of the hydrophobic moiety and the C-β atom of substantially all of Thr-231, Thr-232, Asn-233, Arg-235 and Gln-73 is between about 5.0 Å to about 10 Å.

Most preferably, the HPB-1 moiety is capable of associating with the P2 binding pocket of BACE such that the distance between the center of mass of HPB-1 and the C-β atom of substantially all of Thr-231, Thr-232, Asn-233, Arg-235 and Gln-73 is as follows:

Thr-231—between 5.5 to 6.5 Å;

Thr-232—between 6.0 to 6.7 Å;

Asn-233—between 7.0 to 8.5 Å;

Arg-235—between 8.5 to 10.0 Å; and

Gln-73—between 9.0 to 10.0 Å.

Preferably, the HPB-2 moiety is capable of associating with the Flap binding pocket such that the distance between the center of mass of the HPB-2 moiety and the C-β atom of substantially all of Trp-76, Phe-108, Phe-109, Trp-115 and Ile-102 is between about 3.0 Å to about 8.5 Å.

More preferably, the distance between the center of mass of the HPB-2 moiety and the C-β atom of substantially all of Trp-76, Phe-108, Phe-109, Trp-115 and Ile-102 is between about 3.5 Å to about 8.0 Å.

Most preferably, the distance between the center of mass of the HPB-2 moiety and the C-β atom of substantially all of Trp-76, Phe-108, Phe-109, Trp-115 and Ile-102 is as follows:

Trp-76—about 8 Å;

Phe-108—about 3.5 Å;

Phe-109—about 6 Å;

Trp-115—about 8 Å; and

Ile-102—about 6 Å.

Preferably, the HPB-3 moiety binds to the P2′ pocket such that the distance between the center of mass of the HPB-3 moiety and the C-β atom of substantially all of Asn-37, Ala-39, Val-69, Trp-76, Ile-118 and Arg-128 is between 3.5 Å to 8 Å.

More preferably, the distance between the center of mass of the HPB-3 moiety and the C-β atom of substantially all of Asn-37, Ala-39, Val-69, Trp-76, Ile-118 and Arg-128 is between 4 Å to 7.5 Å.

Most preferably, the distance between the center of mass of the HPB-3 moiety and the C-β atom of substantially all of Asn-37, Ala-39, Val-69, Trp-76, Ile-118 and Arg-128 is as follows:

Asn-37—between 4.0 Å to 5.0 Å;

Ala-39—about 6 Å;

Val-69—about 6 Å;

Trp-76—about 7.5 Å;

Ile-118—about 6.7 Å; and

Arg-128—about 6 Å.

Preferably, HPB-4 is an aromatic stacking moiety that interacts favorably with the phenyl ring of at least two of Tyr-71, Phe-108 and Trp-76.

More preferably, the HPB-4 moiety interacts with at least two of Tyr-71, Phe-108 and Trp-76 such that the distance between the center of mass of the HPB-4 moiety and the C-β atom of at least two of Tyr-71, Phe-108 and Trp-76 is between 5.5 Å and 8.5 Å.

More preferably, the HPB-4 moiety interacts with at least two of Tyr-71, Phe-108 and Trp-76 such that the distance between the center of mass of the HPB-4 moiety and the C-β atom of at least two of Tyr-71, Phe-108 and Trp-76 is between 6.0 Å and 8.0 Å.

Most preferably, the HPB-4 moiety interacts with at least two of Tyr-71, Phe-108 and Trp-76 such that the distance between the center of mass of the HPB-4 moiety and the C-β atom of at least two each of Tyr-71, Phe-108 and Trp-76 is as follows:

Tyr-71—about 6.0 Å;

Phe-108—about 5.5 Å; and

Trp-76—about 7 Å.

Preferably, the HPB-4 moiety interacts with Tyr-71 and Phe-108.

More preferably, the HPB-4 moiety interacts with Try-71.

According to a preferred embodiment, within an inhibitor of the present invention, the distance between the HB-1 moiety and other moieties in the inhibitor, when present, is in the range as set forth below in Table 1:

TABLE 1

| | HB-1^{a} |
|---|---|
| HB-2 | 4.0-5.0 |
| HB-3 | 4.0-5.0 |
| HPB-4 | 5.0-6.0 |
| HPB-1 | 7.0-8.5 |
| HPB-2 | 9.0-11.0 |
| HPB-3 | 8.0-11.0 |

^{a}distances in Angstroms (Å)

Preferably, the BACE inhibitor is characterized by a neutral or favorable enthalpic contribution from the sum of all electrostatic interactions between the inhibitor and BACE when the inhibitor is bound thereto.

According to a preferred embodiment, the BACE inhibitor is characterized by an ability to cross the blood-brain barrier. One of skill in the art will be well aware of methods for determining whether an inhibitor has such ability. See, e.g., Murcko et al., “Designing Libraries with CNS activity,” J. Med. Chem., 42(24), pp. 4942-51 (1999).

According to another embodiment, the present invention provides an enzyme-inhibitor complex, wherein said enzyme is BACE and said inhibitor is as described above.

According to another embodiment, the present invention provides a method of inhibiting BACE activity in a mammal, comprising the step of administering to said mammal a BACE inhibitor selected from any one of the above embodiments.

A skilled practitioner will appreciate that there are other aspartic proteinases that share substantially the same inhibitor-enzyme interactions as BACE. Examples of such enzymes include BACE-2, renin, Napsin-A, Napsin-B, Cathepsin-D, Cathepsin-E, Pepsinogen-A and Pepsinogen-C. Thus, when compared to the binding pockets of BACE, each of the above aspartic proteases has a corresponding hydrogen bonding interactions (HB-1, HB-2 and HB-3), a P2 binding pocket, a P2′ binding pocket, a flap-binding pocket and amino acid resides corresponding to Tyr-71, Phe-108 and Trp-76 that have favorable interactions with HPB-4 in BACE. Consequently, one of skill in the art can readily deduce the features of the inhibitors of the present invention are readily applicable to any of the above-mentioned aspartic proteinases based on the analogous binding pockets and interactions.

For example, the amino acid residues in the analogous binding pockets of BACE and Cathepsin-D are recited below in Table 2:

TABLE 2

| Binding | BACE | Inhibitor | Cathepsin-D |
|---|---|---|---|
| Sites | Residues | Features | Residues |
| Hydrogen Bond | Asp-228 | HB-1 | Asp-231 |
| | Asp-32 | | Asp-33 |
| P2 Pocket | | HPB-1 | |
| | Thr-231 | | Thr-234 |
| | Thr-232 | | Ser-235 |
| | Asn-233 | | Leu-236 |
| | Arg-235 | | Val-238 |
| | Gln-73 | | Ser-80 |
| P2′ Pocket | | HPB-3 | |
| | Asn-37 | | Asn-38 |
| | Ala-39 | | Trp-40 |
| | Val-69 | | Ile-76 |
| | Trp-76 | | Leu-83 |
| | Ile-118 | | Ile-134 |
| | Arg-128 | | Val-114 |
| Flap Pocket | | HPB-2 | |
| | Trp-76 | | Leu-83 |
| | Phe-108 | | Phe-126 |
| | Phe-109 | | — |
| | Trp-115 | | Phe-131 |
| | Ile-102 | | Ala-118 |
| Stacking | | HPB-4 | |
| Interaction | | | |
| | Tyr-71 | | Tyr-78 |
| | Phe-108 | | Phe-126 |
| | Trp-76 | | Leu-83 |

Moreover, Trp-78 of BACE and Trp-40 of Cathepsin-D occupy structurally equivalent positions although their main chains are far apart.

Table 2 illustrates the substantial similarity in the enzyme-inhibitor interactions between BACE and Cathepsin-D. The hydrogen bonding residues and the hydrophobic residues present in the BACE binding sites are substantially present in the analogous residues in the corresponding Cathepsin-D binding sites. As a result, the moieties present in the BACE inhibitors of the present invention, and the interactions that they engender, are also present in Cathepsin-D inhibitors. Consequently, one of skill in the art will readily recognize that the binding features that render the inhibitors of the present invention effective against BACE also render them effective against Cathepsin-D. Therefore, the inhibitors of BACE, described above are also useful as inhibitors of other aspartic proteinases in general, and those listed above, in particular.

Thus, according to another embodiment, the present invention provides inhibitors of aspartic proteinases.

According to a more preferred embodiment, the present invention provides inhibitors of BACE-2, Renin, Napsin-A, Napsin-B, Cathepsin-D, Cathepsin-E, Pepsinogen-A and Pepsinogen-C.

According to a preferred embodiment, the present invention provides inhibitors of aspartic proteinases other than renin.

According to yet another embodiment, the present invention provides enzyme-inhibitor complexes, wherein said enzyme is an aspartic proteinase and said inhibitor is as described above. According to a preferred embodiment, said aspartic proteinase in said enzyme-inhibitor complex is BACE-2, BACE, Renin, Napsin-A, Napsin-B, Cathepsin-D, Cathepsin-E, Pepsinogen-A or Pepsinogen-C.

According to another preferred embodiment, said aspartic proteinase in said enzyme-inhibitor complex is other than renin.

According to another embodiment, the present invention provides methods for designing a specific compound as an inhibitor of aspartic proteinases. Such a method is described below for BACE. But, one of skill in the art will readily appreciate that because aspartic proteinases share substantially similar inhibitor-enzyme binding interactions, the methods described below may readily, without undue experimentation, be extended to other aspartic proteinases.

The practitioner skilled in the art will appreciate that there are a number of means to rationally design compound inhibitors of the present invention. These same means may be used to select a candidate compound for screening as a BACE inhibitor. This design or selection may begin with selection of the various moieties that fill the binding pockets described above.

There are a number of ways to select moieties to fill individual binding pockets. These include visual inspection of a physical model or computer model of the active site and manual docking of models of selected moieties into various binding pockets. Modeling software that is well known and available in the art may be used (Guida, W. C. (1994). “Software For Structure-Based Drug Design.” Curr. Opin. Struct. Biology 4: 777-781). These include QUANTA and InsightII [Molecular Simulations, Inc., San Diego, Calif., a division of Pharmacopiea, Inc., Princeton, N.J., 1992], SYBYL [Molecular Modeling Software, Tripos Associates, Inc., St. Louis, Mo., 1992], This modeling step may be followed by energy minimization with standard molecular mechanics force fields such as AMBER [S. J. Weiner, P. A. Kollman, D. A. Case, U. C. Singh, C. Ghio, G. Alagona, and P. Weiner, J. Am. Chem. Soc., vol. 106, pp. 765-784 (1984)], and CHARMM [B. R. Brooks, R. E. Bruccoleri, B. D. Olafson, D. J. States, S Swaminathan, and M. Karplus, J. Comp. Chem. vol. 4, pp. 187-217 (1983)]. In addition, there are a number of more specialized computer programs to assist in the process of selecting the binding moieties of this invention. These include:

1. GRID (Goodford, P. J. A Computational Procedure for Determining Energetically Favorable Binding Sites on Biologically Important Macromolecules. J. Med. Chem., 28, pp. 849-857 (1985)). GRID is available from Oxford University, Oxford, UK.

2. MCSS (Miranker, A.; Karplus, M. Functionality Maps of Binding Sites: A Multiple Copy Simultaneous Search Method. Proteins: Structure, Function and Genetics, 11, pp. 29-34 (1991)). MCSS is available from Molecular Simulations, Inc., San Diego, Calif., a division of Pharmacopiea, Princeton, N.J.

3. AUTODOCK (Goodsell, D. S.; Olsen, A. J. Automated Docking of Substrates to Proteins by Simulated Annealing. PROTEINS: Structure, Function and Genetics, 8, pp. 195-202 (1990)). AUTODOCK is available from the Scripps Research Institute, La Jolla, Calif.

4. DOCK (Kuntz, I. D.; Blaney, J. M.; Oatley, S. J.; Langridge, R.; Ferrin, T. E. A Geometric Approach to Macromolecule-Ligand Interactions. J. Mol. Biol., 161, pp. 269-288 (1982)). DOCK is available from the University of California, San Francisco, Calif.

Once suitable binding moieties have been selected, they can be assembled into a single inhibitor. This assembly may be accomplished by connecting the various moieties to a central scaffold through suitable linkers. The assembly process may, for example, be done by visual inspection followed by manual model building, again using software such as QUANTA or SYBYL. A number of other programs may also be used to help select ways to connect the various moieties. These include:

1. CAVEAT (Bartlett, P. A.; Shea, G. T.; Telfer, S. J.; Waterman, S. CAVEAT: A Program to Facilitate the Structure-Derived Design of Biologically Active Molecules. In “Molecular Recognition in Chemical and Biological Problems,” Special Pub., Royal Chem. Soc., 78, pp. 182-196 (1989)). CAVEAT is available from the University of California, Berkeley, Calif.

2. 3D Database systems such as MACCS-3D (MDL Information Systems, San Leandro, Calif.). This area has been recently reviewed by Martin (Martin, Y. C. 3D Database Searching in Drug Design. J. Med. Chem., 35, pp. 2145-2154 (1992)).

3. HOOK (available from Molecular Simulations, Inc., San Diego, Calif., a division of Pharmacopiea, Princeton, N.J.).

4. Pearlman, D. A. and M. A. Murcko, “Concerts—Dynamic Connection of Fragments as an Approach to De-Novo Ligand Design.” Journal of Medicinal Chemistry 39: pp. 1651-1663 (1993).

In addition to the above computer assisted modeling of inhibitor compounds, the inhibitors of this invention may be constructed “de novo” using either an empty active site or optionally including some portions of a known inhibitor (Walters, W. P., M. T. Stahl, et al. (1998). “Virtual Screening—An Overview.” Drug Disovery Today 3: 160-178). Such methods are well known in the art. They include, for example:

1. LUDI (Bohm, H. J. The Computer Program LUDI: A New Method for the De Novo Design of Enzyme Inhibitors. J. Comp. Aid. Molec. Design., 6, 61-78 (1992)). LUDI is available from Biosym Technologies, Princeton, N.J.

2. LEGEND (Nishibata, Y., Itai, A., Tetrahedron, 47, 8985 (1991)). LEGEND is available from Molecular Simulations, Princeton, N.J.

3. LeapFrog (available from Tripos associates, St. Louis, Mo.).

4. Clark, D. E., A. D. Frenkel, et al. (1995). “PRO_LIGAND: An Approach to De Novo Drug Design. 1. Application to the Design of Organic Molecules.” J. Comput. Aided Mol. Design 9, 13-32.

5. Miller, M. D., S. K. Kearsley, et al. (1994). “FLOG—A system to select quasi-flexible ligands complementary to a receptor of known three-dimensional structure.” Journal of Computer-Aided Molecular Design 8, pp. 153-174.

A number of techniques commonly used for modeling drugs may be employed (For a review, see: Charifson, P. S., editor, Practical Application of Computer-Aided Drug Design, Marcel Dekker, Inc., 1997; Bohacek R S, McMartin C, Guida W C., “The art and practice of structure-based drug design: a molecular modeling perspective”, Med. Res. Rev., 16, pp. 3-50 (1996); and Cohen, N. C.; Blaney, J. M.; Humblet, C.; Gund, P.; Barry, D.C., “Molecular Modeling Software and Methods for Medicinal Chemistry”, J. Med. Chem., 33, pp. 883-894 (1990)). There are likewise a number of examples in the chemical literature of techniques that can be applied to specific drug design projects. For a review, see: Navia, M. A. and Murcko, M. A., “The Use of Structural Information in Drug Design”, Current Opinions in Structural Biology, 2, pp. 202-210 (1992). Some examples of these specific applications include: Tung, R. D. et al., “Design and Synthesis of Amprenavir, A Novel HIV Protease Inhibitor”, in Protease Inhibitors in AIDS Therapy, ed. Ogden, R. C. and Flexner, C. W., Mercel Dekker, Inc., N.Y. Chapt. 6, pp. 101-118 (2000); Baldwin, J. J. et al., “Thienothiopyran-2-sulfonamides: Novel Topically Active Carbonic Anhydrase Inhibitors for the Treatment of Glaucoma”, J. Med. Chem., 32, pp. 2510-2513 (1989); Appelt, K. et al., “Design of Enzyme Inhibitors Using Iterative Protein Crystallographic Analysis”, J. Med. Chem., 34, pp. 1925-1934 (1991); and Ealick, S. E. et al., “Application of Crystallographic and Modeling Methods in the Design of Purine Nucleotide Phosphorylase Inhibitors” Proc. Nat. Acad. Sci. USA, 88, pp. 11540-11544 (1991).

Using the novel combination of steps of the present invention, the skilled artisan can advantageously avoid time consuming and expensive experimentation to determine enzymatic inhibition activity of particular compounds. The method also is useful to facilitate rational design of BACE inhibitors and therapeutic and prophylactic agents against BACE mediated diseases. Accordingly, the present invention envisions such inhibitors and uses.

A variety of conventional techniques may be used to carry out each of the above evaluations as well as the evaluations necessary in screening a candidate compound for BACE inhibiting activity. Generally, these techniques involve determining the location and binding proximity of a given moiety, the occupied space of a bound inhibitor, the deformation energy of binding of a given compound and electrostatic interaction energies. Examples of conventional techniques useful in the above evaluations include: quantum mechanics, molecular mechanics, molecular dynamics, Monte Carlo sampling, systematic searches and distance geometry methods (G. R. Marshall, Ann. Rev. Pharmacol. Toxicol., 27, p. 193 (1987)). Specific computer software has been developed for use in carrying out these methods. Examples of programs designed for such uses include: Gaussian 92, revision E.2 (M. J. Frisch, Gaussian, Inc., Pittsburgh, Pa. ©1993); AMBER, version 4.0 (P.A. Kollman, University of California at San Francisco, ©1993); QUANTA/CHARMM and Insight II/Discover [Molecular Simulations, Inc., San Diego, Calif., a division of Pharmacopiea, Inc., Princeton, N.J. ©1992]. These programs may be implemented, for instance, using a Silicon Graphics Octane workstation or IBM RISC/6000 workstation model 550. Other hardware systems and software packages will be known and of evident applicability to those skilled in the art.

Different classes of BACE inhibitors of this invention may also use different scaffolds or core structures, but all of these cores will allow the necessary moieties to be placed in the active site such that the specific interactions necessary for binding may be obtained. These compounds are best defined in terms of their ability to match the pharmacophore, i.e., their structural identity relative to the shape and properties of the active site of BACE. Distances between the different moieties of the pharmacophore may be readily determined using any modeling software and other suitable chemical structure software. In addition, specialized, commercially available pharmacophore modeling software enables one to determine pharmacophore models from a variety of structural information and data. This software may also be used to search a database of three-dimensional structures in order to identify compounds that meet the above specific pharmacophore requirements. Examples of this software include:

1. DISCO (Martin, Y. C., Bures, M. G., Danaher, E. A., DeLazzer, J., Lico, A., Pavlik, P. A., J. Comput. Aided Mol. Design, 1993, 7, 83). DISCO is available from Tripos Associates, St. Louis, Mo.

2. CHEM-X which is developed and distributed by Chemical Design Ltd, Oxon, UK and Mahwah, N.J.

3. APEX-3D which is part of the Insight molecular modeling program, distributed by Molecular Simulations, Inc., San Diego, Calif.

4. CATALYST (Sprague, P. W., Perspectives in Drug Discovery and Design, 1995, 3, 1; Muller, K., Ed., ESCOM, Leiden) CATALYST is distributed by Molecular Simulations, Inc., San Diego, Calif.

5. UNITY, which is available from Tripos Associates, St. Louis, Mo.

A method known in the art utilizes scaffolds from known drugs in the market. These “drug-like” scaffolds may provide the requisite cores useful in tailoring the requisite moieties to match the pharmacophore such that their interactions with the active site of BACE is optimal. See, e.g., WO 98/57155, and Fesjo, J., et al., “The SHAPES Strategy: an NMR-based approach for lead generation in drug discovery,” Chemistry & Biology, 6: 755-769 (1999).

According to a preferred embodiment, the BACE inhibitor of the present invention has the following formula (I):

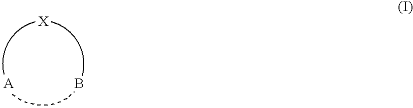

wherein:

X is ═N—, —N(R)—, —NH—, —NH₂ or —CHOH;

wherein R is H, (C₁-C₆) alkyl, (C₂-C₆) alkenyl or alkynyl;

A and B, taken together with X, form a cycloalkyl or aromatic or non-aromatic heterocyclic ring; or

A and B, taken together with X, form an acyclic chain containing up to 10 atoms in the chain;

wherein the A—X—B moiety is optionally fused with a non-aromatic or aromatic carbocyclic or heterocyclic ring; and

wherein the A—X—B moiety contains up to 3 substituents having the formula —(L)ₙ—M, wherein:

n is 0 or 1;

L is a suitable linker, optionally containing a hydrogen bonding moiety; and

M is independently selected from HB-1, HB-2, HPB-1, HPB-2, HPB-3 or HPB-4.

According to a preferred embodiment, M is an aromatic stacking moiety such as a carbocyclic aromatic or heterocyclic aromatic moiety.

According to a preferred embodiment, suitable linker R, when present, has the formula:

—(T¹)ₘ—L¹—(T²)ₘ—

wherein:

m is 0 or 1;

T¹ and T² are independently selected from C₁-C₆ alkyl, C₂-C₆ alkenyl or alkynyl, wherein any carbon in T¹ and T² may be replaced by a heteroatom group in a chemically stable arrangement selected from —O—, —S—, —NH—, —NR′—, —C(O)—, —S(O)— and —S(O)₂—;

R′ is H or aliphatic; and

L¹ is —CH(OH)—, —CH(OR)—, —CH(NRR)—, —CO—, —O—, —NR′—, —SO—, —SO₂—, —NR′SO₂—, —CONR′—, —NR′—CO—, —O—CO—, —CO—O—, —O—CO—NR′—, —NR′—CO—O—, or —NR′—CO—NR′—.

More preferably, suitable linker R is —CH₂—, —O—, —S—, —SO—, —SO₂—, —NR′—, —C(O)O—, —OC(O)—, —C(O)NR′—, —NR′—C(O)—, —O—C(O)—O—, —O—C(O)—NR′—, —NR′—C(O)—NR′—, —NR′—C(O)—O—, —SO—NR′, —NR′—SO—, —NR′—SO₂—, —SO₂—NR′—, —CHOR′—, —CHNR′—, or —C(O)—.

Preferred embodiments of formula (I) include the following:

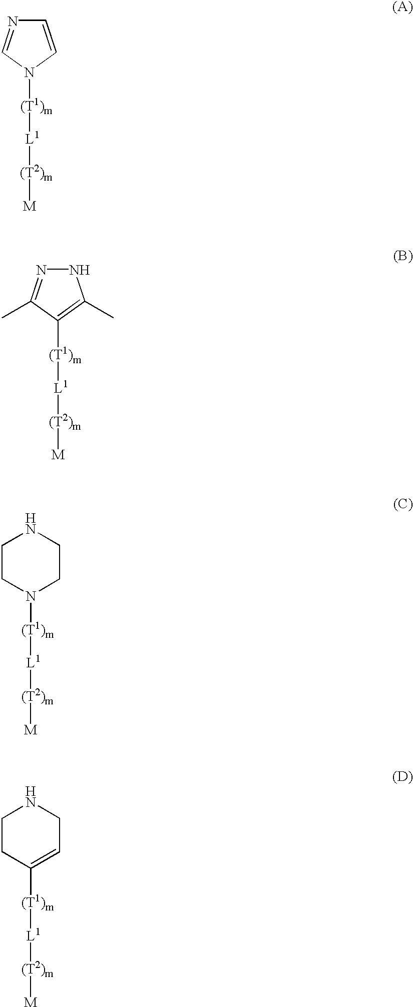

wherein T¹, T², R, L¹ and M are as defined above;

M is an aromatic carbocyclic or aromatic heterocyclic moiety; and

the ring attached to T1 is optionally substituted with up to 2 substituents.

In each of formula (A) and formula (B), preferably:

T¹ is C₁-C₆ alkyl (i.e., m is 1);

L¹ is O, NH or S;

T2 is absent (i.e., m is zero); and

M is a phenyl ring optionally substituted with up to 4 substituents selected from (C₁-C₆) alkyl, (C₂-C₆) alkenyl, —OMe or halogen.

In formula (C), preferably:

T¹ is (C₁-C₆) alkyl (i.e., m is 1); more preferably T¹ is methyl;

R is (C₁-C₆) alkyl;

L¹ is CHOH;

T² is (C₁-C₆) alkyl (i.e., m is 1); more preferably T2 is methyl; and

M is a phenyl ring optionally substituted with up to 4 substituents selected from (C₁-C₆) alkyl, (C₂-C₆) alkenyl, —OMe or halogen.

According to one embodiment of the present invention, preferred compounds of formula (A), formula (B) or formula (C) include the following:

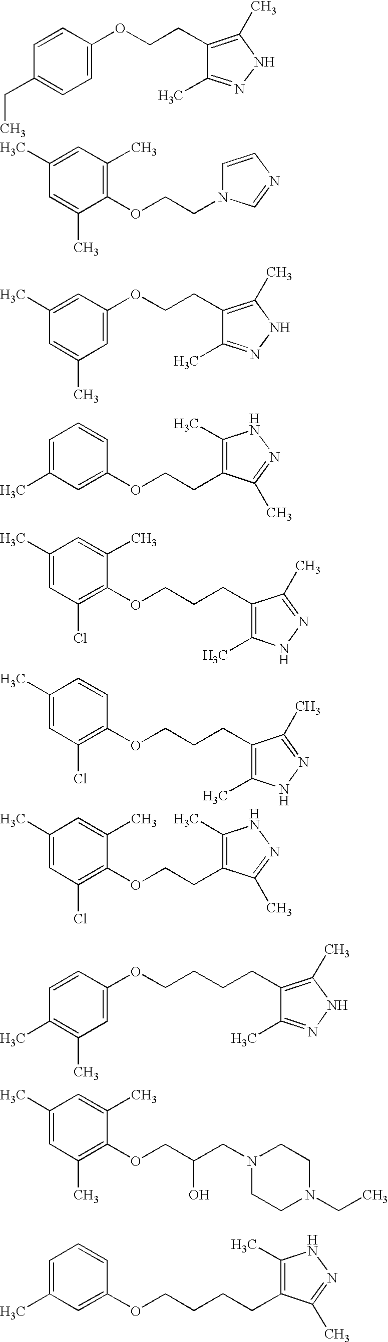

According to another embodiment, the present invention provides a method of inhibiting BACE activity in a mammal, comprising the step of administering to said mammal a compound of formula IA:

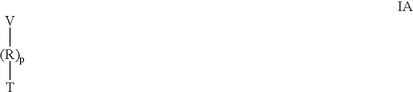

or a pharmaceutically acceptable salt thereof, wherein:

V is a 3-4 membered acyclic group or a 5-7 membered, fully or partially saturated cyclic group;

wherein V comprises a first moiety selected from NH, CH—OH, or a CH—NH₂, and a second moiety selected from carbon, CH, or N;

wherein said first moiety and said second moiety in V are non-adjacent; and

V is attached to R through said second moiety;

wherein V is optionally substituted with R¹⁰;

R is a suitable linker;

p is 0 or 1;

R¹⁰ is P1-R1-P2-R2-W;

T is a five to eleven membered monocyclic or bicyclic, aromatic or non-aromatic ring having zero to three heteroatoms independently selected from O, S, N or NH, wherein T has at least one R¹⁰ substituent and up to three more substituents selected from R¹⁰ or J;

J is halogen, —R′, —OR′, —NO₂, —CN, —CF₃, —OCF₃, oxo, 1,2-methylenedioxy, —N(R′)₂, —SR′, —S(O)R′, —S(O)N(R′)₂, —SO₂R′, —C(O)R′, —CO₂R′, —C(O)N(R′)₂, —N(R′)C(O)R′, —N(R′)C(O)OR′, —N(R′)C(O)N(R′)₂, or —OC(O)N(R′)₂, wherein R′ is independently selected from hydrogen, aliphatic, heterocyclyl, heterocycly-alkyl, aryl, aralkyl, heteroaryl, or heteroaralkyl;

wherein R′ is optionally substituted with up to 3 substituents selected independently from —R¹¹, —OR¹¹, —NO₂, —CN, —CF₃, —OCF₃, oxo, 1,2-methylenedioxy, —N(R¹¹)₂, —SR¹¹, —S(O)R¹¹, —S(O)N(R¹¹)₂, —SO₂R¹¹, —C(O)R”, —CO₂R , C(O)N(R¹¹)₂, —N(R¹¹)C(O)R′, —N(R¹¹)C(O)OR¹¹, —N(R¹¹)C(O)N(R¹¹)₂, or —OC(O)N(R¹¹)₂,;

R¹¹ is hydrogen, (C₁-C₆)-alkyl, (C₂-C₆)-alkenyl or alkynyl, or (C₃-C₆)cycloalkyl;

P1 and P2 each are independently:

absent; or

aliphatic;

R1 and R2 each are independently:

absent; or

R;

W is five to eleven membered monocyclic or bicyclic, aromatic or non-aromatic ring having zero to three heteroatoms independently selected from O, S, N, or NH, wherein W has up to 3 J substituents.

According to one embodiment of the present invention, p is 0. According to another embodiment of the present invention, p is 1.

According to one embodiment, suitable linker R, when present, has the formula:

—(T¹)ₘ—L¹—(T²)ₘ—,

wherein:

m is 0 or 1;

T¹ and T² are independently selected from C₁₋₆ alkyl, C₂₋₆ alkenyl or alkynyl, wherein any carbon in T¹ and T² may be replaced by a heteroatom group in a chemically stable arrangement selected from —O—, —S—, —NH—, —NR′—, —C(O)—, —S(O)— and —S(O)₂—;

R′ is independently selected from hydrogen, aliphatic, cycloalkyl, cycloalkyl-alkyl, heterocyclyl, aryl, aralkyl, heteroaryl, or heteroaralkyl;

wherein R′ is optionally substituted with up to 3 substituents selected independently from —R¹¹, —OR¹¹, —NO₂, —CN, —CF₃, —OCF₃, oxo, 1,2-methylenedioxy, —N(R¹¹)₂, —SR¹¹, —S(O)R¹¹, —S(O)N(R¹¹)₂, —SO₂R¹¹, —C(O)R¹¹, —CO₂R¹¹, —C(O)N(R¹¹)₂, —N(R¹¹)C(O)R′, —N(R¹¹)C(O)OR′, —N(R¹¹)C(O)N(R¹¹)₂, or —OC(O)N(R¹)₂,;

R¹¹ is hydrogen, (C₁-C₆)-alkyl, (C₂-C₆)-alkenyl or alkynyl, or (C₃-C₆)cycloalkyl; and

L¹ is selected from —CH(OR′)—, —CH(NR′R′)—, —C(O)—, —O—, —NR′—, —SO—, —SO₂—, —NR′SO₂—, —CONR′—, —NR′—C(O)—, —O—C(O)—, —C(O)—O—, —O—C(O)—NR′—, —NR′—C(O)—O—, and —NR′C(O)NR′—.

More preferably, R is —CH₂—, —O—, —S—, —SO—, —SO₂—, —NR′—, —C(O)O—, —OC(O)—, —C(O)NR′—, —NR′C(O)—, —O—, —OC(O)NR′—, —NR′C.(O)NR′—, —NR′C.(O)O—, —SO—NR′, —NR′SO—, —NR′SO₂—, —SO₂NR′—, —CHOR′—, —CHNR′—, or —C(O)—.

According to a preferred embodiment of compounds of formula (IA):

R¹⁰ is P1-R1-P2-R2-W,

wherein one of P1 and P2 is absent and the other of P1 and P2 is aliphatic, and/or one of R1 and R2 is absent and the other of R1 and R2 is R.

According to one embodiment, W is a five to seven membered monocyclic, aromatic or non-aromatic ring having zero to three heteroatoms independently selected from O, S, N, or NH, wherein W has up to 3 substituents independently selected from J.

According to a preferred embodiment, W is a five to six membered monocyclic, aromatic ring having one to three heteroatoms independently selected from O, S, N, or NH, wherein W has up to 3 substituents independently selected from J. Preferred five or six membered aromatic rings having one to three heteroatoms include 2-furanyl, 3-furanyl, 3-furazanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 1-pyrrolyl, 2-pyrrolyl, 3-pyrrolyl, 1-pyrazolyl, 2-pyrazolyl, 3-pyrazolyl, 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, or 3-thienyl.

According to another preferred embodiment, W is a five to six membered monocyclic, non-aromatic ring having one to three heteroatoms independently selected from O, S, N, or NH, wherein W has up to 3 substituents independently selected from J. Preferred five or six membered non-aromatic rings having one to three heteroatoms include 2-tetrahydrofuranyl, 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, or N-substituted diazolonyl.

According to another preferred embodiment, W is a five to seven membered monocyclic, aromatic or non-aromatic ring having zero heteroatoms independently selected from O, S, N, or NH, wherein W has up to 3 substituents independently selected from J. More preferably, W is cyclopentyl, cyclohexyl, or phenyl, wherein W has up to 3 substituents independently selected from J. Most preferably, W is phenyl, wherein W has up to 3 substituents independently selected from J.

According to one embodiment, W is an eight to eleven membered bicyclic ring, wherein either or both rings may be aromatic or non-aromatic, and either or both rings may have zero to three heteroatoms independently selected from O, S, N, or NH, wherein W has up to 3 substituents independently selected from J. Preferred aromatic or non-aromatic bicyclic rings having one to three heteroatoms include naphthyl, decalinyl, tetrahydro-naphthyl, 3-1H-benzimidazol-2-one, (1-substituted)-2-oxo-benzimidazol-3-yl, 1-phthalimidinyl, benzoxanyl, benzopyrrolidinyl, benzopiperidinyl, benzoxolanyl, benzothiolanyl, benzothianyl, indolinyl, chromanyl, phenanthridinyl, tetrahydroquinolinyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, indolyl, isoindolyl, acridinyl, benzoisoxazolyl, tetrahydroquinolinyl, tetrahydroisoquinolinyl, or pyrido[3,4-d]pyrimidinyl.

According to a yet more preferred embodiment, R¹⁰ is independently selected from substituents present in compounds in any of Table 1 through Table 5, infra.

According to one embodiment, V in compounds of formula IA is a 3-4 membered acyclic group, wherein V comprises a first moiety selected from NH, CH—OH, or a CH—NH₂, and a second moiety selected from carbon, CH, or N;

wherein said first moiety and said second moiety in V are non-adjacent; and

V is attached to R through said second moiety; wherein V is optionally substituted with R^{10.}

According to another embodiment, V in compounds of formula IA is 5-7 membered cyclic group, wherein V comprises a first moiety selected from NH, CH—OH, or a CH—NH₂, and a second moiety selected from carbon, CH, or N;

wherein said first moiety and said second moiety in V are non-adjacent; and

V is attached to R through said second moiety; wherein V is optionally substituted with R^{10.}

According to yet another embodiment, V in compounds of formula IA is a 5 membered cyclic group, wherein V comprises a first moiety selected from NH, CH—OH, or a CH—NH₂, and a second moiety selected from carbon, CH, or N;

wherein said first moiety and said second moiety in V are non-adjacent; and

V is attached to R through said second moiety; wherein V is optionally substituted with R¹⁰.

According to a preferred embodiment, V in compounds of formula IA is selected from IA-1 through IA-9 shown below:

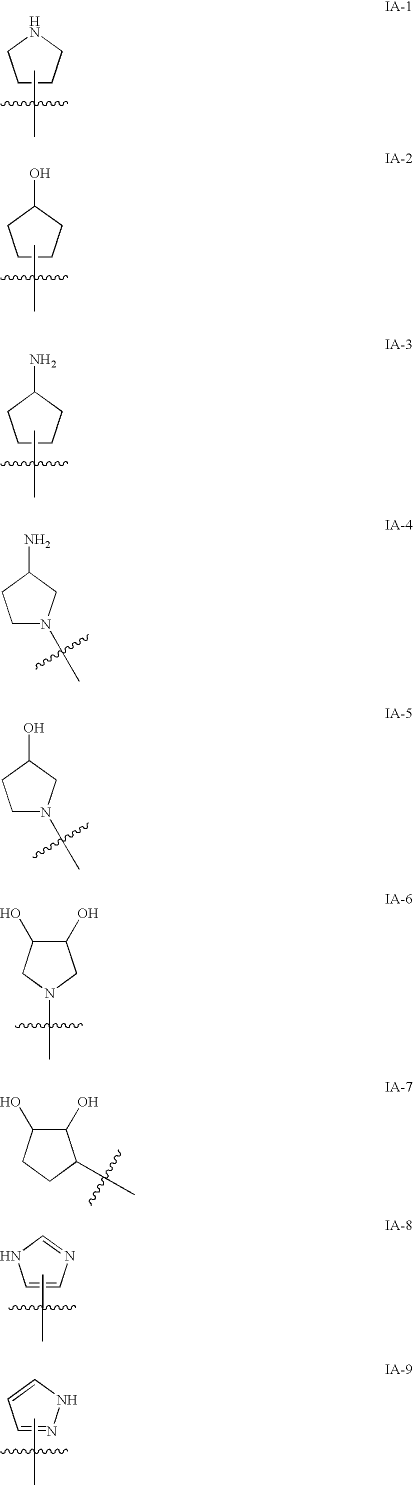

Representative compounds of formula IA are listed below in Table 1.

TABLE 1
Compounds of Formula IA

| | |
|---|---|
| 226 | N4-Methyl-N4-(2-methylamino-ethyl)- |
| | N3-naphthalen-2-ylmethyl-4′- |
| | trifluoromethyl-biphenyl-3,4-diamine |

According to yet another embodiment, V in compounds of formula IA is a 6-7 membered cyclic group, wherein V comprises a first moiety selected from NH, CH—OH, or a CH—NH₂, and a second moiety selected from carbon, CH, or N;

wherein said first moiety and said second moiety in V are non-adjacent; and

V is attached to R through said second moiety; wherein V is optionally substituted with R^{10.}

According to another preferred embodiment, V in compounds of formula IA is selected from formula IB-1 to formula IB-6 shown below:

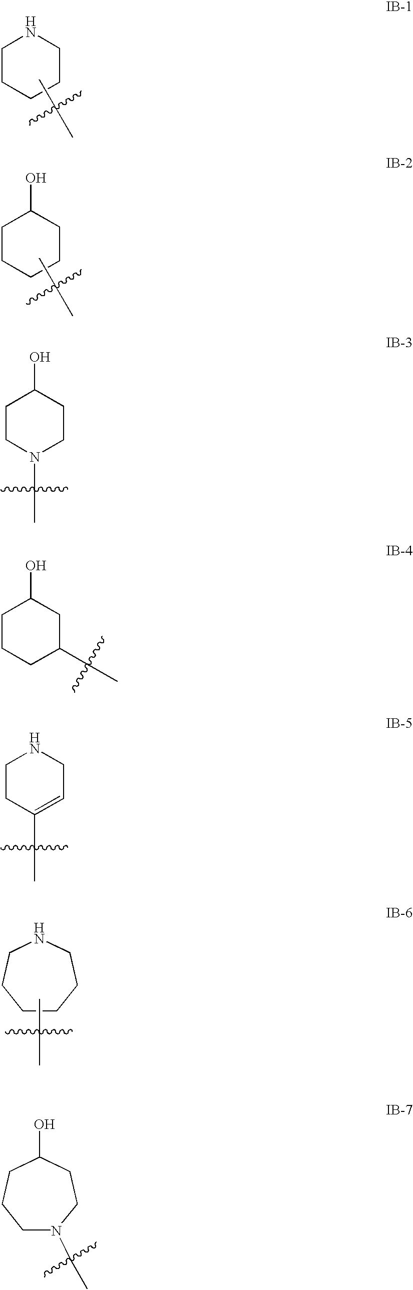

More preferably, V in compounds of formula IA is selected from IB-1 or IB-5. Most preferably, V is IB-5.

Representative compounds of formula IB are listed below in Table 2.

TABLE 2
Compounds of Formula IB

| | |
|---|---|
| 203 | 4-[4-(2-Trifluoromethyl-phenoxymethyl)-phenyl]- |
| | piperidine-3-carboxylic acid (furan-2- |
| | ylmethyl)-amide |
| 205 | (3,4-Dihydro-1H-isoquinolin-2-yl)-{4-[4-(2- |
| | trifluoromethyl-phenoxymethyl)-phenyl]- |
| | piperidin-3-yl}-methanone |
| 207 | 2-({4-[4-(2-Trifluoromethyl-phenoxymethyl)- |
| | phenyl]-piperidin-3-ylmethyl}-carbamoyl)- |
| | cyclohexanecarboxylic acid |
| 208 | 4-[4-(2-Trifluoromethyl-phenoxymethyl)-phenyl]- |
| | piperidine-3-carboxylic acid 2- |
| | trifluoromethoxy-benzylamide |
| 209 | 4-[4-(2-Trifluoromethyl-phenoxymethyl)-phenyl]- |
| | piperidine-3-carboxylic acid (1,2,3,4- |
| | tetrahydro-naphthalen-1-yl)-amide |
| 210 | 2,4-Bis-benzyloxy-5-(1,2,3,6-tetrahydro- |
| | pyridin-4-yl)-pyrimidine |
| 211 | 4-[4-(2-Trifluoromethyl-phenoxymethyl)-phenyl]- |
| | piperidine-3-carboxylic acid benzhydryl-amide |
| 212 | 2-{4-[4-(2-Trifluoromethyl-phenoxymethyl)- |
| | phenyl]-piperidin-3-ylmethyl}-isoindole-1,3- |
| | dione |
| 213 | 3-({4-[4-(2-Trifluoromethyl-phenoxymethyl)- |
| | phenyl]-piperidin-3-ylmethyl}-carbamoyl)- |
| | naphthalene-2-carboxylic acid |
| 214 | 6-Phenyl-2-piperidin-4-yl-3-(2-trifluoromethyl- |
| | benzyl)-3H-pyrimidin-4-one |
| 215 | 4-[4-(2-Trifluoromethyl-phenoxymethyl)-phenyl]- |
| | piperidine-3-carboxylic acid (naphthalen-1- |
| | ylmethyl)-amide |
| 216 | 4-[4-(2-Trifluoromethyl-phenoxymethyl)-phenyl]- |
| | piperidine-3-carboxylic acid naphthalen-2- |
| | ylamide |
| 218 | 3-Naphthalen-2-ylmethyl-6-phenyl-2-piperidin-4- |
| | yl-3H-pyrimidin-4-one |
| 219 | 4-[4-(2-Trifluoromethyl-phenoxymethyl)-phenyl]- |
| | piperidine-3-carboxylic acid (1,2,3,4- |
| | tetrahydro-naphthalen-2-yl)-amide |
| 220 | 4-[4-(2-Trifluoromethyl-phenoxymethyl)-phenyl]- |
| | piperidine-3-carboxylic acid benzyl-naphthalen- |
| | 2-yl-amide |
| 221 | Naphthalene-1-carboxylic acid {4-[4-(2- |
| | trifluoromethyl-phenoxymethyl)-phenyl]- |
| | piperidin-3-ylmethyl}-amide |
| 222 | Naphthalene-2-carboxylic acid {4-[4-(2- |
| | trifluoromethyl-phenoxymethyl)-phenyl]- |
| | piperidin-3-ylmethyl}-amide |
| 223 | {1-Benzyl-2-oxo-2-[2-({4-[4-(2-trifluoromethyl- |
| | phenoxymethyl)-phenyl]-piperidin-3-ylmethyl}- |
| | carbamoyl)-pyrrolidin-1-yl]-ethyl}-carbamic |
| | acid benzyl ester |
| 224 | 1-Naphthalen-1-yl-3-{4-[4-(2-trifluoromethyl- |
| | phenoxymethyl)-phenyl]-piperidin-3-ylmethyl}- |
| | urea |
| 225 | (2-Phenyl-1-{[({4-[4-(2-trifluoromethyl- |
| | phenoxymethyl)-phenyl]-piperidin-3-ylmethyl}- |
| | carbamoyl)-methyl]-carbamoyl}-ethyl)-carbamic |
| | acid benzyl ester |
| 228 | {4-[4-(2-Trifluoromethyl-phenoxymethyl)- |
| | phenyl]-piperidin-3-ylmethyl}-carbamic acid |
| | naphthalen-2-yl ester |
| 229 | {4-[4-(2-Trifluoromethyl-phenoxymethyl)- |
| | phenyl]-piperidin-3-ylmethyl}-carbamic acid |
| | naphthalen-1-yl ester |
| 230 | {1-(1H-Indol-3-ylmethyl)-2-oxo-2-[2-{{4-[4-(2- |
| | trifluoromethyl-phenoxymethyl)-phenyl]- |
| | piperidin-3-ylmethyl}-carbamoyl)-pyrrolidin-1- |
| | yl]-ethyl}-carbamic acid 9H-fluoren-9-ylmethyl |
| | ester |
| 231 | Naphthalene-2-sulfonic acid {4-[4-(2- |
| | trifluoromethyl-phenoxymethyl)-phenyl]- |
| | piperidin-3-ylmethyl}-amide |
| 232 | 1-Naphthalen-2-yl-3-{4-[4-(2-trifluoromethyl- |
| | phenoxymethyl)-phenyl]-piperidin-3-ylmethyl}- |
| | urea |
| 301 | 4-[4-Naphthalen-1-yl-2,5-bis-(2- |
| | trifluoromethyl-phenoxymethyl)-phenyl]-1,2,3,6- |
| | tetrahydro-pyridine |
| 302 | 4-Biphenyl-4-yl-3-(naphthalen-2-yloxymethyl)- |
| | 1,2,3,6-tetrahydro-pyridine |
| 303 | 4-[2,5-Bis-(2-trifluoromethyl-phenoxymethyl)- |
| | phenyl]-1,2,3,6-tetrahydro-pyridine |
| 304 | 4-[2,6-Bis-(2-trifluoromethyl-phenoxymethyl)- |
| | phenyl]-1,2,3,6-tetrahydro-pyridine |
| 305 | 6-Benzyloxy-9-naphthalen-2-ylmethyl-2,3,4,9- |
| | tetrahydro-1H-b-carboline |
| 306 | 4-[2,5-Bis-(2-trifluoromethyl-phenoxymethyl)- |
| | biphenyl-4-yl]-1,2,3,6-tetrahydro-pyridine |
| 307 | 4-[2,5-Bis-(naphthalen-2-yloxymethyl)-biphenyl- |
| | 4-yl]-1,2,3,6-tetrahydro-pyridine |
| 308 | N-Naphthalen-2-yl-2-{1,2,3,6-tetrahydro- |
| | pyridin-4-yl)-4-(2-trifluoromethyl- |
| | phenoxymethyl)-benzamide |
| 309 | N-(4-Methoxy-naphthalen-2-yl)-2-(1,2,3,6- |
| | tetrahydro-pyridin-4-yl)-4-(2-trifluoromethyl- |
| | phenoxyraethyl)-benzamide |
| 310 | N-(5-Amino-naphthalen-1-yl)-2-(1,2,3,6- |
| | tetrahydro-pyridin-4-yl)-4-(2-trifluoromethyl- |
| | phenoxymethyl)-benzamide |
| 311 | N-(3-Amino-naphthalen-2-yl)-2-(1,2,3,6- |
| | tetrahydro-pyridin-4-yl)-4-(2-trifluoromethyl- |
| | phenoxymethyl)-benzamide |
| 312 | Naphthalene-1-carboxylic acid 2-(1,2,3,6- |
| | tetrahydro-pyridin-4-yl)-4-(2-trifluoromethyl- |
| | phenoxymethyl)-benzyl ester |
| 313 | Naphthalene-2-carboxylic acid 2-(1,2,3,6- |
| | tetrahydro-pyridin-4-yl)-4-(2-trifluoromethyl- |
| | phenoxymethyl)-benzyl ester |
| 314 | 2-Trifluoromethyl-benzoic acid 2-(1,2,3,6- |
| | tetrahydro-pyridin-4-yl)-4-(2-trifluoromethyl- |
| | phenoxymethyl)-benzyl ester |
| 315 | Benzyloxy-acetic acid 2-(1,2,3,6-tetrahydro- |
| | pyridin-4-yl)-4-(2-trifluoromethyl- |
| | phenoxymethyl)-benzyl ester |
| 316 | Benzo[1,3]dioxole-5-carboxylic acid 2-(1,2,3,6- |
| | tetrahydro-pyridin-4-yl)-4-(2-trifluoromethyl- |
| | phenoxymethyl)-benzyl ester |
| 317 | Terephthalic acid 1-methyl ester 4-[2-(1,2,3,6- |
| | tetrahydro-pyridin-4-yl)-4-(2-trifluoromethyl- |
| | phenoxymethyl)-benzyl] ester |
| 318 | Carbonic acid naphthalen-1-yl ester 2-(1,2,3,6- |
| | tetrahydro-pyridin-4-yl)-4-(2-trifluoromethyl- |
| | phenoxymethyl)-benzyl ester |
| 319 | Carbonic acid naphthalen-2-yl ester 2-(1,2,3,6- |
| | tetrahydro-pyridin-4-yl)-4-(2-trifluoromethyl- |
| | phenoxymethyl)-benzyl ester |
| 320 | 4-[2-(Naphthalen-1-yloxymethyl)-5-(2- |
| | trifluoromethyl-phenoxymethyl)-phenyl]-1,2,3,6- |
| | tetrahydro-pyridine |
| 321 | 4-[2-(Naphthalen-2-yloxymethyl)-5-(2- |
| | trifluoromethyl-phenoxymethyl)-phenyl]-1,2,3,6- |
| | tetrahydro-pyridine |
| 322 | N-Naphthalen-1-yl-2-(1,2,3,6-tetrahydro- |
| | pyridin-4-yl)-4-(2-trifluoromethyl- |
| | phenoxymethyl)-benzamide |
| 323 | 4-[5-(2-Trifluoromethyl-phenoxymethyl)-2-(4- |
| | trifluoromethyl-phenoxymethyl)-phenyl]-1,2,3,6- |
| | tetrahydro-pyridine |
| 324 | 4-[5-(2-Trifluoromethyl-phenoxymethyl)-2-(3- |
| | trifluoromethyl-phenoxymethyl)-phenyl]-1,2,3,6- |
| | tetrahydro-pyridine |
| 325 | 4-[2-(Biphenyl-4-yloxymethyl)-5-(2- |
| | trifluoromethyl-phenoxymethyl)-phenyl]-1,2,3,6- |
| | tetrahydro-pyridine |
| 326 | 4-[2-([1,1′;3′,1′′]Terphenyl-4′-yloxymethyl)-5- |
| | (2-trifluoromethyl-phenoxymethyl)-phenyl]- |
| | 1,2,3,6-tetrahydro-pyridine |
| 327 | 5-[2-(1,2,3,6-Tetrahydro-pyridin-4-yl)-4-(2- |
| | trifluoromethyl-phenoxymethyl)-benzyloxy]- |
| | quinoline |
| 328 | 3-[2-(1,2,3,6-Tetrahydro-pyridin-4-yl)-4-(2- |
| | trifluoromethyl-phenoxymethyl)-benzyloxy]- |
| | benzole acid methyl ester |
| 329 | 4-[2-(1,2,3,6-Tetrahydro-pyridin-4-yl)-4-(2- |
| | trifluoromethyl-phenoxymethyl)-benzyloxy]- |
| | benzoic acid methyl ester |
| 330 | 5-[2-(1,2,3,6-Tetrahydro-pyridin-4-yl)-4-(2- |
| | trifluoromethyl-phenoxymethyl)-benzyloxy]- |
| | isophthalic acid dimethyl ester |
| 331 | 5-[2-(1,2,3,6-Tetrahydro-pyridin-4-yl)-4-(2- |
| | trifluoromethyl-phenoxymethyl)-benzyloxy]-3,4- |
| | dihydro-2H-naphthalen-1-one |
| 332 | 2-Methyl-5-[2-(1,2,3,6-tetrahydro-pyridin-4- |
| | yl)-4-(2-trifluoromethyl-phenoxymethyl)- |
| | benzyloxy]-1H-indole-3-carboxylic acid ethyl |
| | ester |
| 333 | 4-[4-Bromo-2,5-bis-(2-trifluoromethyl- |
| | phenoxymethyl)-phenyl]-1,2,3,6-tetrahydro- |
| | pyridine |
| 334 | 4-[4-(1,2,3,6-Tetrahydro-pyridin-4-yl)-2,5-bis- |
| | (2-trifluoromethyl-phenoxymethyl)-phenyl]- |
| | 1,2,3,6-tetrahydro-pyridine |
| 335 | 4-[3′,4′-Dichloro-2,5-bis-(2-trifluoromethyl- |
| | phenoxymethyl)-biphenyl-4-yl]-1,2,3,6- |
| | tetrahydro-pyridine |
| 336 | 4-[2′-Trifluoromethyl-2,5-bis-(2- |
| | trifluoromethyl-phenoxymethyl)-biphenyl-4-yl]- |
| | 1,2,3,6-tetrahydro-pyridine |
| 337 | 4-[3′-Trifluoromethyl-2,5-bis-(2- |
| | trifluoromethyl-phenoxymethyl)-biphenyl-4-yl]- |
| | 1,2,3,6-tetrahydro-pyridine |
| 338 | 4-[4′-Trifluoromethyl-2,5-bis-(2- |
| | trifluoromethyl-phenoxymethyl)-biphenyl-4-yl]- |
| | 1,2,3,6-tetrahydro-pyridine |
| 339 | 4-[4-Naphthalen-2-yl-2,5-bis-(2- |
| | trifluoromethyl-phenoxymethyl)-phenyl]-1,2,3,6- |
| | tetrahydro-pyridine |
| 340 | 3-[4-(1,2,3,6-Tetrahydro-pyridin-4-yl)-2,5-bis- |
| | (2-trifluoromethyl-phenoxymethyl)-phenyl]- |
| | pyridine |
| 341 | 4-[4-(1,2,3,6-Tetrahydro-pyridin-4-yl)-2,5-bis- |
| | (2-trifluoromethyl-phenoxymethyl)-phenyl]- |
| | pyridine |
| 342 | 4-[4-Thiophen-3-yl-2,5-bis-(2-trifluoromethyl- |
| | phenoxymethyl)-phenyl]-1,2,3,6-tetrahydro- |
| | pyridine |
| 343 | 4-[4-Furan-3-yl-2,5-bis-(2-trifluoromethyl- |
| | phenoxymethyl)-phenyl]-1,2,3,6-tetrahydro- |
| | pyridine |
| 344 | 4-[2′-Nitro-2,5-bis-(2-trifluoromethyl- |
| | phenoxymethyl)-biphenyl-4-yl]-1,2,3,6- |
| | tetrahydro-pyridine |
| 345 | 4-[4-Thiophen-2-yl-2,5-bis-(2-trifluoromethyl- |
| | phenoxymethyl)-phenyl]-1,2,3,6-tetrahydro- |
| | pyridine |
| 346 | 4-[4-Furan-2-yl-2,5-bis-(2-trifluoromethyl- |
| | phenoxymethyl)-phenyl]-1,2,3,6-tetrahydro- |
| | pyridine |
| 347 | 4-[2′-Fluoro-2,5-bis-(2-trifluoromethyl- |
| | phenoxymethyl)-biphenyl-4-yl]-1,2,3,6- |
| | tetrahydro-pyridine |
| 348 | 4-[2′-Chloro-2,5-bis-(2-trifluoromethyl- |
| | phenoxymethyl)-biphenyl-4-yl]-1,2,3,6- |
| | tetrahydro-pyridine |
| 349 | 4-[2′,6′-Difluoro-2,5-bis-(2-trifluoromethyl- |
| | phenoxymethyl)-biphenyl-4-yl]-1,2,3,6- |
| | tetrahydro-pyridine |
| 350 | 1-[4′-(1,2,3,6-Tetrahydro-pyridin-4-yl)-2′,5′- |
| | bis-(2-trifluoromethyl-phenoxymethyl)-biphenyl- |
| | 2-yl]-ethanone |
| 351 | 4′-(1,2,3,6-Tetrahydro-pyridin-4-yl)-2′,5′-bis- |
| | (2-trifluoromethyl-phenoxymethyl)-biphenyl-3-ol |
| 352 | 4′-(1,2,3,6-Tetrahydro-pyridin-4-yl)-2′,5′-bis- |
| | (2-trifluoromethyl-phenoxymethyl)-biphenyl-4-ol |
| 353 | 4-[3′-Nitro-2,5-bis-(2-trifluoromethyl- |
| | phenoxymethyl)-biphenyl-4-yl]-1,2,3,6- |
| | tetrahydro-pyridine |
| 354 | 4-[4′-Nitro-2,5-bis-(2-trifluoromethyl- |
| | phenoxymethyl)-biphenyl-4-yl]-1,2,3,6- |
| | tetrahydro-pyridine |
| 355 | 1-[4′-(1,2,3,6-Tetrahydro-pyridin-4-yl)-2′,5′- |
| | bis-(2-trifluoromethyl-phenoxymethyl)-biphenyl- |
| | 2-yl]-ethanol |
| 356 | 4-[2,4,5-Tris-(2-trifluoromethyl- |
| | phenoxymethyl)-phenyl]-1,2,3,6-tetrahydro- |
| | pyridine |
| 357 | 4-[4-Benzofuran-2-yl-2,5-bis-(2- |
| | trifluoromethyl-phenoxymethyl)-phenyl]-1,2,3,6- |
| | tetrahydro-pyridine |
| 358 | 4-[4-(1H-Pyrrol-2-yl)-2,5-bis-(2- |
| | trifluoromethyl-phenoxymethyl)-phenyl]-1,2,3,6- |
| | tetrahydro-pyridine |
| 359 | 4′-(1,2,3,6-Tetrahydro-pyridin-4-yl)-2′,5′-bis- |
| | (2-trifluoromethyl-phenoxymethyl)-biphenyl-4- |
| | ylamine |
| 360 | 4-[3-(2-Trifluoromethyl-phenoxymethyl)- |
| | biphenyl-4-yl]-1,2,3,6-tetrahydro-pyridine |
| 361 | 4′-(1,2,3,6-Tetrahydro-pyridin-4-yl)-3′-(2- |
| | trifluoromethyl-phenoxymethyl)-biphenyl-4-ol |
| 362 | 4′-(1,2,3,6-Tetrahydro-pyridin-4-yl)-3′-(2- |
| | trifluoromethyl-phenoxymethyl)-biphenyl-2-ol |
| 363 | 4-[4-Furan-3-yl-2-(2-trifluoromethyl- |
| | phenoxymethyl)-phenyl]-1,2,3,6-tetrahydro- |
| | pyridine |
| 364 | 4′-(1,2,3,6-Tetrahydro-pyridin-4-yl)-2′,5′-bis- |
| | (2-trifluoromethyl-phenoxymethyl)-biphenyl-3- |
| | carboxylic acid amide |
| 365 | 4-[4′-Methoxy-2,5-bis-(2-trifluoromethyl- |
| | phenoxymethyl)-biphenyl-4-yl]-1,2,3,6- |
| | tetrahydro-pyridine |
| 366 | [4′-(1,2,3,6-Tetrahydro-pyridin-4-yl)-2′,5′- |
| | bis-(2-trifluoromethyl-phenoxymethyl)-biphenyl- |
| | 4-yl]-methanol |
| 367 | [4′-(1,2,3,6-Tetrahydro-pyridin-4-yl)-2′,5′- |
| | bis-(2-trifluoromethyl-phenoxymethyl)-biphenyl- |
| | 2-yl]-methanol |
| 368 | 4′-(1,2,3,6-Tetrahydro-pyridin-4-yl)-2′,5′-bis- |
| | (2-trifluoromethyl-phenoxymethyl)-biphenyl-3- |
| | carboxylic acid methyl ester |
| 369 | 4′-(1,2,3,6-Tetrahydro-pyridin-4-yl)-2′,5′-bis- |
| | (2-trifluoromethyl-phenoxymethyl)-biphenyl-4- |
| | carboxylic acid methyl ester |
| 370 | Furan-2-carboxylic acid 4′-(1,2,3,6-tetrahydro- |
| | pyridin-4-yl)-2′,5′-bis-(2-trifluoromethyl- |
| | phenoxymethyl)-biphenyl-2-ylmethyl ester |
| 371 | 4-[4-(1,2,3,6-Tetrahydro-pyridin-4-yl)-2-(2- |
| | trifluoromethyl-phenoxymethyl)-phenyl]-1,2,5,6- |
| | tetrahydro-pyridine |
| 372 | 4-[2′-Fluoro-3-(2-trifluoromethyl- |
| | phenoxymethyl)-biphenyl-4-yl]-1,2,3,6- |
| | tetrahydro-pyridine |
| 373 | 4-[2′-Chloro-3-(2-trifluoromethyl- |
| | phenoxymethyl)-biphenyl-4-yl]-1,2,3,6- |
| | tetrahydro-pyridine |
| 374 | 4-[2′-Methyl-3-(2-trifluoromethyl- |
| | phenoxymethyl)-biphenyl-4-yl]-1,2,3,6- |
| | tetrahydro-pyridine |
| 375 | 4-[2′-Trifluoromethyl-3-(2-trifluoromethyl- |
| | phenoxymethyl)-biphenyl-4-yl]-1,2,3,6- |
| | tetrahydro-pyridine |
| 376 | 4′-(1,2,3,6-Tetrahydro-pyridin-4-yl)-3′-(2- |
| | trifluoromethyl-phenoxymethyl)-biphenyl-2- |
| | ylamine |
| 377 | 4-[4-Bromo-2-(2-trifluoromethyl-phenoxymethyl)- |
| | phenyl]-1,2,3,6-tetrahydro-pyridine |
| 378 | [4′-(1,2,3,6-Tetrahydro-pyridin-4-yl)-3′-(2- |
| | trifluoromethyl-phenoxymethyl)-biphenyl-2-yl]- |
| | methanol |
| 379 | Benzoic acid 4′-(1,2,3,6-tetrahydro-pyridin-4- |
| | yl)-3′-(2-trifluoromethyl-phenoxymethyl)- |
| | biphenyl-2-yl methyl ester |
| 380 | 2-Trifluoromethyl-benzoic acid 4′-(1,2,3,6- |
| | tetrahydro-pyridin-4-yl)-3′-(2-trifluoromethyl- |
| | phenoxymethyl)-biphenyl-2-ylmethyl ester |
| 381 | 2-Bromo-5-(1,2,3,6-tetrahydro-pyridin-4-yl)-4- |
| | (2-trifluoromethyl-phenoxymethyl)-benzoic acid |
| | methyl ester |
| 382 | 2,5-Bis-(1,2,3,6-tetrahydro-pyridin-4-yl)-4-(2- |
| | trifluoromethyl-phenoxymethyl)-benzoic acid |
| | methyl ester |
| 383 | 2-Furan-3-yl-5-(1,2,3,6-tetrahydro-pyridin-4- |
| | yl)-4-(2-trifluoromethyl-phenoxymethyl)-benzoic |
| | acid methyl ester |
| 384 | 2-Chloro-nicotinic acid 4′-(1,2,3,6-tetrahydro- |
| | pyridin-4-yl)-3′-(2-trifluoromethyl- |
| | phenoxymethyl)-biphenyl-2-ylmethyl ester |
| 385 | Nicotinic acid 2-furan-3-yl-5-(1,2,3,6- |
| | tetrahydro-pyridin-4-yl)-4-(2-trifluoromethyl- |
| | phenoxymethyl)-benzyl ester |
| 386 | 2-Chloro-nicotinic acid 2-furan-3-yl-5- |
| | (1,2,3,6-tetrahydro-pyridin-4-yl)-4-(2- |
| | trifluoromethyl-phenoxymethyl)-benzyl ester |
| 387 | [2-Furan-3-yl-5-(1,2,3,6-tetrahydro-pyridin-4- |
| | yl)-4-(2-trifluoromethyl-phenoxymethyl)- |
| | phenyl]-methanol |
| 388 | [2-Furan-3-yl-5-(1-methyl-1,2,3,6-tetrahydro- |
| | pyridin-4-yl)-4-(2-trifluoromethyl- |
| | phenoxymethyl)-phenyl]-methanol |
| 389 | Pyridine-2-carboxylic acid 2-furan-3-yl-5- |
| | (1,2,3,6-tetrahydro-pyridin-4-yl)-4-(2- |
| | trifluoromethyl-phenoxymethyl)-benzyl ester |
| 390 | Isonicotinic acid 2-furan-3-yl-5-(1,2,3,6- |
| | tetrahydro-pyridin-4-yl)-4-(2-trifluoromethyl- |
| | phenoxymethyl)-benzyl ester |

According to another preferred embodiment, the present invention provides a method of inhibiting BACE activity in a mammal, comprising the step of administering to said mammal a compound of formula IAB:

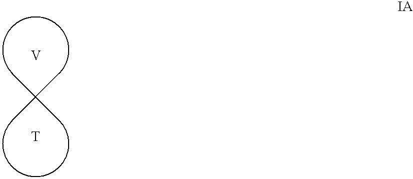

wherein:

V is selected from IA1, IB1, IB2, IB4, IB5, or IB6;

T is a five to eleven membered monocyclic or bicyclic, aromatic or non-aromatic ring having zero to three heteroatoms independently selected from O, S, N or NH, wherein T has at least one R¹⁰ substituent and up to three more substituents selected from R¹⁰ or J;

T and V share a ring atom;

J is halogen, —R′, —OR′, —NO₂, —CN, —CF₃, —OCF₃, oxo, 1,2-methylenedioxy, —N(R′)₂, —SR′, —S(O)R′, —S(O)N(R′)₂, —SO₂R′, —C(O)R′, —CO₂R′, —C(O)N(R′)₂, —N(R′)C(O)R′, —N(R′)C(O)OR′, —N(R′)C(O)N(R′)₂, or —OC(O)N(R′)₂, wherein R′ is independently selected from hydrogen, aliphatic, heterocyclyl, heterocycly-alkyl, aryl, aralkyl, heteroaryl, or heteroaralkyl;

wherein R′ is optionally substituted with up to 3 substituents selected independently from —R¹¹, —OR¹¹, —NO₂, —CN, —CF₃, —OCF₃, oxo, 1,2-methylenedioxy, —N(R¹¹)₂, —SR¹¹, —S(O)R¹¹, —S(O)N(R¹¹)₂, —SO₂R¹¹, —C(O)R11, —CO₂R¹¹, —C(O)N(R¹¹)₂, —N(R¹¹)C(O)R′, —N(R¹¹)C(O)OR¹¹, —N(R¹¹)C(O)N(R¹¹)₂, or —OC(O)N(R¹¹)₂,;

R¹¹ is hydrogen, (C₁-C₆)-alkyl, (C₂-C₆)-alkenyl or alkynyl, or (C₃-C₆)cycloalkyl;

R¹⁰ is P1-R1-P2-R2-W;

P1 and P2 each are independently:

absent; or

aliphatic;

R1 and R2 each are independently:

absent; or

R;

R is a suitable linker;

W is five to eleven membered monocyclic or bicyclic, aromatic or non-aromatic ring having zero to three heteroatoms independently selected from O, S, N, or NH, wherein W has up to 3 J substituents.

According to another preferred embodiment, the compound of formula IA is selected from formula ICa or formula ICb:

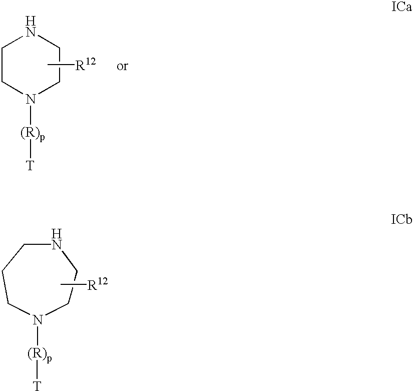

or a pharmaceutically acceptable salt thereof, wherein:

R is a suitable linker;

p is zero or one;

R¹² is absent or R¹⁰;

R¹⁰ is P1-R1-P2-R2-W;

T is a five to eleven membered monocyclic or bicyclic, aromatic or non-aromatic ring having zero to three heteroatoms independently selected from O, S, N or NH, wherein T has at least one R¹⁰ substituent and up to three more substituents selected from R¹⁰ or J;

J is halogen, —R′, —OR′, —NO₂, —CN, —CF₃, —OCF₃, oxo, 1,2-methylenedioxy, —N(R′)₂, —SR′, —SOR′, —SO₂R′, —C(O)R′, —C(O)OR′ or —C(O)N(R′)₂, wherein R′ is independently selected from hydrogen, aliphatic, heterocyclyl, heterocyclyl-alkyl, aryl, aralkyl, heteroaryl, or heteroaralkyl;

P1 and P2 each are independently:

absent; or

aliphatic;

R1 and R2 each are independently:

absent; or

R;

W is five to eleven membered monocyclic or bicyclic, aromatic or non-aromatic ring having zero to three heteroatoms independently selected from O, S, N, or NH, wherein W has up to 3 substituents independently selected from J.

Preferred embodiments of V in formula ICa and ICb are as shown below:

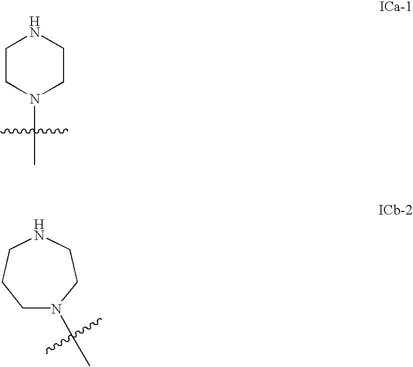

According to a more preferred embodiment of formula ICa and ICb, V is ICa-1.

Representative compounds of formulae ICa and ICb are listed below in Table 3.

TABLE 3
Compounds of Formulae ICa and ICb

| | |
|---|---|
| 100 | Naphthalen-2-ylmethyl-(2-piperazin-1-yl-5- |
| | trifluoromethyl-phenyl)-amine |
| 101 | 4-Fluoro-naphthalene-1-carboxylic acid (2- |
| | piperazin-1-yl-5-trifluoromethyl-phenyl)-amide |
| 102 | Isoquinoline-1-carboxylic acid (2-piperazin-1-yl- |
| | 5-trifluoromethyl-phenyl)-amide |
| 103 | Naphthalene-1-carboxylic acid (4′-fluoro-4- |
| | piperazin-1-yl-biphenyl-3-yl)-amide |
| 104 | Naphthalene-1-carboxylic acid (3′-chloro-4′- |
| | fluoro-4-piperazin-1-yl-biphenyl-3-yl)-amide |
| 105 | Naphthalene-1-carboxylic acid (4′-fluoro-3′- |
| | formyl-4-piperazin-1-yl-biphenyl-3-yl)-amide |
| 106 | Naphthalene-1-carboxylic acid (2′,3′-dichloro-4- |
| | piperazin-1-yl-biphenyl-3-yl)-amide |
| 107 | Naphthalene-1-carboxylic acid (2′,4′-dichloro-4- |
| | piperazin-1-yl-biphenyl-3-yl)-amide |
| 108 | Naphthalene-1-carboxylic acid (2′,5′-dichloro-4- |
| | piperazin-1-yl-biphenyl-3-yl)-amide |
| 109 | Naphthalene-1-carboxylic acid (2′,3′,5′- |
| | trichloro-4-piperazin-1-yl-biphenyl-3-yl)-amide |
| 110 | Naphthalene-1-carboxylic acid (2-piperazin-1-yl- |
| | 5-pyridin-3-yl-phenyl)-amide |
| 111 | Naphthalene-1-carboxylic acid (2-piperazin-1-yl- |
| | 5-pyridin-4-yl-phenyl)-amide |
| 112 | Naphthalene-1-carboxylic acid (5-bromo-4-methyl- |
| | 2-piperazin-1-yl-phenyl)-amide |
| 113 | Naphthalene-2-carboxylic acid (2-piperazin-1-yl- |
| | 5-trifluoromethyl-phenyl)-amide |
| 114 | Naphthalene-1-carboxylic acid (2-piperazin-1-yl- |
| | 5-trifluoromethyl-phenyl)-amide |
| 115 | 4-{2,6-Bis-[(naphthalene-2-carbonyl)-amino]-4- |
| | trifluoromethyl-phenyl}-piperazine |
| 116 | 1-[2,5-Bis-(2-trifluoromethyl-phenoxymethyl)- |
| | phenyl]-piperazine |
| 117 | 4-tert-Butyl-N-(2-piperazin-1-yl-5- |
| | trifluoromethyl-phenyl)-benzamide |
| 118 | Naphthalene-1-carboxylic acid (5-bromo-2- |
| | piperazin-1-yl-phenyl)-amide |
| 119 | Naphthalene-1-carboxylic acid (3′-methoxy-4- |
| | piperazin-1-yl-biphenyl-3-yl)-amide |
| 120 | Naphthalene-1-carboxylic acid (4′-methoxy-4- |
| | piperazin-1-yl-biphenyl-3-yl)-amide |
| 121 | Naphthalene-1-carboxylic acid (4′-chloro-4- |
| | piperazin-1-yl-biphenyl-3-yl)-amide |
| 122 | Naphthalene-1-carboxylic acid (2′-chloro-4- |
| | piperazin-1-yl-biphenyl-3-yl)-amide |
| 123 | Naphthalene-1-carboxylic acid (3′-chloro-4- |
| | piperazin-1-yl-biphenyl-3-yl)-amide |
| 124 | Naphthalene-1-carboxylic acid (4′-methyl-4- |
| | piperazin-1-yl-biphenyl-3-yl)-amide |
| 125 | Naphthalene-1-carboxylic acid [2-piperazin-1-yl- |
| | 5-(2-trifluoromethyl-phenoxymethyl)-phenyl]-amide |
| 126 | Naphthalene-1-carboxylic acid (3′-methyl-4- |
| | piperazin-1-yl-biphenyl-3-yl)-amide |
| 127 | 4-{2,6-Bis-[(naphthalene-1-carbonyl)-amino]-4- |
| | trifluoromethyl-phenyl}-piperazine |
| 128 | Naphthalene-1-carboxylic acid (4-piperazin-1-yl- |
| | 3′-trifluoromethyl-biphenyl-3-yl)-amide |
| 129 | Naphthalene-1-carboxylic acid (4-piperazin-1-yl- |
| | 4′-trifluoromethyl-biphenyl-3-yl)-amide |
| 130 | Naphthalene-1-carboxylic acid (3′,4′-dichloro-4- |
| | piperazin-1-yl-biphenyl-3-yl)-amide |
| 131 | Naphthalene-1-carboxylic acid (4′-cyano-4- |
| | piperazin-1-yl-biphenyl-3-yl)-amide |
| 132 | Naphthalene-1-carboxylic acid (5-phenoxy-2- |
| | piperazin-1-yl-phenyl)-amide |
| 133 | Naphthalene-1-carboxylic acid [5-(4-chloro- |
| | phenoxy)-2-piperazin-1-yl-phenyl]-amide |
| 134 | 2-Naphthalen-1-yl-N-(2-piperazin-1-yl-5- |
| | trifluoromethyl-phenyl)-acetamide |
| 135 | Naphthalene-1-sulfonic acid (2-piperazin-1-yl-5- |
| | trifluoromethyl-phenyl)-amide |
| 136 | Naphthalene-2-sulfonic acid (2-piperazin-1-yl-5- |
| | trifluoromethyl-phenyl)-amide |
| 137 | Biphenyl-4-sulfonic acid (2-piperazin-1-yl-5- |
| | trifluoromethyl-phenyl)-amide |
| 138 | Naphthalene-1-carboxylic acid (3′,4′-dichloro-6- |
| | methyl-4-piperazin-1-yl-biphenyl-3-yl)-amide |
| 139 | Naphthalene-1-carboxylic acid [5-(3-chloro- |
| | phenoxy)-2-piperazin-1-yl-phenyl]-amide |
| 140 | Naphthalene-1-carboxylic acid (2-piperazin-1-yl- |
| | 5-o-tolyloxy-phenyl)-amide |
| 141 | Naphthalene-1-carboxylic acid (2-piperazin-1-yl- |
| | 5-m-tolyloxy-phenyl)-amide |
| 142 | Naphthalene-1-carboxylic acid (2-piperazin-1-yl- |
| | 5-p-tolyloxy-phenyl)-amide |
| 143 | 6-Methoxy-naphthalene-1-carboxylic acid (2- |
| | piperazin-1-yl-5-trifluoromethyl-phenyl)-amide |
| 144 | Naphthalene-1-carboxylic acid (4′- |
| | isopropylsulfamoyl-4-piperazin-1-yl-biphenyl-3- |
| | yl)-amide |
| 145 | Naphthalene-1-carboxylic acid (4′- |
| | diethylsulfamoyl-4-piperazin-1-yl-biphenyl-3-yl)- |
| | amide |
| 146 | Naphthalene-1-carboxylic acid (4′- |
| | benzylsulfamoyl-4-piperazin-1-yl-biphenyl-3-yl)- |
| | amide |
| 147 | Naphthalene-1-carboxylic acid (4′- |
| | cyclohexylsulfamoyl-4-piperazin-1-yl-biphenyl-3- |
| | yl)-amide |
| 148 | Naphthalene-1-carboxylic acid (3-chloro-2- |
| | piperazin-1-yl-5-trifluoromethyl-phenyl)-amide |
| 149 | Quinoline-8-carboxylic acid (2-piperazin-1-yl-5- |
| | trifluoromethyl-phenyl)-amide |
| 150 | (2-Piperazin-1-yl-5-trifluoromethyl-phenyl)- |
| | carbamic acid naphthalen-1-yl ester |
| 151 | (2-Piperazin-1-yl-5-trifluoromethyl-phenyl)- |
| | carbamic acid naphthalen-2-yl ester |
| 152 | Naphthalene-1-carboxylic acid (5-furan-3-yl-2- |
| | piperazin-1-yl-phenyl)-amide |
| 153 | Naphthalene-1-carboxylic acid (2-piperazin-1-yl- |
| | 5-thiophen-3-yl-phenyl)-amide |
| 154 | Naphthalene-1-carboxylic acid (5-furan-3-yl-4- |
| | methyl-2-piperazin-1-yl-phenyl)-amide |
| 155 | Naphthalene-1-carboxylic acid (4-methyl-2- |
| | piperazin-1-yl-5-thiophen-3-yl-phenyl)-amide |
| 156 | Naphthalene-1-carboxylic acid (4-benzyloxy-2- |
| | piperazin-1-yl-phenyl)-amide |
| 157 | Naphthalene-1-carboxylic acid (4-bromo-5-fluoro- |
| | 2-piperazin-1-yl-phenyl)-amide |
| 158 | Naphthalene-1-carboxylic acid (2-fluoro-5- |
| | piperazin-1-yl-biphenyl-4-yl)-amide |
| 159 | Naphthalene-1-carboxylic acid (2-fluoro-5- |
| | piperazin-1-yl-4′-trifluoromethyl-biphenyl-4-yl)- |
| | amide |
| 160 | Naphthalene-1-carboxylic acid (5-fluoro-4-furan- |
| | 3-yl-2-piperazin-1-yl-phenyl)-amide |
| 161 | Naphthalene-1-carboxylic acid (2′-fluoro-4- |
| | piperazin-1-yl-4′-trifluoromethyl-biphenyl-3-yl)- |
| | amide |
| 162 | Naphthalene-1-carboxylic acid (2′,5′-difluoro-4- |
| | piperazin-1-yl-4′-trifluoromethyl-biphenyl-3-yl)- |
| | amide |
| 163 | Naphthalene-1-carboxylic acid (4′- |
| | benzylsulfamoyl-3′-fluoro-4-piperazin-1-yl- |
| | biphenyl-3-yl)-amide |
| 164 | Naphthalene-1-carboxylic acid (4′- |
| | benzylsulfamoyl-2′,5′-difluoro-4-piperazin-1-yl- |
| | biphenyl-3-yl)-amide |
| 165 | Naphthalen-2-ylmethyl-(4-piperazin-1-yl-biphenyl- |
| | 3-yl)-amine |
| 166 | Naphthalen-2-ylmethyl-(4-piperazin-1-yl-4′- |
| | trifluoromethyl-biphenyl-3-yl)-amine |
| 167 | Naphthalene-1-carboxylic acid (4-chloro-2- |
| | piperazin-1-yl-5-trifluoromethyl-phenyl)-amide |
| 168 | Naphthalene-1-carboxylic acid (3′,4′-dichloro-5- |
| | piperazin-1-yl-2-trifluoromethyl-biphenyl-4-yl)- |
| | amide |
| 169 | Naphthalene-1-carboxylic acid (2′,5′-dichloro-5- |
| | piperazin-1-yl-2-trifluoromethyl-biphenyl-4-yl)- |
| | amide |
| 170 | Naphthalene-1-carboxylic acid (5-piperazin-1-yl- |
| | 2,4′-bis-trifluoromethyl-biphenyl-4-yl)-amide |
| 171 | 4′-Trifluoromethyl-biphenyl-4-sulfonic acid (2- |
| | piperazin-1-yl-5-trifluoromethyl-phenyl)-amide |
| 172 | 2′-Trifluoromethyl-biphenyl-4-sulfonic acid (2- |
| | piperazin-1-yl-5-trifluoromethyl-phenyl)-amide |
| 173 | Naphthalene-1-carboxylic acid (3′,4′-dichloro-3- |
| | piperazin-1-yl-biphenyl-4-yl)-amide |
| 174 | Naphthalene-1-carboxylic acid (3-piperazin-1-yl- |
| | 4′-trifluoromethyl-biphenyl-4-yl)-amide |
| 175 | Naphthalene-1-carboxylic acid (3′,4′-dichloro-2- |
| | fluoro-5-piperazin-1-yl-biphenyl-4-yl)-amide |
| 176 | Isoquinoline-1-carboxylic acid [5-bromo-2- |
| | piperazin-1-yl-3-(2-trifluoromethyl- |
| | phenoxymethyl)-phenyl]-amide |
| 177 | Isoquinoline-1-carboxylic acid [4-piperazin-1-yl- |
| | 5-(2-trifluoromethyl-phenoxymethyl)-biphenyl-3- |
| | yl]-amide |
| 178 | Isoquinoline-1-carboxylic acid [2-piperazin-1-yl- |
| | 4-(2-trifluoromethyl-phenoxymethyl)-phenyl]-amide |
| 179 | 4′-Trifluoromethyl-biphenyl-4-sulfonic acid |
| | (3′,4′-dichloro-4-piperazin-1-yl-biphenyl-3-yl)- |
| | amide |
| 180 | 3′-Chloro-biphenyl-4-sulfonic acid (3′,4′- |
| | dichloro-4-piperazin-1-yl-biphenyl-3-yl)-amide |
| 181 | 4′-Chloro-biphenyl-4-sulfonic acid (3′,4′- |
| | dichloro-4-piperazin-1-yl-biphenyl-3-yl)-amide |
| 182 | 3′-Methyl-biphenyl-4-sulfonic acid (3′,4′- |
| | dichloro-4-piperazin-1-yl-biphenyl-3-yl)-amide |
| 182 | 4′-Methyl-biphenyl-4-sulfonic acid (3′,4′- |
| | dichloro-4-piperazin-1-yl-biphenyl-3-yl)-amide |
| 183 | Isoquinoline-1-carboxylic acid [5-bromo-2- |
| | piperazin-1-yl-4-(2-trifluoromethyl- |
| | phenoxymethyl)-phenyl]-amide |
| 184 | Isoquinoline-1-carboxylic acid [4-piperazin-1-yl- |
| | 6-(2-trifluoromethyl-phenoxymethyl)-biphenyl-3- |
| | yl]-amide |
| 185 | Isoquinoline-1-carboxylic acid [4-piperazin-1-yl- |
| | 4′-trifluoromethyl-6-(2-trifluoromethyl- |
| | phenoxymethyl)-biphenyl-3-yl]-amide |
| 186 | Isoquinoline-1-carboxylic acid [4′-hydroxy-4- |
| | piperazin-1-yl-6-(2-trifluoromethyl- |
| | phenoxymethyl)-biphenyl-3-yl]-amide |
| 187 | Isoquinoline-1-carboxylic acid [5-furan-3-yl-2- |
| | piperazin-1-yl-4-(2-trifluoromethyl- |
| | phenoxymethyl)-phenyl]-amide |
| 188 | 5-Bromo-2-piperazin-1-yl-3-[(quinolin-2- |
| | ylmethyl)-amino]-benzoic acid ethyl ester |
| 189 | Quinoxaline-2-carboxylic acid (2-piperazin-1-yl- |
| | 5-trifluoromethyl-phenyl)-amide |
| 190 | [1,6]Naphthyridine-2-carboxylic acid (2- |
| | piperazin-1-yl-5-trifluoromethyl-phenyl)-amide |
| 191 | 4-{3-[(Naphthalen-2-ylmethyl)-amino]-4′- |
| | trifluoromethyl-biphenyl-4-yl}-piperazine-2- |
| | carboxylic acid |
| 192 | 4-{3-[(Naphthalen-2-ylmethyl)-amino]-4′- |
| | trifluoromethyl-biphenyl-4-yl}-piperazine-2- |
| | carboxylic acid methyl ester |
| 193 | 4-{3-[(Naphthalen-2-ylmethyl)-amino]-4′- |
| | trifluoromethyl-biphenyl-4-yl}-piperazine-2- |
| | carboxylic acid isopropylamide |
| 194 | 4-{3-[(Naphthalen-2-ylmethyl)-amino]-4′- |
| | trifluoromethyl-biphenyl-4-yl}-piperazine-2- |
| | carboxylic acid benzylamide |
| 195 | 4-{3-[(Naphthalen-2-ylmethyl)-amino]-4′- |
| | trifluoromethyl-biphenyl-4-yl}-piperazine-2- |
| | carboxylic acid dimethylamide |
| 196 | Naphthalene-1-carboxylic acid [6-(3,4-dichloro- |
| | phenyl)-2-piperazin-1-yl-pyridin-3-yl]-amide |
| 197 | Naphthalene-1-carboxylic acid [2-(3,4-dichloro- |
| | phenyl)-4-piperazin-1-yl-pyrimidin-5-yl]-amide |
| 200 | 1-[4-(4-Chloro-2-methyl-phenoxy)-butyryl]- |
| | piperazine-2-carboxylic acid naphthalen-2-ylamide |
| 201 | Naphthalene-1-carboxylic acid (2-[1,4]diazepan-1- |
| | yl-5-trifluoromethyl-phenyl)-amide |
| 206 | 1-[3-(2-Trifluoromethyl-phenoxymethyl)-benzoyl]- |
| | piperazine-2-carboxylic acid naphthalen-2-ylamide |
| 217 | Naphthalene-1-carboxylic acid [2-(3,4-dichloro- |
| | phenyl)-4-piperazin-1-yl-pyrimidin-5-yl]-amide |
| 227 | Naphthalene-1-carboxylic acid [6-(3,4-dichloro- |
| | phenyl)-2-piperazin-1-yl-pyridin-3-yl]-amide |

Each of the preferred embodiment of V, recited above, may be combined with any of the preferred embodiments of R, p and T, recited above, to produce a preferred embodiment of compound of formula (IA).

According to another embodiment, the present invention provides a method of inhibiting BACE activity in a mammal, comprising the step of administering to said mammal a compound of formula ID:

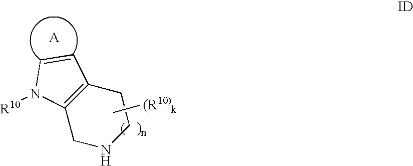

or a pharmaceutically acceptable salt thereof, wherein:

A is a five or six membered aryl ring having zero to two heteroatoms independently selected from nitrogen, oxygen or sulfur, wherein:

A has at least one R¹⁰ substituent and up to three more substituents selected from R¹⁰ or J;

k is 0 or 1;

n is 0-2;

J is halogen, —R′, —OR′, —NO₂, —CN, —CF₃, —OCF₃, oxo, 1,2-methylenedioxy, —N(R′)₂, —SR′, —S(O)R′, —S(O)N(R′)₂, —SO₂R′, —C(O)R′, —CO₂R′, —C(O)N(R′)₂, —N(R′)C(O)R′, —N(R′)C(O)OR′, —N(R′)C(O)N(R′)₂, or —OC(O)N(R′)₂, wherein R′ is independently selected from hydrogen, aliphatic, heterocyclyl, heterocycly-alkyl, aryl, aralkyl, heteroaryl, or heteroaralkyl;

wherein R′ is optionally substituted with up to 3 substituents selected independently from —R¹¹, —OR¹¹, —NO₂, —CN, —CF₃, —OCF₃, oxo, 1,2-methylenedioxy, —N(R¹¹)₂, —SR¹¹, —S(O)R¹¹, —S(O)N(R¹)₂, —SO₂R¹¹, —C(O)R11, —CO₂R , —C(O)N(R¹¹)₂, —N(R¹¹)C(O)R′, —N(R¹¹)C(O)OR¹¹, —N(R¹¹)C(O)N(R¹¹)₂, or —OC(O)N(R¹¹)₂,;

R¹¹ is hydrogen, (C₁-C₆)-alkyl, (C₂-C₆)-alkenyl or alkynyl, or (C₃-C₆)cycloalkyl;

R¹⁰ is P1-R1-P2-R2-W;

P1 and P2 each are independently:

absent; or

aliphatic;

R1 and R2 each are independently:

absent; or

R;

R is a suitable linker;

W is a five to eleven membered monocyclic or bicyclic, aromatic or non-aromatic ring having zero to three heteroatoms independently selected from O, S, N, or NH, wherein W has up to 3 substituents independently selected from J.

Preferred embodiments of R¹⁰ and R in compounds of formula ID are as recited above for R¹⁰ and R in compounds of formula IA.

More preferred compounds of formula ID are as shown below:

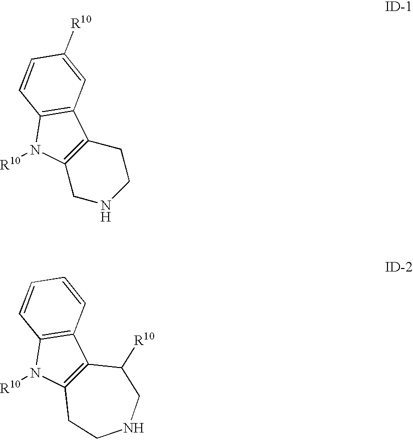

wherein R¹⁰ is as defined above.

Representative compounds of formula ID are listed below in Table 4.

TABLE 4
Compounds of Formula ID

| | |
|---|---|
| 202 | 1,2,3,4,5,6-Hexahydro-azepino[4,5-b]indole-5- |
| | carboxylic acid naphthalen-2-ylamide |
| 501 | 6-Benzyloxy-9-naphthalen-2-ylmethyl-2,3,4,9- |
| | tetrahydro-1H-b-carboline |
| 502 | (6-Methoxy-1,2,3,4-tetrahydro-b-carbolin-9-yl)- |
| | naphthalen-2-yl-methanone |
| 503 | 6-Methoxy-9-naphthalen-2-ylmethyl-2,3,4,9- |
| | tetrahydro-1H-b-carboline |
| 504 | Naphthalen-1-yl-[6-(2-trifluoromethyl- |
| | benzyloxy)-1,2,3,4-tetrahydro-b-carbolin-9-yl]- |
| | methanone |
| 505 | 9-Naphthalen-1-ylmethyl-6-(2-trifluoromethyl- |
| | benzyloxy)-2,3,4,9-tetrahydro-1H-b-carboline |
| 506 | Naphthalen-1-yl-[6-(4-trifluoromethyl- |
| | benzyloxy)-1,2,3,4-tetrahydro-b-carbolin-9-yl]- |
| | methanone |
| 507 | Naphthalen-2-yl-[6-(3-trifluoromethyl- |
| | benzyloxy)-1,2,3,4-tetrahydro-b-carbolin-9-yl]- |
| | methanone |
| 508 | Naphthalen-1-yl-[6-(3-trifluoromethyl- |
| | benzyloxy)-1,2,3,4-tetrahydro-b-carbolin-9-yl]- |
| | methanone |
| 509 | 9-Naphthalen-1-ylmethyl-6-(3-trifluoromethyl- |
| | benzyloxy)-2,3,4,9-tetrahydro-1H-b-carboline |
| 510 | [6-(2-Chloro-5-trifluoromethyl-benzyloxy)- |
| | 1,2,3,4-tetrahydro-b-carbolin-9-yl]-naphthalen- |
| | 1-yl-methanone |
| 511 | [6-(2-Chloro-5-trifluoromethyl-benzyloxy)- |
| | 1,2,3,4-tetrahydro-b-carbolin-9-yl]-naphthalen- |
| | 2-yl-methanone |
| 512 | 6-(4-Difluoromethoxy-benzyloxy)-9-naphthalen-1- |
| | ylmethyl-2,3,4,9-tetrahydro-1H-b-carboline |
| 513 | 6-(4-Difluoromethoxy-benzyloxy)-9-naphthalen-2- |
| | ylmethyl-2,3,4,9-tetrahydro-1H-b-carboline |
| 514 | [6-(4-Difluoromethoxy-benzyloxy)-1,2,3,4- |
| | tetrahydro-b-carbolin-9-yl]-naphthalen-1-yl- |
| | methanone |
| 515 | [6-(4-Difluoromethoxy-benzyloxy)-1,2,3,4- |
| | tetrahydro-b-carbolin-9-yl]-naphthalen-2-yl- |
| | methanone |
| 516 | 6-(2-Difluoromethoxy-benzyloxy)-9-naphthalen-2- |
| | ylmethyl-2,3,4,9-tetrahydro-1H-b-carboline |
| 517 | [6-(2,5-Bis-trifluoromethyl-benzyloxy)-1,2,3,4- |
| | tetrahydro-b-carbolin-9-yl]-naphthalen-1-yl- |
| | methanone |
| 518 | 6-(2-Difluoromethoxy-benzyloxy)-9-naphthalen-1- |
| | ylmethyl-2,3,4,9-tetrahydro-1H-b-carboline |
| 519 | 6-(Naphthalen-2-ylmethoxy)-9-naphthalen-2- |
| | ylmethyl-2,3,4,9-tetrahydro-1H-b-carboline |
| 520 | 6-(2-Iodo-benzyloxy)-9-naphthalen-1-ylmethyl- |
| | 2,3,4,9-tetrahydro-1H-b-carboline |
| 521 | 6-(2-Methyl-3-trifluoromethyl-benzyloxy)-9- |
| | naphthalen-1-ylmethyl-2,3,4,9-tetrahydro-1H-b- |
| | carboline |
| 522 | 6-(2-Methyl-3-trifluoromethyl-benzyloxy)-9- |
| | naphthalen-2-ylmethyl-2,3,4,9-tetrahydro-1H-b- |
| | carboline |
| 523 | [6-(2-Methyl-3-trifluoromethyl-benzyloxy)- |
| | 1,2,3,4-tetrahydro-b-carbolin-9-yl]-naphthalen- |
| | 1-yl-methanone |
| 524 | [6-(2-Methyl-3-trifluoromethyl-benzyloxy)- |
| | 1,2,3,4-tetrahydro-b-carbolin-9-yl]-naphthalen- |
| | 2-yl-methanone |
| 525 | 6-(3,5-Dimethoxy-benzyloxy)-9-naphthalen-1- |
| | ylmethyl-2,3,4,9-tetrahydro-1H-b-carboline |
| 526 | [6-(3,5-Dimethoxy-benzyloxy)-1,2,3,4- |
| | tetrahydro-b-carbolin-9-yl]-naphthalen-1-yl- |
| | methanone |
| 527 | [6-(3,5-Dimethoxy-benzyloxy)-1,2,3,4- |
| | tetrahydro-b-carbolin-9-yl]-naphthalen-2-yl- |
| | methanone |
| 528 | [6-(2-Iodo-benzyloxy)-1,2,3,4-tetrahydro-b- |
| | carbolin-9-yl]-naphthalen-1-yl-methanone |
| 529 | [6-(2-Difluoromethoxy-benzyloxy)-1,2,3,4- |
| | tetrahydro-b-carbolin-9-yl]-naphthalen-1-yl- |
| | methanone |
| 530 | [6-(2-Difluoromethoxy-benzyloxy)-1,2,3,4- |
| | tetrahydro-b-carbolin-9-yl]-naphthalen-2-yl- |
| | methanone |
| 531 | 4′-(9-Naphthalen-2-ylmethyl-2,3,4,9-tetrahydro- |
| | 1H-b-carbolin-6-yloxymethyl)-biphenyl-2- |
| | carbonitrile |
| 532 | 4′-[9-(Naphthalene-1-carbonyl)-2,3,4,9- |
| | tetrahydro-1H-b-carbolin-6-yloxymethyl]- |
| | biphenyl-2-carbonitrile |
| 533 | 9-Naphthalen-1-ylmethyl-6-(4-trifluoromethyl- |
| | benzyloxy)-2,3,4,9-tetrahydro-1H-b-carboline |
| 534 | 9-Naphthalen-2-ylmethyl-6-(4-trifluoromethyl- |
| | benzyloxy)-2,3,4,9-tetrahydro-1H-b-carboline |
| 535 | 9-Naphthalen-2-ylmethyl-6-(2-trifluoromethyl- |
| | benzyloxy)-2,3,4,9-tetrahydro-1H-b-carboline |
| 536 | Naphthalen-2-yl-[6-(4-trifluoromethyl- |
| | benzyloxy)-1,2,3,4-tetrahydro-b-carbolin-9-yl]- |
| | methanone |
| 537 | 9-Naphthalen-2-ylmethyl-6-(3-trifluoromethyl- |
| | benzyloxy)-2,3,4,9-tetrahydro-1H-b-carboline |

According to another embodiment, the present invention provides a method of inhibiting BACE activity in mammal, comprising the step of administering to said mammal a compound of formula IE:

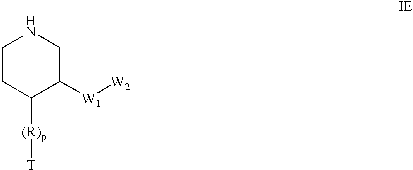

wherein:

W₁ is —NH—, —CH₂—NH—, —C(O)—NH—, or —C(O)—O—;

W₂ is P1-R1-P2-R2-W;

P1 and P2 each are independently:

absent; or

aliphatic;

R1 and R2 each are independently:

absent; or

R;

W is five to eleven membered monocyclic or bicyclic, aromatic or non-aromatic ring having zero to three heteroatoms independently selected from O, S, N, or NH, wherein W has up to 3 substituents independently selected from J;

R is —CH₂—, —O—, —S—, —SO—, —SO₂—, —NR′—, —C(O)O—, —OC(O)—, —C(O)NR′—, —NR′C.(O)—, —O—, —OC(O)NR′—, —NR′C(O)NR′—, —NR′C.(O)O—, —SO—NR′, —NR′SO—, —NR′SO₂—, —SO₂NR′—, —CHOR′—, —CHNR′—, or —C(O)—;

J is halogen, —R′, —OR′, —NO₂, —CN, —CF₃, —OCF₃, oxo, 1,2-methylenedioxy, —N(R′)₂, —SR′, —S(O)R′, —S(O)N(R′)₂, —SO₂R′, —C(O)R′, —CO₂R′ or —C(O)N(R′ )₂, wherein R′ is independently selected from hydrogen, aliphatic, heterocyclyl, heterocycly-alkyl, aryl, aralkyl, heteroaryl, or heteroaralkyl;

wherein R′ is optionally substituted with up to 3 substituents selected independently from —R¹¹, —OR¹¹, —NO₂, —CN, —CF₃, —OCF₃, oxo, 1,2-methylenedioxy, —N(R¹¹)₂, —S R¹¹, —S(O)R, —S(O)N(R¹¹)₂, —SO₂R¹¹, —C(O)R¹¹, —CO₂R¹¹ or —C(O)N(R¹¹)₂,;

R¹¹ is hydrogen, (C₁-C₆)-alkyl, (C₂-C₆)-alkenyl or alkynyl, or (C₃-C₆) cycloalkyl;

T is a five to eleven membered monocyclic or bicyclic, aromatic or non-aromatic ring having zero to three heteroatoms independently selected from O, N or NH, wherein T has at least one R¹⁰ substituent and up to three more substituents selected from R¹⁰ or J;

According to a preferred embodiment, W₁ in compounds of formula IE is —NH—, —CH₂—NH— or —C(O)—NH—.

Preferred embodiments of W₂ in compounds of formula IE are as recited above for R¹⁰ in compounds of formula IA.

Preferred embodiments of R, p, and T in compounds of formula IE are as recited for R, P, and T in compounds of formula IA.

According to another preferred embodiment of compounds of formula IE, p is 0 and T is selected from phenyl or naphthyl, wherein T has at least one R¹⁰ substituent and up to three more substituents selected from R¹⁰ or J. Preferably, T has three R¹⁰ substituents. More preferably, T has two R¹⁰ substituents.

Preferred compounds of formula (1E) are as shown in the Table 5, compound nos. 600-624, below.

| Cmpd# | Name |
|---|---|
| 600 | 1-Naphthalen-2-yl-3-{4-[4-(2- |
| | trifluoromethyl-phenoxymethyl)-phenyl]- |
| | piperidin-3-ylmethyl}-urea |
| 601 | Naphthalene-2-sulfonic acid {4-[4-(2- |
| | trifluoromethyl-phenoxymethyl)-phenyl]- |
| | piperidin-3-ylmethyl}-amide |
| 602 | {1-(1H-Indol-3-ylmethyl)-2-oxo-2-[2-({4- |
| | [4-(2-trifluoromethyl-phenoxymethyl)- |
| | phenyl]-piperidin-3-ylmethyl}-carbamoyl)- |
| | pyrrolidin-1-yl]-ethyl}-carbamic acid 9H- |
| | fluoren-9-ylmethyl ester |
| 603 | {4-[4-(2-Trifluoromethyl-phenoxymethyl)- |
| | phenyl]-piperidin-3-ylmethyl}-carbamic |
| | acid naphthalen-1-yl ester |
| 604 | {4-[4-(2-Trifluoromethyl-phenoxymethyl)- |
| | phenyl]-piperidin-3-ylmethyl}-carbamic |
| | acid naphthalen-2-yl ester |
| 605 | (2-Phenyl-1-{[({4-[4-(2-trifluoromethyl- |
| | phenoxymethyl)-phenyl]-piperidin-3- |
| | ylmethyl}-carbamoyl)-methyl]-carbamoyl}- |
| | ethyl)-carbamic acid benzyl ester |
| 606 | 1-Naphthalen-1-yl-3-{4-[4-(2- |
| | trifluoromethyl-phenoxymethyl)-phenyl]- |
| | piperidin-3-ylmethyl}-urea |
| 607 | {1-Benzyl-2-oxo-2-[2-({4-[4-(2- |
| | trifluoromethyl-phenoxymethyl)-phenyl]- |
| | piperidin-3-ylmethyl}-carbamoyl)- |
| | pyrrolidin-1-yl]-ethyl}-carbamic acid |
| | benzyl ester |
| 608 | Naphthalene-2-carboxylic acid {4-[4-(2- |
| | trifluoromethyl-phenoxymethyl)-phenyl]- |
| | piperidin-3-ylmethyl}-amide |
| 609 | Naphthalene-1-carboxylic acid {4-[4-(2- |
| | trifluoromethyl-phenoxymethyl)-phenyl]- |
| | piperidin-3-ylmethyl}-amide |
| 610 | 4-[4-(2-Trifluoromethyl-phenoxymethyl)- |
| | phenyl]-piperidine-3-carboxylic acid |
| | benzyl-naphthalen-2-yl-amide |
| 611 | 4-[4-(2-Trifluoromethyl-phenoxymethyl)- |
| | phenyl]-piperidine-3-carboxylic acid |
| | (1,2,3,4-tetrahydro-naphthalen-2-yl)- |
| | amide |
| 612 | 4-Biphenyl-4-yl-piperidine-3-carboxylic |
| | acid naphthalen-2-ylamide |
| 613 | 4-[4-(2-Trifluoromethyl-phenoxymethyl)- |
| | phenyl]-piperidine-3-carboxylic acid |
| | naphthalen-2-ylamide |
| 614 | 4-Biphenyl-4-yl-piperidine-3-carboxylic |
| | acid (1-naphthalen-1-yl-ethyl)-amide |
| 615 | 4-[4-(2-Trifluoromethyl-phenoxymethyl)- |
| | phenyl]-piperidine-3-carboxylic acid |
| | (naphthalen-1-ylmethyl)-amide |
| 616 | 3-({4-[4-(2-Trifluoromethyl- |
| | phenoxymethyl)-phenyl]-piperidin-3- |
| | ylmethyl}-carbamoyl)-naphthalene-2- |
| | carboxylic acid |
| 617 | 2-{4-[4-(2-Trifluoromethyl- |
| | phenoxymethyl)-phenyl]-piperidin-3- |
| | ylmethyl}-isoindole-1,3-dione |
| 618 | 4-[4-(2-Trifluoromethyl-phenoxymethyl)- |
| | phenyl]-piperidine-3-carboxylic acid |
| | benzhydryl-amide |
| 619 | 4-[4-(2-Trifluoromethyl-phenoxymethyl)- |
| | phenyl]-piperidine-3-carboxylic acid |
| | (1,2,3,4-tetrahydro-naphthalen-1-yl)- |
| | amide |
| 620 | 4-[4-(2-Trifluoromethyl-phenoxymethyl)- |
| | phenyl]-piperidine-3-carboxylic acid 2- |
| | trifluoromethoxy-benzylamide |
| 621 | 2-({4-[4-(2-Trifluoromethyl- |
| | phenoxymethyl)-phenyl]-piperidin-3- |
| | ylmethyl}-carbamoyl)- |
| | cyclohexanecarboxylic acid |
| 622 | (3,4-Dihydro-1H-isoquinolin-2-yl)-{4-[4- |
| | (2-trifluoromethyl-phenoxymethyl)- |
| | phenyl]-piperidin-3-yl}-methanone |
| 623 | 4-[4-(2-Trifluoromethyl-phenoxymethyl)- |
| | phenyl]-piperidine-3-carboxylic acid |
| | phenylamide |
| 624 | 4-[4-(2-Trifluoromethyl-phenoxymethyl)- |
| | phenyl]-piperidine-3-carboxylic acid |
| | (furan-2-ylmethyl)-amide |

According to another embodiment, the present invention provides compounds of formula II:

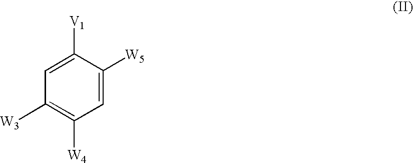

wherein:

V₁ is selected from:

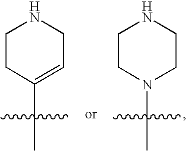

wherein V₁ is optionally substituted with R¹⁰;

W₃ is hydrogen or

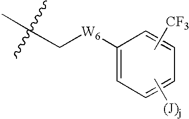

wherein:

W6 is selected from —O—, —S—, or —NH—;

j is 0 to 3;

W₄ is hydrogen or a 5-11 membered monocyclic or bicyclic aromatic ring having 0-3 heteroatoms independently selected from O, S, N, or NH, wherein W₄ has up to 3 J substituents;

W₅ is hydrogen or R¹⁰;

provided that at least two or W₃, W₄, and W₅ are simultaneously non-hydrogen;

R¹⁰ is P1-R1-P2-R2-W;

J is halogen, —R′, —OR′, —NO₂, —CN, —CF₃, —OCF₃, oxo, 1,2-methylenedioxy, —N(R′)₂, —SR′, —S(O)R′, —S(O)N(R′)₂, —SO₂R′, —C(O)R′, —CO₂R′, —C(O)N(R′)₂, —N(R′)C(O)R′, —N(R′)C(O)OR′, —N(R′)C(O)N(R′)₂, or —OC(O)N(R′)₂, wherein R′ is independently selected from hydrogen, aliphatic, heterocyclyl, heterocycly-alkyl, aryl, aralkyl, heteroaryl, or heteroaralkyl;

wherein R′ is optionally substituted with up to 3 substituents selected independently from —R¹¹, —OR¹¹, —NO₂, —CN, —CF₃, —OCF₃, oxo, 1,2-methylenedioxy, —N(R¹¹)₂, —SR¹¹, —S(O)R¹¹ , —S(O)N(R¹¹)₂, —SO₂R¹¹, —C(O)R11, —CO₂R , —C(O)N(R¹¹)₂, —N(R¹¹)C(O)R′, —N(R¹¹)C(O)OR¹¹, N(R¹¹)C(O)N(R¹¹)₂, or —OC(O)N(R¹¹)₂,;

R¹¹ is hydrogen, (C₁-C₆)-alkyl, (C₂-C₆)-alkenyl or alkynyl, or (C₃-C₆)cycloalkyl;

P1 and P2 each are independently:

absent; or

aliphatic;

R1 and R2 each are independently:

absent; or

R;

R is a suitable linker; and

W is five to eleven membered monocyclic or bicyclic, aromatic or non-aromatic ring having zero to three heteroatoms independently selected from O, S, N, or NH, wherein W has up to 3 J substituents.

According to a preferred embodiment, j is selected from 1, 2 or 3.

According to a preferred embodiment, W₃ is 2-trifluoromethyl-phenoxymethyl.

According to another preferred embodiment, V₁ is unsubstituted 3,4-didehydropiperidyl.

According to another preferred embodiment, V₁ is unsubstituted piperazyl.

According to a preferred embodiment, W or W₄ is independently phenyl or a five to seven membered monocyclic, aromatic ring having 1-3 heteroatoms independently selected from O, S, N, or NH, wherein W or W₄ has up to 3 substituents independently selected from J.

According to a more preferred embodiment, W or W₄ is selected from 2-furanyl, 3-furanyl, 3-furazanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 1-pyrrolyl, 2-pyrrolyl, 3-pyrrolyl, 1-pyrazolyl, 2-pyrazolyl, 3-pyrazolyl, 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, or 3-thienyl, wherein W or W₄ has up to 3 J substituents.

According to a preferred embodiment, W or W₄ is an eight to eleven membered bicyclic ring, wherein either or both rings is aromatic, and either or both rings has zero to three heteroatoms independently selected from O, S, N, or NH, wherein W or W₄ has up to 3 substituents independently selected from J.

According to a more preferred embodiment, W or W₄ is selected from naphthyl, 3-1H-benzimidazol-2-one, (1-substituted)-2-oxo-benzimidazol-3-yl, 1-phthalimidinyl, benzoxanyl, benzopyrrolidinyl, benzopiperidinyl, benzoxolanyl, benzothiolanyl, benzothianyl, indolinyl, chromanyl, phenanthridinyl, tetrahydroquinolinyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, indolyl, isoindolyl, acridinyl, benzoisoxazolyl, tetrahydroquinolinyl, tetrahydroisoquinolinyl, or pyrido[3,4-d]pyrimidiny, wherein W or W₄ has up to 3 J substituents.

According to another preferred embodiment W₄ is phenyl or 5-hydroxyphenyl.

According to a preferred embodiment, W₅ is P1-R1-W or R1-P2-W.

According to a more preferred embodiment, wherein each of P1 and P2 is independently (C₁-C₆)-alkyl, and R1 is R.

According to a preferred embodiment, R is selected from —CH₂—, —O—, —S—, —SO—, —SO₂—, —NR′—, —C(O)O—, —OC(O)—, —C(O)NR′—, —NR′C(O)—, —O—, —OC(O)NR′—, —NR′C(O)O—, —NR′C(O)NR′—, —NR′C(O)O—, —SO—NR′, —NR′SO—, —NR′SO₂—, —SO₂NR′—, —CHOR′—, —CHNR′—, or —C(O)—.

According to a preferred embodiment of formula (II),

each of P1 and P2 is methylene;

R1 is —O—, —NH—C(O)—, —C(O)—NH—, or —NH—; and

W is selected from phenyl, 4-hydroxyphenyl, 1-napthyl, 2-napthyl, isoquinolinyl, quinolinyl, or 2-trifluoromethylphenyl.

According to a more preferred embodiment, J is independently selected from halogen, —R′, —OR′, —NO₂, —CN, —CF₃, —OCF₃, oxo, 1,2-methylenedioxy, —N(R′)₂, —SR′, —S(O)R′, —S(O)N(R′)₂, —SO₂R′, —C(O)R′, —CO₂R′ or —C(O)N(R′)₂, wherein R′ is independently selected from hydrogen or (C₁-C₆)-alkyl.

According to a preferred embodiment, wherein in W₃, j is 1-3.

The compounds utilized in this invention are limited to those that are chemically feasible and stable. Therefore, a combination of substituents or variables in the compounds described above is permissible only if such a combination results in a stable or chemically feasible compound. A stable compound or chemically feasible compound is one in which the chemical structure is not substantially altered when kept at a temperature of 40° C. or less, in the absence of moisture or other chemically reactive conditions, for at least a week.

The BACE inhibitors of this invention may contain one or more “asymmetric” carbon atoms and thus may occur as racemates and racemic mixtures, single enantiomers, diastereomeric mixtures and individual diastereomers. All such isomeric forms of these compounds are expressly included in the present invention. Each stereogenic carbon may be of the R or S configuration. Although specific compounds and scaffolds exemplified in this application may be depicted in a particular stereochemical configuration, compounds and scaffolds having either the opposite stereochemistry at any given chiral center or mixtures thereof are also envisioned.

Unless otherwise stated, structures depicted herein are also meant to include compounds that differ only in the presence of one or more isotopically enriched atoms. For example, compounds having the present structures except for the replacement of a hydrogen by a deuterium or tritium, or the replacement of a carbon by a ¹³C— or ¹⁴C-enriched carbon are within the scope of this invention.

The compounds of this invention may be prepared as illustrated by the Schemes I-VIII below and by general methods known to those skilled in the art.

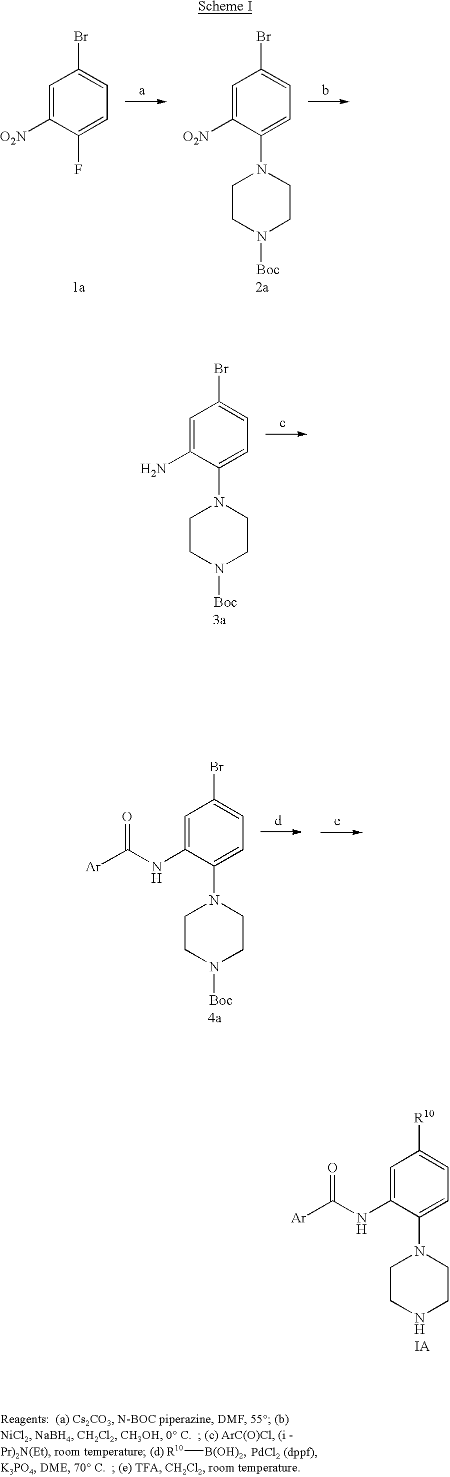

Scheme I above shows a general route for the preparation of compounds of formula IA. Displacement of commercially available 4-bromo-1-fluoro-2-nitrobenzene 1a with commercially available piperazine-1-carboxylic acid tert-butyl ester provided intermediate 2a. Nitro reduction of intermediate 2a followed by acylation with a suitable acyl chloride provided intermediate 4a. Substituent R¹⁰ was then introduced using a boronic acid under palladium catalysis followed by trifluoroacetic acid mediated cleavage of the BOC protecting group to give compounds of formula IA.

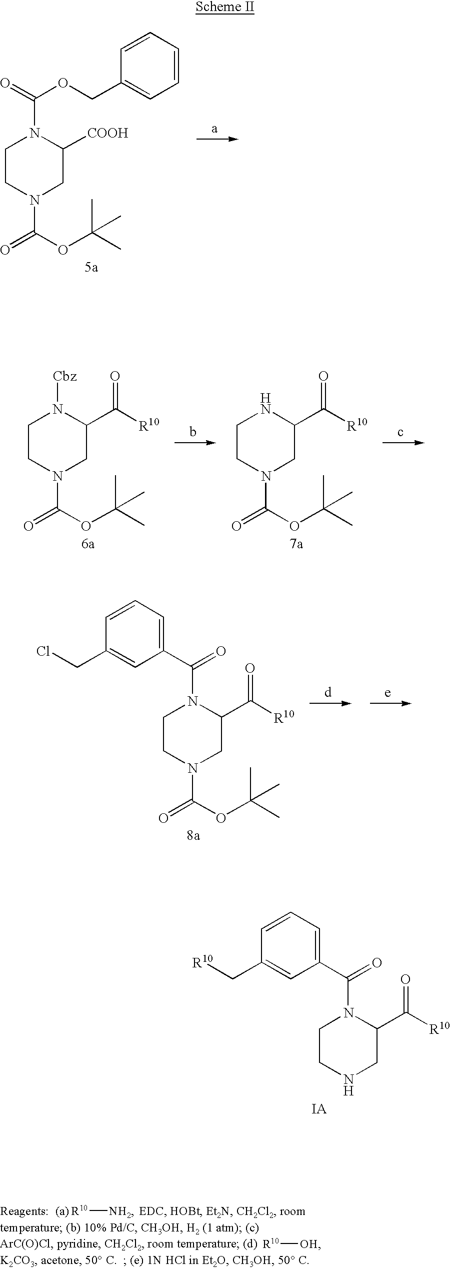

Scheme II above shows another general route for the preparation of compounds of formula IA. Commercially available acid 5a was converted to amide intermediate 6a. Hydrogenolysis of the Cbz protecting group followed by acylation provided intermediate 8a. Displacement of the benzyl chloride in 8a with R¹⁰ phenol followed by ethereal HCl mediated removal of the BOC protecting group afforded compounds of formula IA.

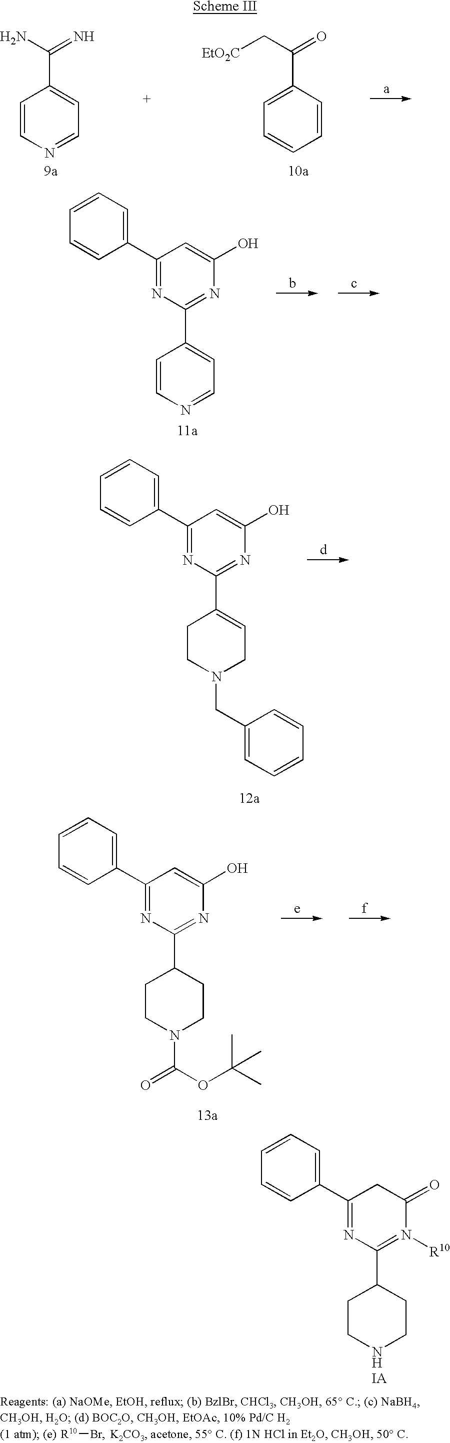

Scheme III above shows another general route for the preparation of compounds of formula IA. Commercially available amidino pyridine 9a was cyclo-condensed with commercially available ethyl ester 10a to provide pyrimidine intermediate 11a. Alkylation and subsequent reduction provided 12a. Reduction and benzyl deprotection with in situ reprotection with BOC anhydride afforded intermediate 13a. Alkylation with a suitable R¹⁰ benzyl halide followed by ethereal HCl mediated removal of the BOC protecting group afforded compounds of formula IA.

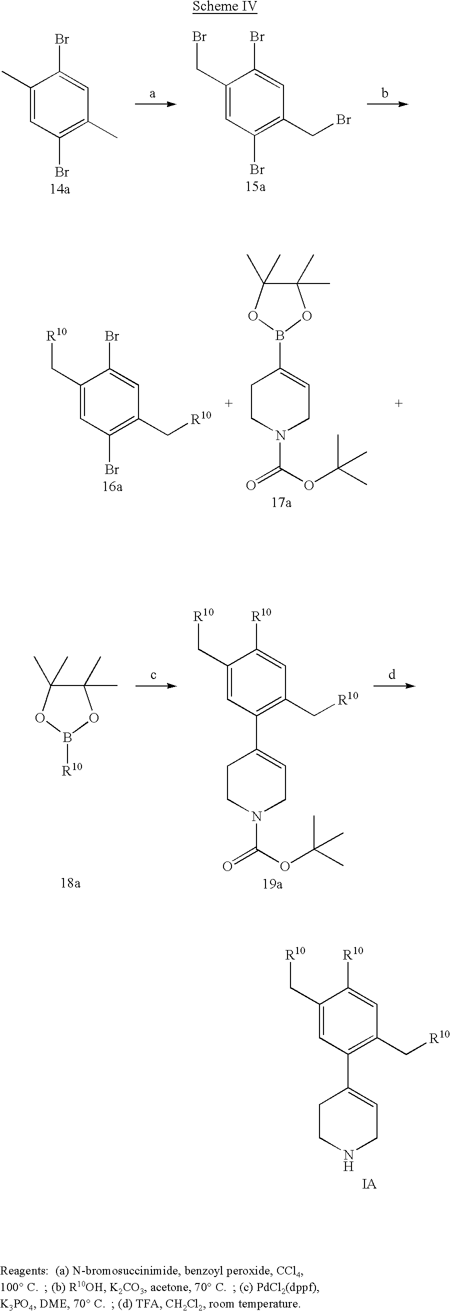

Scheme IV above shows another general route for the preparation of compounds of formula IA. Commercially available dibromoxylene 14a was converted to tetrabromide 15a and further displaced with R¹⁰ phenols to give intermediate dibromide 16a. A Suzuki type coupling with cyclic boronates 17a and 18a yielded intermediate 19a. Boronate 17a was prepared according to the method reported in Tetrahedron Letters, 41(19), 3705-3708 (2000). Final trifluoroacetic acid mediated cleavage of the BOC protecting group gave compounds of formula IA.

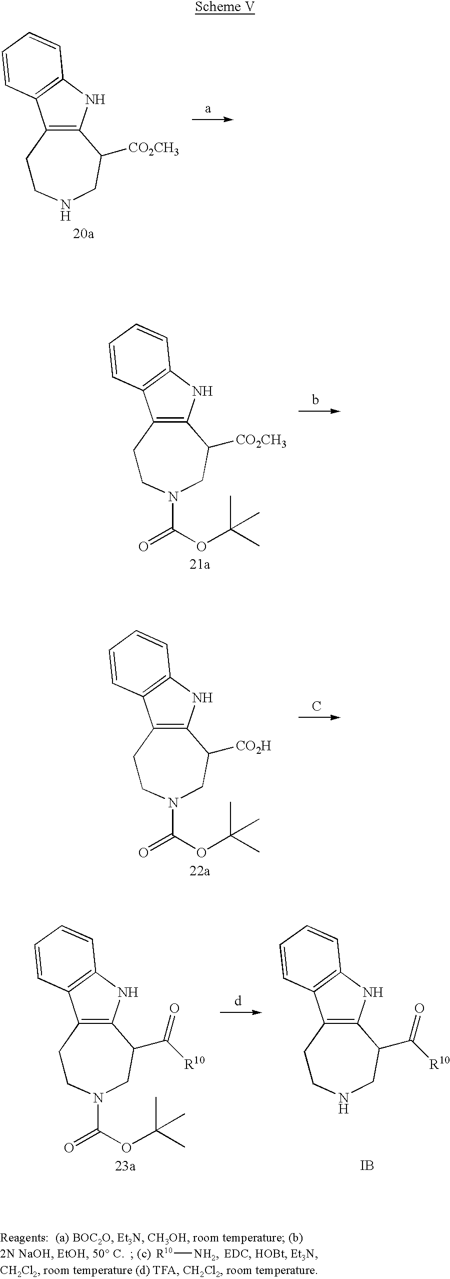

Scheme V above shows a general route for the preparation of compounds of formula IB. Commercially available azepine ester 20a was N-protected followed by ester hydrolysis to give intermediate acid 22a. Coupling with a suitable R¹⁰-amine followed by trifluoroacetic acid mediated deprotection provided compounds of formula IB.

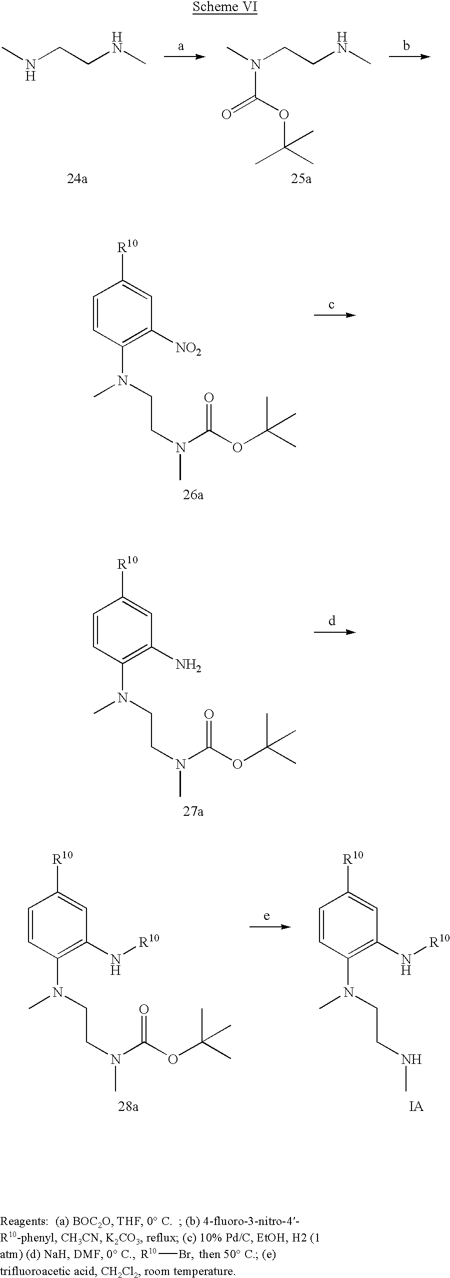

Scheme VI above shows another general route for the preparation of compounds of formula IA. Commercially available diamine 24a was N-protected then used to displace a commercially available aryl flouride to give intermediate 26a. Palladium mediated nitro reduction gave intermediate 27a which was then alkylated with a suitable R¹⁰ bromide to afford intermediate 28a. N-BOC deprotection with trifluoroacetic acid gave compounds of formula IA.

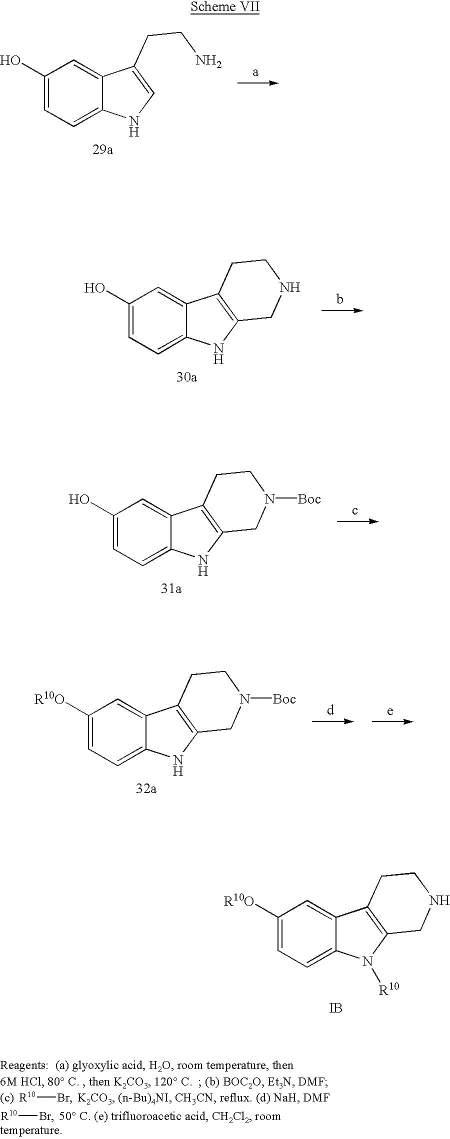

Scheme VII above shows a general route for the preparation of compounds of formula IB. Commercially available 5-hydroxytryptamine 29a was converted to intermediate carboline 30a. Further N-protection with Boc anhydride gives compound 31a. Etherification with a suitable R¹⁰-bromide, followed by N alkylation with another R¹⁰-bromide and final N-Boc removal with trifluoroacetic acid gave compounds of formula Ib.

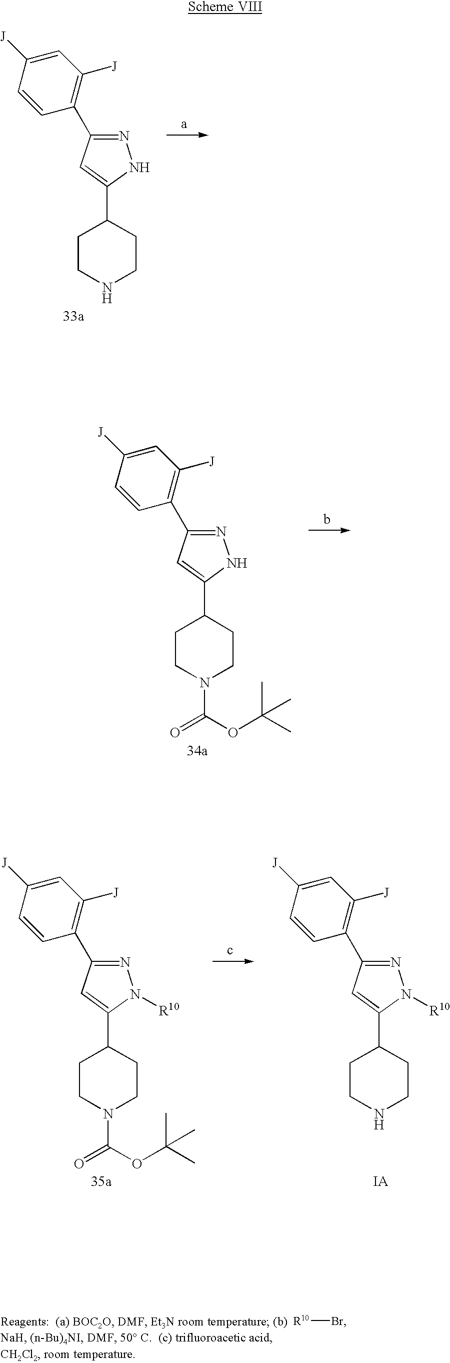

Scheme VIII above shows a general route for the preparation of compounds of formula IA. Commercially available pyrazole 33a was N-protected with Boc anhydride to provide intermediate 34a. Pyrazole alkylation followed by deprotection of the N-Boc group with trifluoroacetic acid provided compounds of formula IA.

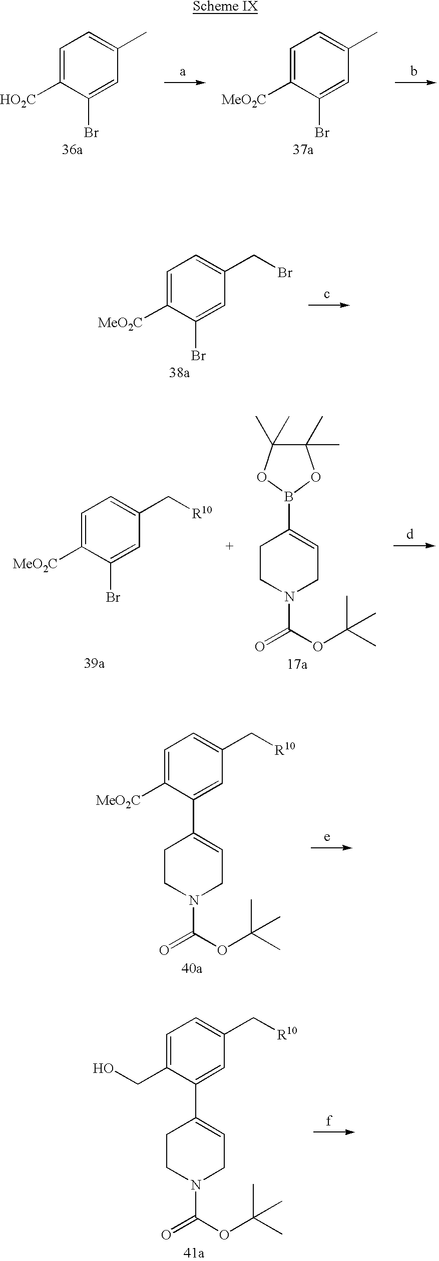

Scheme IX above shows another general route for the preparation of compounds of formula IA. Commercially available benzoic acid 36a was esterified then converted to benzyl bromide 38a. Displacement with a suitable R¹⁰-OH followed by Suzuki coupling gave intermediate ester 40a. Reduction of the ester and conversion to the chloride yielded compound 42a Subsequent displacement of the chloride followed by N-Boc deprotection gave compounds of formula IA.

One having ordinary skill in the art may synthesize other compounds of this invention following the teachings of the specification using reagents that are readily synthesized or commercially available.

According to another embodiment, the present invention provides a composition for inhibit BACE activity in a mammal, comprising compounds of formula IA, formula IB, formula ICa, formula ICb, formula ID or formula IE or a pharmaceutically acceptable salt thereof and a pharmaceutically acceptable carrier, adjuvant, or vehicle. The amount of compound in the compositions of this invention is such that it is effective to detectably inhibit an aspartic proteinase, particularly BACE in a biological sample or in a patient. Preferably the composition of this invention is formulated for administration to a patient in need of such composition. Most preferably, the composition of this invention is formulated for oral administration to a patient.

In another embodiment, the pharmaceutical composition of the present invention is comprised of a compound of formula IA, formula IB, formula ICa, formula ICb, formula ID, or formula IE, a pharmaceutically acceptable carrier, and a neurotrophic factor.

The term “neurotrophic factor,” as used herein, refers to compounds which are capable of stimulating growth or proliferation of nervous tissue. Numerous neurotrophic factors have been identified in the art and any of those factors may be utilized in the compositions of this invention. These neurotrophic factors include, but are not limited to, nerve growth factor (NGF), insulin-like growth factor (IGF-1) and its active truncated derivatives such as gIGF-1 and Des(1-3)IGF-I, acidic and basic fibroblast growth factor (aFGF and bFGF, respectively), platelet-derived growth factors (PDGF), brain-derived neurotrophic factor (BDNF), ciliary neurotrophic factors (CNTF), glial cell line-derived neurotrophic factor (GDNF), neurotrophin-3 (NT-3)and neurotrophin 4/5 (NT-4/5). The most preferred neurotrophic factor in the compositions of this invention is NGF.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The term “detectably inhibit”, as used herein means a measurable change in BACE activity between a sample comprising said composition and a BACE proteinase and an equivalent sample comprising BACE proteinase in the absence of said composition.

A “pharmaceutically acceptable salt” means any non-toxic salt, ester, salt of an ester or other derivative of a compound of this invention that, upon administration to a recipient, is capable of providing, either directly or indirectly, a compound of this invention or an inhibitorily active metabolite or residue thereof.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N⁺ (C₁₋₄ alkyl)₄ salts. This invention also envisions the quaternization of any basic nitrogen-containing groups of the compounds disclosed herein. Water or oil-soluble or dispersible products may be obtained by such quaternization.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, the pharmaceutically acceptable compositions of this invention are formulated for oral administration.

The amount of the compounds of the present invention that may be combined with the carrier materials to produce a composition in a single dosage form will vary depending upon the host treated, the particular mode of administration. Preferably, the compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Depending upon the particular condition, or disease, to be treated or prevented, additional therapeutic agents, which are normally administered to treat or prevent that condition, may also be present in the compositions of this invention.

Examples of agents the compounds of this invention may also be combined with include, without limitation, anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents for treating diabetes such as insulin, insulin analogues, alpha glucosidase inhibitors, biguamides, and insulin sensitizers; and agents for treating immunodeficiency disorders such as gamma globulin.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

According to another embodiment, the invention relates to a method of inhibiting BACE activity in a biological sample comprising the step of contacting said biological sample with a compound of this invention, or composition comprising said compound. According to a preferred embodiment, the invention relates to a method of inhibiting BACE proteinase activity in a biological sample comprising the step of contacting said biological sample with a compound of formula IA, formula IB, formula ICa, formula ICb, formula ID or formula IE.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of BACE activity in a biological sample is useful for a variety of purposes which are known to one of skill in the art. Examples of such purposes include, but are not limited to, blood transfusion, organ-transplantation, biological specimen storage, and biological assays.

According to another embodiment, the invention provides a method for treating or lessening the severity of a BACE-mediated disease or condition in a patient comprising the step of administering to said patient a composition according to the present invention.

The term “BACE-mediated disease”, as used herein, means any disease or other deleterious condition or disease in which BACE is known to play a role. Such a disease or condition includes Alzheimer's Disease, MCI (“mild cognitive impairment”), Down's syndrome, hereditary cerebral hemorrhage, cerebral amyloid angiopathy, dementia, including dementia of mixed vascular and degenerative origin, dementia associated with Parkinson's disease, progressive supranuclear palsy or cortical basal degeneration.

In an alternate embodiment, the methods of this invention that utilize compositions that do not contain an additional therapeutic agent, comprise the additional step of separately administering to said patient an additional therapeutic agent. When these additional therapeutic agents are administered separately they may be administered to the patient prior to, sequentially with or following administration of the compositions of this invention.

The compounds of this invention or pharmaceutical compositions thereof may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition. Implantable devices coated with a compound of this invention are another embodiment of the present invention.

In order that the invention described herein may be more fully understood, the following examples are set forth. It should be understood that these examples are for illustrative purposes only and are not to be construed as limiting this invention in any manner.

### EXAMPLES

General Methods:

Method A.

The piperidine N-boc cleavage by TFA: The starting material (normally 10 to 30 mg) was dissolved in 20% trifluoroacetic acid in dichloromethane (3 ml). After stirring at RT for 40 min, the reaction was evaporated to dryness to give the TFA salt of the substituted piperidines.

Method B.

The piperidine N-boc cleavage by HCl: The starting material (10 to 30 mg) was dissolved or suspended in methanol (3 ml) and a solution of 1 N HC1-ether (3 ml) was added. After stirring at 50C for 40 min, the reaction was evaporated to dryness to give the HCl salt of the substituted piperidines.

HPLC.

For analytical HPLC, a HP series 1100 system was used, with a 3.0×150 mm YMC ODS-AQ 5.5 μ 120 Å column, the solvents system were run according to the following order:

| Time (min) | H₂O (%) | CH₃CN (%) |
|---|---|---|
| 0 | 90 | 10 |
| 1 | 90 | 10 |
| 8 | 10 | 90 |
| 10 | 10 | 90 |
| 11 | 90 | 10 |
| 12 | 90 | 10 |

### Example 1

Preparation of Compound 108

4-(4-Bromo-2-nitro-phenyl)-piperazine-1-carboxylic Acid tert-butyl Ester (1B):

4-Bromo-1-fluoro-2-nitro-benzene (5.0 g, 22.7 mmol) was dissolved in 30 mL DMF with piperazine-1-carboxylic acid tert-butyl ester (5.0 g, 26.9 mmol) and cesium carbonate (10.0 g, 30.8 mmol) and heated to 55° C. for 10 hours, then let stir at room temperature for 6 more hours The reaction mixture was diluted with ethyl acetate and the organic layer washed with 10% citric acid, saturated sodium bicarbonate and brine and then dried over magnesium sulfate, filtered and concentrated to give 4-(4-bromo-2-nitro-phenyl)-piperazine-1-carboxylic acid tert-butyl ester as an orange oil, 8.7 g, 22.7 mmol, 100% yield. ¹H NMR (500 MHz, CDCl₃) 7.72 ppm (1H, s), 7.34 ppm (1H, d), 6.78 ppm (1H, d), 3.32 ppm (4H, m), 2.79 ppm (4H, m), 1.25 ppm (9H, s)

4-(2-Amino-4-bromo-phenyl)-piperazine-1-carboxylic Acid tert-butyl Ester (2B):

4-(4-Bromo-2-nitro-phenyl)-piperazine-1-carboxylic acid tert-butyl ester, (9.2 g, 23.8 mmol) was dissolved in a 1:1 mixture of methylene chloride and methanol and cooled to 0° C. To this solution was added NiCl₂ hexahydrate (0.24 g, 1 mmol) followed by NaBH₄ (1.5 g, 39.5 mmol) in portions over one hour. The reaction mixture went from orange to colorless and then to black. The solvent was removed under reduced pressure and the residue was applied to a silica column with methylene chloride and eluted with 20% ethyl acetate in hexanes to give 4-(2-amino-4-bromo-phenyl)-piperazine-1-carboxylic acid tert-butyl ester as a white foam, 7.6 g, 21.4 mmol, 96% yield. ¹H NMR (500 MHz, CDCl₃) 6.80 ppm (1H, s), 6.75 ppm (2H, m), 3.50 ppm (4H, br s), 2.75 ppm (4H, br, s), 1.41 ppm (9H, s).

4-{4-Bromo-2-[(naphthalene-1-carbonyl)-amino]-phenyl}-piperazine-1-carboxylic Acid tert-butyl Ester (3B):

4-(2-Amino-4-bromo-phenyl)-piperazine-1-carboxylic acid tert-butyl ester (2.6 g, 7.3 mmol) was dissolved in methylene chloride with DIEA (1.7 mL, 10 mmol). To this solution, 1-naphthoyl chloride (1.45 g, 7.7 mmol) was added as a neat liquid. The reaction mixture was stirred for 2 hours at room temperature, diluted with ethyl acetate and the organic layer washed with 10% citric acid, saturated sodium bicarbonate and brine and then dried over magnesium sulfate, filtered and concentrated to a brown oil which was purified by silica chromatography (15% ethyl acetate/hexanes) to give 4-{4-bromo-2-[(naphthalene-1-carbonyl)-amino]-phenyl}-piperazine-1-carboxylic acid tert-butyl ester, 3.4 g, 6.7 mmol, 91% yield. ¹H NMR (500 MHz, CDCl₃) 9.12 ppm (1H, s), 8.83 ppm (1H, s), 8.35 ppm (1H, d), 7.94 ppm (1H, d), 7.85 ppm, (1H, d), 7.69 ppm (1H, d), 7.50 ppm (3H, m), 7.2 ppm (1H, d), 6.99 ppm (1H, d), 3.40 ppm (4H, br s), 2.75 ppm (4H, br s), 1.40 ppm (9H, s).

Naphthalene-1-carboxylic acid (2′,5′-dichloro-4-piperazin-1-yl-biphenyl-3-yl)-amide (Compound 108):

4-{4-Bromo-2-[(naphthalene-1-carbonyl)-amino]-phenyl}-piperazine-1-carboxylic acid tert-butyl ester (50 mg, 0.1 mmol) was placed in a screw cap test tube and dissolved in 4 ml of DME with potassium phosphate (80 mg, 0.38 mmol), and 2,5-dichlorophenyl boronic acid (50 mg, 0.26 mmol). To this mixture was added Pd(dppf)Cl₂ (10 mg, mmol), argon was bubbled through for 1 min, and the reaction sealed and heated to 70° C. for 16 hours. The reaction mixture was diluted with ethyl acetate, filtered, and the filtrate concentrated to an oil which was purified by silica chromatography (15% ethyl acetate/hexanes eluent) to give the t-boc protected product MS MH+576.0. This material was dissolved in 1 mL methylene chloride and 1 mL TFA was added and the reaction mixture let stand for 1 hr. The solvent was then removed and the residue crystallized from methanol/Et₂O to give naphthalene-1-carboxylic acid (2′,5′-dichloro-4-piperazin-1-yl-biphenyl-3-yl)-amide as a TFA salt, 30 mg, 0.051 mmol, 51% yield. LC/ms ret. time 2.86 min. MH+476.0. ¹H NMR (500 MHz, CD₃OD) 8.33 ppm (1H, d), 8.31 ppm (1H, m), 8.06 ppm (1H, d), 7.98 ppm (1H, d), 7.83 ppm (1H, m), 7.61 ppm (3H, m), 7.55 ppm (1H, d), 7.52 ppm (1H, m)7.38 ppm (3H, m), 3.3 ppm (8H, m).

### Example 2

Preparation of Compound 166

4-{4-Bromo-2-[(naphthalen-2-ylmethyl)-amino]-phenyl}-piperazine-1-carboxylic Acid tert-butyl Ester (4B):

4-(2-Amino-4-bromo-phenyl)-piperazine-1-carboxylic acid tert-butyl ester (0.20 g, 0.56 mmol) was dissolved in DMF with 2-naphthylmethyl bromide (0.12 g, 0.56 mmol). To this solution sodium hydride (24 mg, 1 mmol) was added. The reaction mixture was stirred overnight, diluted with ethyl acetate, and the organic layer was washed with brine, dried over magnesium sulfate, filtered and concentrated to an oil. This oil was purified by silica chromatography to give 4-{4-bromo-2-[(naphthalen-2-ylmethyl)-amino]-phenyl}-piperazine-1-carboxylic acid tert-butyl ester, 80 mg, 0.16 mmol, 29% yield. ¹H NMR (500 MHz, CDCl₃) 7.73 ppm (4H, m), 7.32 ppm (3H, m), 6.75 ppm (3H, m), 4.40 ppm (2H, s), 1.41 ppm (9H, s).

4-{3-[(Naphthalen-2-ylmethyl)-amino]-4′-trifluoromethyl-biphenyl-4-yl}-piperazine-1-carboxylic Acid tert-butyl Ester (5B):

4-{4-Bromo-2-[(naphthalen-2-ylmethyl)-amino]-phenyl}-piperazine-1-carboxylic acid tert-butyl ester (40 mg, 0.08 mmol) was placed in a screw cap test tube and dissolved in 4 ml of DME with potassium phosphate (80 mg, 0.38 mmol), and 4-trifluoromethylphenyl boronic acid (50 mg, 0.26 mmol). To this mixture was added Pd(dppf)Cl₂ (10 mg, 0.014 mmol), argon was bubbled through for 1 min, and the reaction sealed and heated to 70° C. for 16 hours. The reaction mixture was diluted with ethyl acetate, filtered, and the filtrate concentrated to an oil which was purified by silica chromatography (10% ethyl acetate/hexane eluent) to give 4-{3-[(naphthalen-2-ylmethyl)-amino]-4′-trifluoromethyl-biphenyl-4-yl}-piperazine-1-carboxylic acid tert-butyl ester, 30 mg, 0.05 mmol, 67% yield, ms MH+562.3.

Naphthalen-2-ylmethyl-(4-piperazin-1-yl-4′-trifluoromethyl-biphenyl-3-yl)-amine (Compound 166):

4-{3-[(Naphthalen-2-ylmethyl)-amino]-4′-trifluoromethyl-biphenyl-4-yl}-piperazine-1-carboxylic acid tert-butyl ester (30 mg. 0.05 mmol) was dissolved in 1 mL methylene chloride and 1 mL TFA added. After one hour, the solvent was removed, the residue was treated with Et₂O and a white solid, naphthalen-2-ylmethyl-(4-piperazin-1-yl-4′-trifluoromethyl-biphenyl-3-yl)-amine was filtered off, 13 mg, 0.023 mmol, 46% yield, ms MH+462.2, ¹H NMR (500 MHz, CD₃OD) 7.95 (4H, m), 7.57 (5H, m), 7.42 (2H, m) 7.12 (1H, d), 6.94 (1H, d) 6.87 (1H, s), 4.73 (2H, s), 3.43 (4H, m), 3.20 (4H, br s).

### Example 3

Preparation of Compound 168

4-5(Chloro-2-nitro-4-trifluoromethyl-phenyl)-piperazine-1-carboxylic Acid tert-butyl Ester (6B):

1,5 Dichloro-2-nitro-4-triflouromethyl-benzene (1.50 g, 5.76 mmol) was dissolved in 20 ml DMF with TEA (0.87 g, 8.64 mmol) and piperazine-1-carboxylic acid tert-butyl ester (1.06 g, 5.76 mmol) and heated to 60° C. for three hours. The reaction mixture was cooled to room temperature and diluted with a 80% mixture of ethyl acetate in hexane, and the organic layer was washed with water, brine and then dried over magnesium sulfate, filtered and concentrated under reduced pressure. The residue was applied to a silica column with methylene chloride and eluted with 20% ethyl acetate in hexane to give 4-5(chloro-2-nitro-4-trifluoromethyl-phenyl)-piperazine-1-carboxylic acid tert-butyl ester as a yellow solid, 1.8 g, 4.39 mmol, 76%. ¹H NMR (500 MHz, CDCl₃) 8.18 ppm (1H, s), 7.15 ppm (1H, s), 3.62 ppm (4H, m), 3.15 ppm (4H, m), 1.48 ppm (9H, s).

4-(3′, 4′-Dichloro-4-nitro-6-trifluoromethyl-biphenyl-3-yl)-piperazine-1-carboxylic Acid tert-butyl Ester (7B):

4-(5-Chloro-2-nitro-4-trifluoromethyl-phenyl)-piperazine-1-carboxylic acid tert-butyl ester, (0.1 g, 0.24 mmol) was dissolved in 5 ml of DME and purged with nitrogen for five minutes. To this solution was added potassium phosphate (0.16 g, 0.75 mmol) followed by dichloro(1,1-bis (diphenylphosphine)ferrocene) palladium (II) dichloromethane adduct (0.03 g, 0.04 mmol) and the mixture heated at 80° C. for seventy-two hours. The reaction mixture went from orange to black. After seventy-two hours the reaction was cooled to room temperature and diluted with ethyl acetate, the organics were separated and washed with saturated sodium bicarbonate, water, brine and then dried over magnesium sulfate, filtered and concentrated under reduced pressure to give a brown oil. This was taken up in 5.0 ml 0.1% TFA acetonitrile and filtered, and the filtrate was purified by HPLC (with a gradient 50-100% acetonitrile/water) to give 0.1 g (0.2 mmol, 83%) of 4-(3′, 4′-dichloro-4-nitro-6-trifluoromethyl-biphenyl-3-yl)-piperazine-1-carboxylic acid tert-butyl ester as a yellow solid. ¹H NMR (500 MHz, CDCl₃) 8.22 ppm (1H, s), 7.51 ppm (1H, m), 7.40 ppm (1H, s), 7.16 ppm (1H, m), 4.1 ppm (2H, m), 3.62 ppm (4H, m), 3.16 ppm (4H, m), 1.48 ppm (9H, s).

4-(4-Amino-3′,4′-dichloro-6-trifluoromethyl-biphenyl-3-yl)-piperazine-1-carboxylic Acid tert-butyl Ester (8B):

4-(3′, 4′-Dichloro-4-nitro-6-trifluoromethyl-biphenyl-3-yl)-piperazine-1-carboxylic acid tert-butyl ester, 0.1 g, was dissolved in methanol and degassed with nitrogen, treated with palladium, 10 wt. % on activated carbon (0.03 g) and subjected to a hydrogen atmosphere for two hours. After two hours the hydrogen was purged with nitrogen and the mixture was filtered. The resulting filtrate was evaporated and dried under high vacuum to give 4-(4-amino-3′,4′-dichloro-6-trifluoromethyl-biphenyl-3-yl)-piperazine-1-carboxylic acid tert-butyl ester as a clear oil, 0.1 g, 0.2 mmol. 4-{3′,4′-Dichloro-4-[(naphthalene-1-carbonyl)-amino]-6-trifluoromethyl-biphenyl-3-yl}-piperazine-1-carboxylic acid tert-butyl ester (9B): 4-(4-Amino-3′,4′-dichloro-6-trifluoromethyl-biphenyl-3-yl)-piperazine-1-carboxylic acid tert-butyl ester (0.1 g, 0.2 mmol) was dissolved in 5 ml of methylene chloride and to this solution was added TEA (0.03 g, 0.3 mmol) and 2 equivalents of 1-naphthoyl chloride (0.08 g, 0.4 mmol). The resulting solution was stirred at room temperature for eighteen hours, evaporated to dryness, taken up in 5.0 ml 0.1% TFA acetonitrile and filtered. The filtrate was then purified by HPLC (with a gradient 50-100% acetonitrile/water) to give 4-{3′,4′-dichloro-4-[(naphthalene-1-carbonyl)-amino]-6-trifluoromethyl-biphenyl-3-yl }-piperazine-1-carboxylic acid tert-butyl ester as a white solid 0.037 g, 0.06 mmol 24% for two steps. ¹H NMR (500 MHz, CDCl₃) 9.13 ppm (1H, s), 8.94 ppm (1H, s), 8.45 ppm (1H, m), 8.02 ppm (1H, m), 7.94 ppm (1H, m), 7.74 ppm (1H, m), 7.55 ppm (3H, m), 7.47 ppm (1H, m), 7.41 ppm (1H, m), 7.16 ppm (1H, m), 7.03 ppm (1H, s), 3.46 ppm (4H, m), 2.88 ppm (4H, m), 1.43 ppm (9H, s).

Naphthalene-1-carboxylic Acid (3′,4′-dichloro-5-piperazin-1-yl-2-trifluoromethyl-biphenyl-4-yl)-amide (Compound 168):

4-{3′,4′-Dichloro-4-[(naphthalene-1-carbonyl)-amino]-6-trifluoromethyl-biphenyl-3-yl}-piperazine-1-carboxylic acid tert-butyl ester 0.037 g, 0.06 mmol was dissolved in a 20% mixture of TFA in methylene chloride solution and stirred at room temperature for thirty minutes. After thirty minutes the solution was diluted with ethyl ether, the resulting crystals were collected and washed with cold ethyl ether then dried under reduced pressure to give naphthalene-1-carboxylic acid (3′,4′-dichloro-5-piperazin-1-yl-2-trifluoromethyl-biphenyl-4-yl)-amide as the TFA salt, 0.025 g, 0.05 mmol 79%. ¹H NMR (500 MHz, CD₃CN) 8.97 ppm (1H, s), 8.37 ppm (1H, s), 8.10 ppm (1H, m), 8.03 ppm (1H, m), 7.83 ppm (1H, m), 7.60 ppm (5H, m), 7.33 ppm (1H, m), 7.24 ppm (1H, s), 3.25 ppm (4H, m), 3.2 ppm (4H, m).

### Example 4

Preparation of Compound 171

4-(2-Nitro-4-trifluoromethyl-phenyl)-piperazine-1-carboxylic Acid tert-butyl Ester (10B):

1-(2-Nitro-4-trifluoromethyl-phenyl)-piperazine (5.0 g, 18.18 mmol) was dissolved in 50 ml 50% acetone in water at 0° C. To this solution was added sodium bicarbonate and di-tert-butyl dicarbonate (4.36 g, 20.0 mmol). The reaction mixture stirred for three hours filtered and the organic layer was removed under reduced pressure. The aqueous layer was diluted with ethyl ether. The organic layer was washed with brine and then dried over magnesium sulfate, filtered and concentrated under reduced pressure. The residue was applied to a silica column with methylene chloride and eluted with 15% ethyl acetate in hexane to give 4-(2-nitro-4-trifluoromethyl-phenyl)-piperazine-1-carboxylic acid tert-butyl ester as a yellow solid, 6.27 g, 16.7 mmol, 92%. ¹H NMR (500 MHz, CDCl₃) 8.05 ppm (1H, s), 7.59 ppm (1H, m), 6.93 ppm (1H, m), 3.62 ppm (4H, m), 3.15 ppm (4H, m), 1.48 ppm (9H, s).

4-(3′, 4′-Dichloro-4-nitro-6-trifluoromethyl-biphenyl-3-yl)-piperazine-1-carboxylic Acid tert-butyl Ester (11B):

4-(2-Nitro-4-trifluoromethyl-phenyl)-piperazine-1-carboxylic acid tert-butyl ester (6.27 g 16.7 mmol) was dissolved in 150 ml methanol, purged with nitrogen, treated with palladium, 10 wt. % on activated carbon (0.60 g) and then subjected to a hydrogen atmosphere for three hours. The reaction mixture was purged with nitrogen, filtered and concentrated under high vacuum to give 4-(3′, 4′-dichloro-4-nitro-6-trifluoromethyl-biphenyl-3-yl)-piperazine-1-carboxylic acid tert-butyl ester as a yellow solid (5.70 g, 16.50 mmol). ¹H NMR (500 MHz, CDCl₃) 7.82 ppm (1H, s), 7.53 ppm (1H, m), 7.15 ppm (1H, m), 4.75 ppm (2H, m) 3.59 ppm (4H, m), 3.05 ppm (4H, m), 1.48 ppm (9H, s).

4-[2-(4-Iodo-benzenesulfonylamino)-4-trifluoromethyl-phenyl]-piperazine-1-carboxylic Acid tert-butyl Ester (12B):

4-(3′, 4′-Dichloro-4-nitro-6-trifluoromethyl-biphenyl-3-yl)-piperazine-1-carboxylic acid tert-butyl ester, 0.35 g, 1.01 mmol, and 4-iodo-benzenesulfonyl chloride, 0.61 g, 2.00 mmol, were dissolved in 5 ml pyridine and heated to 60° C. for four hours. The reaction was cooled to room temperature, diluted with ethyl acetate and the organic layer was washed with HCl (0.5 N), saturated sodium bicarbonate, water, brine and then dried over magnesium sulfate, filtered and concentrated under reduced pressure. The residue was applied to a silica column with methylene chloride and eluted with 15% ethyl acetate in hexane to give 4-[2-(4-iodo-benzenesulfonylamino)-4-trifluoromethyl-phenyl]-piperazine-1-carboxylic acid tert-butyl ester as a yellow solid, 0.43 g, 0.70 mmol, 69%. ¹H NMR (500 MHz, CDCl₃) 7.85 ppm (3H, m), 7.52 ppm (2H, m), 7.32 ppm (1H, m), 7.17 ppm (1H, m), 3.55 ppm (4H, m), 2.57 ppm (4H, m), 1.48 ppm (9H, s).

4-[4-Trifluoromethyl-2-(4′-trifluoromethyl-biphenyl-4-sulfonylamino)-phenyl]-piperazine-1-carboxylic Acid tert-butyl Ester (13B):

4-[2-(4-Iodo-benzenesulfonylamino)-4-trifluoromethyl-phenyl]-piperazine-1-carboxylic acid tert-butyl ester, 0.1 g, 0.16 mmol, was dissolved in 4 ml of DME and purged with nitrogen for five minutes. To this solution was added potassium phosphate (0.10 g, 0.49 mmol) followed by dichloro(1,1-bis(diphenylphosphine)ferrocene) palladium (II) dichloromethane adduct (0.03 g, 0.04 mmol) and heated to 80° C. for eighteen hours. The reaction mixture went from orange to black. The reaction was cooled to room temperature, diluted with ethyl acetate and the organic layer was washed with saturated sodium bicarbonate, water, brine and then dried over magnesium sulfate. Filtration and concentration under reduced pressure gave a brown oil which was taken up in 5.0 ml 0.1% TFA acetonitrile and filtered. The filtrate was then purified by HPLC (with a gradient 50-100% acetonitrile/water) to give 4-[4-trifluoromethyl-2-(4′-trifluoromethyl-biphenyl-4-sulfonylamino)-phenyl]-piperazine-1-carboxylic acid tert-butyl ester as a yellow solid 0.04 g, 0.06 mmol, 36%. ¹H NMR (500 MHz, CDCl₃) 7.95 ppm (1H, s), 7.51 ppm (1H, m), 7.80 ppm (2H, m), 7.75 ppm (1H, m), 7.60 ppm (1H, m), 7.55 ppm (1H, m), 7.41 ppm (1H, m), 7.36 ppm (1H, m), 7.25 ppm (1H, m), 7.15 ppm (1H, m), 3.45 ppm (4H, m), 2.68 ppm (4H, m), 1.42 ppm (9H, s).

4′-Trifluoromethyl-biphenyl-4-sulfonic acid (2-piperazin-1-yl-5-trifluoromethyl-phenyl)-amide (Compound 171):

4-[4-Trifluoromethyl-2-(4′-trifluoromethyl-biphenyl-4-sulfonylamino)-phenyl]-piperazine-1-carboxylic acid tert-butyl ester, 0.04 g, 0.06 mmol, was dissolved in a solution of 20% TFA in methylene chloride and stirred at room temperature for thirty minutes. The solution was diluted with ethyl ether the resulting crystals were collected and washed with cold ethyl ether then dried under reduced pressure to give 4′-trifluoromethyl-biphenyl-4-sulfonic acid (2-piperazin-1-yl-5-trifluoromethyl-phenyl)-amide, 0.020 g, 0.05 mmol 64% as the TFA salt. ¹H NMR (500 MHz, CD₃CN) 8.23 ppm (1H, m), 7.95 ppm (2H, m), 7.80 ppm (7H, m), 7.43 ppm (1H, m), 7.35 ppm (1H, s), 3.30 ppm (4H, m), 2.83 ppm (4H, m).

### Example 5

Preparation of Compound 173

2-Fluoro-4-benzyloxynitrobenzene (14B):

3-Fluoro-4-nitro-phenol (1.5 g, 9.6 mmol) was dissolved in DMF with cesium carbonate (5.0 g, 15.4 mmol) and to this mixture benzyl bromide (2.0 g, 12 mmol) was added. The reaction mixture was stirred at room temperature for 3 hours, diluted with ethyl acetate, the organic layer was washed with brine, dried over magnesium sulfate, filtered and concentrated to an oil. This oil was purified by silica chromatography (5% ethyl acetate/hexane as eluent) to give 2-fluoro-4-benzyloxynitrobenzene, 1.7 g, 6.9 mmol, 72% yield of product. ¹H NMR (500 MHz, CDCl₃) 8.02 ppm (1H, t), 7.35 ppm (5H, m), 6.78 ppm (2H, m), 5.07 ppm (2H, s).

4-(5-Benzyloxy-2-nitro-phenyl)-piperazine-1-carboxylic Acid tert-butyl Ester (15B):

2-Fluoro-4-benzyloxynitrobenzene (1.7 g, 6.9 mmol) was dissolved in DMF, treated with Boc-piperazine (1.3 g, 7.0 mmol) and cesium carbonate (3.2 g, 10 mmol) and stirred at room temperature for 6 hours. The reaction mixture was diluted with ethyl acetate and the organic layer was washed with 10% citric acid, brine, dried over magnesium sulfate, filtered and concentrated to an oil. This oil was purified by silica chromatography to give 4-(5-benzyloxy-2-nitro-phenyl)-piperazine-1-carboxylic acid tert-butyl ester, 1.8 g, 4.4 mmol, 63% yield of product. ¹H NMR (500 MHz, CDCl₃) 7.86 ppm (1H, d), 7.32 ppm (5H, m), 6.48 ppm (2H, m), 5.00 ppm (2H, s), 3.48 ppm (4H, m), 2.88 ppm (4H, m) 1.36 ppm, (9H, s)

4-(2-Amino-5-benzyloxy-phenyl)-piperazine-1-carboxylic Acid tert-butyl Ester (16B):

4-(5-Benzyloxy-2-nitro-phenyl)-piperazine-1-carboxylic acid tert-butyl ester (0.22 g, 0.53 mmol) was dissolved in methylene chloride/methanol (1:1) and cooled to 0° C. To this solution NiCl₂ hexahydrate (22 mg, 0.1 mmol) was added followed by NaBH₄ (40 mg, 1 mmol). The reaction mixture was let warm to room temperature and stirred for 2 hours. An additional amount of NaBH₄ (40 mg, 1 mmol) was added and the reaction mixture was stirred for 2 hours more. At this point the solvent was removed, and the residue loaded onto a silica column with methylene chloride and eluted with 20 to 30% ethyl acetate/hexane to give 4-(2-amino-5-benzyloxy-phenyl)-piperazine-1-carboxylic acid tert-butyl ester, 0.13 g, 0.34 mmol, 68% yield of product. ¹H NMR (500 MHz, CDCl₃) 7.35 ppm (5H, m), 6.61 ppm (2H, m), 6.52 ppm (1H, m), 4.89 ppm (2H, s), 3.50 ppm (4H, br s), 2.81 ppm (4H, br s) 1.41 ppm, (9H, s), ms MH+384.2.

4-{5-Benzyloxy-2-[(naphthalene-1-carbonyl)-amino]-phenyl}-piperazine-1-carboxylic Acid tert-butyl Ester (17B):

4-(2-Amino-5-benzyloxy-phenyl)-piperazine-1-carboxylic acid tert-butyl ester (0.5 g, 1.3 mmol) was dissolved in methylene chloride with DIEA (0.35 mL, 2 mmol) and to this solution 1-naphthoyl chloride (0.25 g, 1.3 mmol) was added as a neat liquid. The reaction mixture was stirred for 2 hours, concentrated to an oil, applied to a column with methylene chloride and eluted with 20 to 30% ethyl acetate/hexanes to give 4-{5-benzyloxy-2-[(naphthalene-1-carbonyl)-amino]-phenyl}-piperazine-1-carboxylic acid tert-butyl ester as a white foam, 0.67 g, 1.2 mmol, 96% yield. ¹H NMR (500 MHz, CDCl₃) 8.74 ppm (1H, s) 8.50 ppm (1H, d), 8.38 ppm (1H, d), 7.90 ppm (1H, d), 7.81 ppm (1H, d), 7.61 ppm (1H, d), 7.3-7.5 ppm (7H, m), 6.80 ppm (1H, d), 6.74 ppm (1H, s), 4.99 ppm (2H, s), 3.45 ppm (4H, br s), 2.85 ppm (4H, br s), 1.35 ppm (9H, s).

4-{5-Hydroxy-2-[(naphthalene-1-carbonyl)-amino]-phenyl}-piperazine-1-carboxylic Acid tert-butyl Ester (18B):

4-{5-Benzyloxy-2-[(naphthalene-1-carbonyl)-amino]-phenyl}-piperazine-1-carboxylic acid tert-butyl ester (0.65 g, 1.2 mmol) was dissolved in methanol/ethyl acetate (1:1) and 10% Pd/C (0.10 g) was added. The reaction was stirred under a balloon of hydrogen (recharged several times) for 8 days. The reaction mixture was filtered through celite and concentrated to give 4-{5-hydroxy-2-[(naphthalene-1-carbonyl)-amino]-phenyl}-piperazine-1-carboxylic acid tert-butyl ester as an off-white foam, 0.52 g, 1.2 mmol, 100% yield. ¹H NMR (500 MHz, CDCl₃) 8.69 ppm (1H, s), 8.41 ppm (1H, d), 8.36 ppm (1H, m), 7.92 ppm (1H, d), 7.86 ppm (1H, m), 7.64 ppm (1H, d), 7.47 ppm (3H, m), 6.66 ppm (2H, m), 5.71 ppm (1H, s), 3.32 ppm (4H, br s), 2.72 ppm (4H, br s), 1.39 ppm (9H, s).

4-{2-[(Naphthalene-1-carbonyl)-amino]-5-trifluoromethanesulfonyloxy-phenyl}-piperazine-1-carboxylic Acid tert-butyl Ester (19B):

4-{5-Hydroxy-2-[(naphthalene-1-carbonyl)-amino]-phenyl}-piperazine-1-carboxylic acid tert-butyl ester (0.20 g, 0.45 mmol) was dissolved in methylene chloride with DIEA (0.17 mL, 1 mmol) and treated with N-phenyltrifluoromethane sulfonimide (0.178 g, 0.50 mmol). The reaction mixture was stirred at room temperature for 2 hours, concentrated and applied to a silica column, and eluted with 10% ethyl acetate/hexanes to give 4-{2-[(naphthalene-1-carbonyl)-amino]-5-trifluoromethanesulfonyloxy-phenyl}-piperazine-1-carboxylic acid tert-butyl ester as a white foam, 0.19 g, 0.33 mmol, 73% yield. ¹H NMR (500 MHz, CDCl₃) 8.90 ppm (1H, s), 8.79 ppm (1H, d), 8.31 ppm (1H, d), 7.95 ppm (1H, d), 7.84 ppm (1H, d) 7.64 ppm (1H, d), 7.45 ppm (3H, m), 7.10 ppm (1H, d), 7.00 ppm (1H, s), 3.36 ppm (4H, br s), 2.77 ppm (4H, br s) 1.38 ppm (9H, s).

Naphthalene-1-carboxylic Acid (3′,4′-dichloro-3-piperazin-1-yl-biphenyl-4-yl)-amide (Compound 173):

4-{2-[(Naphthalene-1-carbonyl)-amino]-5-trifluoromethanesulfonyloxy-phenyl}-piperazine-1-carboxylic acid tert-butyl ester (40 mg, 0.069 mmol) was placed in a screw cap test tube, dissolved in DME with potassium phosphate (80 mg, 0.38 mmol), and 3,4-dichlorophenyl boronic acid (50 mg, 0.26 mmol). To this mixture was added Pd(dppf)Cl₂ (10 mg, 0.014 mmol), argon was bubbled through for 1 min, and the reaction sealed and heated to 70° C. for 16 hours. The reaction mixture was concentrated, applied to silica with methylene chloride and eluted with 20% ethyl acetate/hexane to give the t-Boc protected product (ms MH+576). This material was dissolved in 1 mL methylene chloride and 1 mL TFA was added and the reaction mixture let stand for 1 hr. The solvent was then removed and the residue purified by reverse-phase HPLC. Fractions containing the product were concentrated to give naphthalene-1-carboxylic acid (31,41-dichloro-3-piperazin-1-yl-biphenyl-4-yl)-amide as the TFA salt, 15 mg, 0.022 mmol, 32% yield, ms MH+476.2. ¹H NMR (500 MHz, CD₃OD) 8.36 ppm (2H, m), 8.10 ppm (1H, d), 7.95 ppm (1H, d), 7.80 ppm (2H, m), 7.61 ppm (7H, m) 3.25 ppm (8H, m).

### Example 6

Preparation of Compound 176

4-(4-Bromo-2-ethoxycarbonyl-6-nitro-phenyl)-piperazine-1-carboxylic Acid tert-butyl Ester (20B):

5-Bromo-2-chloro-benzoic acid ethyl ester (19.4 g, 73.8 mmol) was dissolved in 130 ml concentrated sulfuric acid and cooled to 0° C. To this solution, potassium nitrate (8.0 g, 79 mmol) was added as a solid. The reaction mixture was stirred at 0° C. for 1 hour, poured into ice, and extracted with ethyl acetate. The organic layer was washed with brine, dried and concentrated to an oil. This oil was dissolved in DMF and Boc-piperazine (10 g, 53.8 mmol) and cesium carbonate (20 g, 62 mmol) were added. The reaction mixture was heated to 70° C. for 2 hours, let cool to room temperature, diluted with ethyl acetate, and the organic layer washed with water, 10% citric acid, brine, dried over magnesium sulfate, filtered and concentrated to an oil. The product was purified by silica chromatography to give 4-(4-bromo-2-ethoxycarbonyl-6-nitro-phenyl)-piperazine-1-carboxylic acid tert-butyl ester, 6.5 g, 14.2 mmol, 26% yield. ¹H NMR (500 MHz, CDCl₃) 7.89 ppm (1H, s), 7.85 (1H, s), 4.45 ppm (2H, q), 3.51 ppm (4H, m), 3.06 ppm (4H, m) 1.51 ppm (9H, s).

4-(4-Bromo-2-hydroxymethyl-6-nitro-phenyl)-piperazine-1-carboxylic Acid tert-butyl Ester (21B):

4-(4-Bromo-2-ethoxycarbonyl-6-nitro-phenyl)-piperazine-1-carboxylic acid tert-butyl ester (1.05 g, 2.3 mmol) was dissolved in THF and cooled to −78° C. To this solution 7 ml of a 1M solution of diisobutyl aluminum hydride in hexanes was added. The reaction mixture was then let warm to room temperature and stirred overnight. The reaction was quenched with a solution of sodium potassium tartrate, and then diluted with ethyl acetate. The organic layer was washed with a solution of sodium, potassium tartrate, dried over magnesium sulfate, filtered and concentrated to an oil. The product was purified by silica chromatography to give 4-(4-bromo-2-hydroxymethyl-6-nitro-phenyl)-piperazine-1-carboxylic acid tert-butyl ester, 0.27 g, 0.65 mmol, 28% yield. ¹H NMR (500 MHz, CDCl₃) 7.77 ppm (1H, s), 7.58 ppm, (1H, s), 4.76 ppm (2H, s), 3.8 ppm (4H, br s), 2.90 ppm (4H, br s), 1.42 ppm (9H, s).

4-[4-Bromo-2-nitro-6-(2-trifluoromethyl-phenoxymethyl)-phenyl]-piperazine-1-carboxylic Acid tert-butyl Ester (22B):

4-(4-Bromo-2-hydroxymethyl-6-nitro-phenyl)-piperazine-1-carboxylic acid tert-butyl ester (0.27 g, 0.65 mmol) was dissolved in methylene chloride with DIEA (0.35 mL, 2 mmol), cooled to 0° C. and methanesulfonyl chloride (114 mg, 1 mmol) was added as a neat liquid. The reaction was let warm to room temperature and stirred for 2 hours. Additional DIEA (0.35 mL, 2 mmol), and methane sulfonyl chloride (190 mg, 1.5 mmol) was added and the reaction mixture stirred overnight, then diluted with methylene chloride, and washed with cold 0.1N HCl and brine. The organic layer was dried over magnesium sulfate, filtered and concentrated to an oil. This oil was dissolved in acetone and 2-trifluoromethylphenol (0.32 g, 2 mmol) and potassium carbonate (0.42 g, 3 mmol) were added. The reaction mixture was stirred at room temperature for 4 days and then diluted with ethyl acetate. The organic layer was washed with water, brine, dried over magnesium sulfate, filtered and concentrated to an oil which was purified by silica chromatography to give 4-[4-bromo-2-nitro-6-(2-trifluoromethyl-phenoxymethyl)-phenyl]-piperazine-1-carboxylic acid tert-butyl ester as a yellow foam, 0.27 g, 0.48 mmol, 74% yield. ¹H NMR (500 MHz, CDCl₃) 7.92 ppm (1H, s), 7.68 ppm, (1H, s), 7.54 ppm (1 h, d) 7.43 ppm (1H, t), 7.01 ppm (1H, t), 6.92 ppm (1H, d) 5.23 ppm (2H, s), 3.95 ppm (2H, br s), 3.10 (6H, br s), 1.41 ppm (9H, s).

4-[2-Amino-4-bromo-6-(2-trifluoromethyl-phenoxymethyl)-phenyl]-piperazine-1-carboxylic Acid tert-butyl Ester (23B):

4-[4-Bromo-2-nitro-6-(2-trifluoromethyl-phenoxymethyl)-phenyl]-piperazine-1-carboxylic acid tert-butyl ester (0.27 g, 0.48 mmol) was dissolved in methylene chloride/methanol (1:1) with NiCl₂ hexahydrate (22 mg, 0.1 mmol) and cooled to 0° C. To this mixture, NaBH₄ (60 mg, 1.6 mmol) was added. The reaction was stirred for 1 hour at 0° C., concentrated, and the residue was applied to a silica column and eluted with 25% ethyl acetate/hexanes to give 4-[2-amino-4-bromo-6-(2-trifluoromethyl-phenoxymethyl)-phenyl]-piperazine-1-carboxylic acid tert-butyl ester as a white foam, 0.22 g, 0.41 mmol, 86% yield. ¹H NMR (500 MHz, CDCl₃) 7.51 ppm (1H, d), 7.40 ppm, (1H, t), 6.90 ppm (4H, m), 4.98 ppm (2H, s), 4.15 ppm (2H, br s), 3.72 (2H, br s), 3.20 ppm (2H, m), 2.95 ppm (4H, m), 1.39 ppm (9H, s).

4-[4-Bromo-2-[(isoquinoline-1-carbonyl)-amino]-6-(2-trifluoromethyl-phenoxymethyl)-phenyl]-piperazine-1-carboxylic Acid tert-butyl Ester (24B):

4-[2-Amino-4-bromo-6-(2-trifluoromethyl-phenoxymethyl)-phenyl]-piperazine-1-carboxylic acid tert-butyl ester (0.13 g, 0.25 mmol) was dissolved in DMF with 1-carboxyisoquinoline (0.17 g, 1.0 mmol), PyBOP (0.52 g, 1.0 mmol), and DIEA (0.35 mL, 2 mmol). The reaction mixture was stirred at room temperature for 3 days, diluted with ethyl acetate and the organic layer washed with water and then brine. The organic layer was dried over magnesium sulfate, filtered, and purified by silica column to give 4-[4-bromo-2-[(isoquinoline-1-carbonyl)-amino]-6-(2-trifluoromethyl-phenoxymethyl)-phenyl]-piperazine-1-carboxylic acid tert-butyl ester as a light yellow foam, 0.16 g, 0.23 mmol, 92%. ¹H NMR (500 MHz, CDCl₃) 9.70 ppm (1H, d), 8.89 ppm (1H, s), 8.50 ppm (1H, d), 7.82 ppm (2H, m), 7.70 ppm (2H, m), 7.58 ppm (1H, d), 7.51 ppm (1H, t), 7.32 ppm, (1H, s), 6.99 (2H, m), 5.08 ppm, (2H, s), 4.00 ppm (2H, br s), 3.38 ppm, (2H, m), 3.1 7 ppm, (2H, m), 2.96 ppm (2H, m), 1.46 ppm (9H, s).

Isoquinoline-1-carboxylic acid [5-bromo-2-piperazin-1-yl-3-(2-trifluoromethyl-phenoxymethyl)-phenyl]-amide (Compound 176):

4-[4-Bromo-2-[(isoquinoline-1-carbonyl)-amino]-6-(2-trifluoromethyl-phenoxymethyl)-phenyl]-piperazine-1-carboxylic acid tert-butyl ester (35 mg, 0.051 mmol) was dissolved in 1 mL methylene chloride and 1 mL TFA added. The solution was allowed to stand for one hour, concentrated to an oil and purified by reverse-phase HPLC to give isoquinoline-1-carboxylic acid [5-bromo-2-piperazin-1-yl-3-(2-trifluoromethyl-phenoxymethyl)-phenyl]-amide as a TFA salt, 10 mg, 0.014 mmol, 27% yield. ms MH+585.2, ¹H NMR (500 MHz, CDCl₃) 9.72 ppm (1H, d), 8.92 ppm (1H, s), 8.65 ppm (1H, d), 7.92 ppm (2H, m), 7.78 ppm (2H, m), 7.65 ppm (2H, m), 7.41 ppm (1H, s), 7.20 ppm (1H, d), 7.08 (1H, t), 5.18 ppm (2H, s), 3.71 ppm (2H, m), 3.66 ppm (2H, m), 3.56 ppm (2H, m), 3.40 ppm (2H, m).

### Example 7

Preparation of Compound 178

(2-Bromo-5-fluoro-phenyl)-methanol (25B):

2-Bromo-5-fluoro-benzoic acid (2.8 g, 12. 8 mmol) was dissolved in THF at 0° C. and 25 ml of a 1M solution of borane in THF was added. The reaction mixture was heated to reflux for 16 hours, cooled to room temperature, and poured into ethyl acetate and 1N HCl. The organic layer was washed with 1N NaOH, brine, dried over magnesium sulfate, filtered, and concentrated to give (2-bromo-5-fluoro-phenyl)-methanol as a white solid, 1.7 g, 8.3 mmol, 65%. ¹H NMR (500 MHz, CDCl₃) 7.47 ppm (1H, m), 7.27 ppm (1H, m), 6.85 ppm (1H, m), 4.69 ppm (2H, s).

2,2-Dimethyl-propionic Acid 2-bromo-5-fluoro-benzyl Ester (26B):

(2-Bromo-5-fluoro-phenyl)-methanol (0.79 g, 3.8 mmol) was dissolved in methylene chloride with DIEA (1 mL, 5.7 mmol), treated with about 5 mg of dimethylaminopyridine, and the solution was cooled to 0° C. and pivaloyl chloride (0.7 mL, 5.7 mmol) was added. The reaction mixture was allowed to warm to room temperature and stirred for 3 hours. The solvent was removed; the residue was dissolved in ethyl acetate and the organic layer was washed with 1N HCl, saturated sodium bicarbonate, and brine, dried over magnesium sulfate, filtered and concentrated to an oil. The product was purified by silica chromatography (5% ethyl acetate/hexanes as eluent) to give 2,2-dimethyl-propionic acid 2-bromo-5-fluoro-benzyl ester as a colorless oil, 0.88 g, 3.0 mmol, 80% yield. ¹H NMR (500 MHz, CDCl₃) 7.46 ppm (1H, m), 7.00 ppm (1H, m), 6.83 ppm (1H, m), 5.03 ppm (2H, s), 1.19 ppm (9H, s).

(2-Bromo-5-fluoro-4-nitro-phenyl)-methanol (27B):

2,2-Dimethyl-propionic acid 2-bromo-5-fluoro-benzyl ester (5.0 g, 17.3 mmol) was dissolved in 50 ml concentrated sulfuric acid and cooled to 0° C. Potassium nitrate (1.7 g, 17.3 mmol) was added as a solid and the reaction stirred at 0° C. for 2 hours and then poured into ice and extracted with ethyl acetate. The organic layer was washed with brine, dried over magnesium sulfate, filtered, and concentrated to an oil. The product was purified by silica chromatography (20% ethyl acetate/hexanes) to give (2-bromo-5-fluoro-4-nitro-phenyl)-methanol as a beige solid, 2.6 g, 10.4 mmol, 60% yield. ¹H NMR (500 MHz, CDCl₃) 8.20 ppm (1H, d), 7.52 ppm (1H, d), 4.71 ppm (2H, s).

4-(4-Bromo-5-hydroxymethyl-2-nitro-phenyl)-piperazine-1-carboxylic Acid tert-butyl Ester (28B):

(2-Bromo-5-fluoro-4-nitro-phenyl)-methanol (2.6 g, 10.4 mmol) was dissolved in DMF with t-Boc-piperazine (3.3 g, 17.7 mmol and cesium carbonate (6.5 g, 20 mmol). The reaction mixture became purple and was then stirred overnight and poured into ethyl acetate/water. The organic layer was washed with 10% citric acid, brine, dried over magnesium sulfate, filtered, and concentrated to give 4-(4-bromo-5-hydroxymethyl-2-nitro-phenyl)-piperazine-1-carboxylic acid tert-butyl ester as an oil, 3.0 g, 7.2 mmol, 69% yield. ¹H NMR (500 MHz, CDCl₃) 7.80 ppm (1H, s), 7.15 ppm (1H, s), 4.52 ppm (2H, s), 3.32 ppm (4H, m), 2.80 ppm (4H, m), 2.42 ppm (1H, m), 1.25 ppm (9H, s).

4-(4-Bromo-5-bromomethyl-2-nitro-phenyl)-piperazine-1-carboxylic Acid tert-butyl Ester (29B):

4-(4-Bromo-5-hydroxymethyl-2-nitro-phenyl)-piperazine-1-carboxylic acid tert-butyl ester (80 mg, 0.19 mmol) was dissolved in methylene chloride and carbon tetrabromide (70 mg, 0.21 mmol) and triphenyl phosphine (55 mg, 0.21 mmol) were added as solids. The reaction mixture was stirred for 2 hours and then applied directly to a silica column and eluted with 10% ethyl acetate/hexanes to give after solvent removal 4-(4-bromo-5-bromomethyl-2-nitro-phenyl)-piperazine-1-carboxylic acid tert-butyl ester as an orange solid, 75 mg, 0.16 mmol, 83% yield. ¹H NMR (500 MHz, CDCl₃) 7.98 ppm (1H, s), 7.11 ppm (1H, s), 4.47 ppm (2H, s), 3.55 ppm, (4H, m), 2.98 ppm (4H, br s), 1.42 ppm (9H, s).

4-[4-Bromo-2-nitro-5-(2-trifluoromethyl-phenoxymethyl)-phenyl]-piperazine-1-carboxylic Acid tert-butyl Ester (30B):

4-(4-Bromo-5-bromomethyl-2-nitro-phenyl)-piperazine-1-carboxylic acid tert-butyl ester (1.0 g, 2.1 mmol) was dissolved in DMF with 2-trifluoromethyl phenol (1.0 g, 6.2 mmol) and cesium carbonate (2.0 g, 6.2 mmol). The reaction mixture was stirred for 4 hours at room temperature, diluted with ethyl acetate and the organic layer washed with 1N NaOH, brine, dried over magnesium sulfate, filtered, and concentrated to give 4-[4-bromo-2-nitro-5-(2-trifluoromethyl-phenoxymethyl)-phenyl]-piperazine-1-carboxylic acid tert-butyl ester as an orange oil. The product was purified by silica chromatography (10% ethyl acetate/hexanes) to give an orange oil, 0.95 g, 1.7 mmol, 81% yield. ¹H NMR (500 MHz, CDCl₃) 7.99 ppm (1H, s), 7.55 ppm (1H, d), 7.51 ppm (1H, s), 7.49 ppm (1H, m), 7.00 ppm (2H, m) 5.06 ppm, (2H, s), 3.50 ppm (4H, m), 2.98 ppm (4H, br s), 1.40 ppm (9H, s).

4-[2-Amino-4-bromo-5-(2-trifluoromethyl-phenoxymethyl)-phenyl]-piperazine-1-carboxylic Acid tert-butyl Ester (31B):

4-[4-Bromo-2-nitro-5-(2-trifluoromethyl-phenoxymethyl)-phenyl]-piperazine-1-carboxylic acid tert-butyl ester (0.95 g, 1.7 mmol) was dissolved in 10 mL of DMF and tin chloride dihydrate (1.9 g, 8.5 mmol) was added as a solid. The reaction mixture was stirred at room temperature overnight and then poured into 1N NaOH. The aqueous layer was extracted with ethyl acetate. The organic layer was washed with brine, dried over magnesium sulfate, filtered and concentrated to an oil. The product was purified by silica chromatography (20% ethyl acetate/hexanes) to give 4-[2-amino-4-bromo-5-(2-trifluoromethyl-phenoxymethyl)-phenyl]-piperazine-1-carboxylic acid tert-butyl ester as a white solid, 0.76 g, 1.4 mmol, 84% yield. ¹H NMR (500 MHz, CDCl₃) 7.55 ppm (1H, d), 7.44 ppm (1H, t), 7.19 ppm (1H, s), 6.98 ppm (1H, d), 6.90 ppm (1H, t), 5.04 (2H, s), 3.95 ppm (2H, br s), 3.50 ppm (4H, br s), 2.87 ppm (4H, br s), 1.40 ppm (9H, s).

4-[4-Bromo-2-[(isoquinoline-1-carbonyl)-amino]-5-(2-trifluoromethyl-phenoxymethyl)-phenyl]-piperazine-1-carboxylic Acid tert-butyl Ester (32B):

4-[2-Amino-4-bromo-5-(2-trifluoromethyl-phenoxymethyl)-phenyl]-piperazine-1-carboxylic acid tert-butyl ester (0.30 g, 0.57 mmol) was dissolved in DMF with 1-carboxyisoquinoline (0.17 g, 1 mmol) and HBTU (0.38 g, 1 mmol). To this solution DIEA (0.4 mL, 2.3 mmol) was added and the reaction mixture stirred at room temperature overnight. The reaction mixture was then diluted with ethyl acetate and the organic layer washed with saturated sodium bicarbonate, brine, dried over magnesium sulfate, filtered and concentrated to a brown solid. The product was purified by silica chromatography (16% ethyl acetate/hexanes) to give 4-[4-bromo-2-[(isoquinoline-1-carbonyl)-amino]-5-(2-trifluoromethyl-phenoxymethyl)-phenyl]-piperazine-1-carboxylic acid tert-butyl ester as a yellow solid, 0.32 g, 0.47 mmol, 82% yield. ¹H NMR (500 MHz, CDCl₃) 9.66 ppm (1H, d) 8.88 ppm (1H, s), 8.50 ppm (1H, s), 7.81 ppm (2H, m), 7.65 ppm (2H m), 7.53 ppm (1H, d), 7.41 ppm (1H, m), 7.40 ppm (1H, s), 7.01 ppm (1H, d), 6.93 ppm, (1H, t), 5.12 ppm (2H, s), 3.65 ppm (4H, br s), 2.82 ppm (4H, br s), 1.41 ppm (9H, s).

Isoquinoline-1-carboxylic Acid [5-bromo-2-piperazin-1-yl-4-(2-trifluoromethyl-phenoxymethyl)-phenyl]-amide (Compound 178):

4-[4-Bromo-2-[(isoquinoline-1-carbonyl)-amino]-5-(2-trifluoromethyl-phenoxymethyl)-phenyl]-piperazine-1-carboxylic acid tert-butyl ester (30 mg, 0.044 mmol) was dissolved in 1 ml methylene chloride and 1 ml TFA added. After one hour the reaction mixture was concentrated to an oil and the product crystallized from methanol/Et₂O to give isoquinoline-1-carboxylic acid [5-bromo-2-piperazin-1-yl-4-(2-trifluoromethyl-phenoxymethyl)-phenyl]-amide as a yellow solid as the TFA salt, 20 mg, 0.029 mmol, 66% yield. LC/ms, ret time 3.38 min, MH+585.1. ¹H NMR (500 MHz, CD₃OD) 9.54 ppm (1H, d), 8.65 ppm (1H, d), 8.50 ppm (1H, d), 8.06 ppm (2H, m), 7.81 ppm (2H, m), 7.60 ppm (2H, m), 7.48 ppm (1H, s), 7.39 ppm (1H, d), 7.25 ppm (1H, d), 7.08 ppm (1H, t), 5.26 ppm (2H, s), 3.55 ppm (4H, m), 3.25 ppm (4H, m)

### Example 8

Preparation of Compound 186

Isoquinoline-1-carboxylic Acid [4′-hydroxy-4-piperazin-1-yl-6-(2-trifluoromethyl-phenoxymethyl)-biphenyl-3-yl]-amide (Compound 186):

4-[4-Bromo-2-[(isoquinoline-1-carbonyl)-amino]-5-(2-trifluoromethyl-phenoxymethyl)-phenyl]-piperazine-1-carboxylic acid tert-butyl ester (50 mg, 0.073 mmol) was placed in a screw cap test tube and dissolved in DME with potassium phosphate (80 mg, 0.38 mmol), and 4-(4,4,5,5-tetramethyl-[1,3,2]dioxaborolan-2-yl)-phenol (60 mg, 0.27 mmol). To this mixture was added Pd(dppf)Cl₂ (10 mg, 0.014 mmol), argon was bubbled through for 1 min, and the reaction sealed and heated to 70° C. for 16 hours. The reaction mixture was diluted with ethyl acetate, filtered, and the filtrate concentrated to an oil which was purified by silica chromatography (33% ethyl acetate/hexane eluent) to give the t-boc protected product. This product was dissolved in methylene chloride and treated with TFA. After one hour the solvent was removed and the product crystallized from methanol/Et₂O to give isoquinoline-1-carboxylic acid [4′-hydroxy-4-piperazin-1-yl-6-(2-trifluoromethyl-phenoxymethyl)-biphenyl-3-yl]-amide as a yellow solid 15 mg, 0.021 mmol, 29% yield. ¹H NMR (500 MHz, CD₃OD) 9.58 ppm (1H, d), 8.65 ppm (1H, d), 8.48 ppm (1H, s), 8.02 ppm (2H, m), 7.80 ppm (2H, m), 7.78 ppm (1H, m), 7.59 ppm (2H, m), 7.46 ppm (1H, t), 7.29 ppm (2H, d), 7.03 ppm (1H, t), 6.94 ppm (1H, m), 6.89 ppm (2H, d), 5.10 (2H, s), 3.58 ppm (4H, m), 3.28 ppm (4H, m). ms MH+599.2.

### Example 9

Preparation of Compound 200

Piperazine-1,2,4-tricarboxylic acid 1-benzyl Ester 4-tert-butyl Ester (33B):

Piperazine-1,3-dicarboxylic acid 1-tert-butyl ester (1.12 g, 4.87 mmol) was dissolved in 20 ml 50% acetone in water at OC. To this solution was added sodium bicarbonate and benzyl chloroformate (0.91 g, 5.36 mmol). The reaction mixture was stirred for eighteen hours, filtered and the organic layer was removed under reduced pressure. The aqueous layer was extracted with ethyl ether, the organics were washed with HCl (0.5N), brine and then dried over magnesium sulfate, filtered and concentrated under reduced pressure to give piperazine-1,2,4-tricarboxylic acid 1-benzyl ester 4-tert-butyl ester as a clear oil, 1.60 g, 4.39 mmol, 92%. ¹H NMR (500 MHz, CDCl₃) 7.35 ppm (5H, m), 5.18 ppm (2H, m), 4.75 ppm (2H, m), 3.90 ppm (2H, m), 3.20 ppm (2H, m), 2.85 ppm (1H, m), 1.48 ppm (9H, s).

2-(Naphthalen-2-ylcarbamoyl)-piperazine-1,4-dicarboxylic acid 1-benzyl Ester 4-tert-butyl Ester (34B):

Piperazine-1,2,4-tricarboxylic acid 1-benzyl ester 4-tert-butyl ester (0.15 g 0.41 mmol) was dissolved in 5 ml of methylene chloride and to this solution was added EDC (0.09 g 0.45 mmol), DIEA (0.16 g, 1.35 mmol), HOBt (0.07 g 0.45 mmol) and naphthalen-2-ylamine (0.29 g, 2.25 mmol). The reaction mixture was stirred for eighteen hours. The resulting solution was diluted with ethyl acetate and the organic layer was washed with HCl (0.5N), brine and then dried over magnesium sulfate, filtered and concentrated under reduced pressure to yield a brown oil which was taken up in 5.0 ml 0.1% TFA acetonitrile and filtered. The filtrate was then purified by HPLC (with a gradient 50-100% acetonitrile/water) to give 2-(naphthalen-2-ylcarbamoyl)-piperazine-1,4-dicarboxylic acid 1-benzyl ester 4-tert-butyl ester as a yellow solid 0.08 g, 0.16 mmol, 39%. ¹H NMR(500 MHz, CDCl₃) 8.65 ppm (1H, s), 7.75 ppm (3H, m), 7.40 ppm (5H, m), 7.10 ppm (2H, m), 6.70 ppm (2H, m), 5.20 ppm (2H, m), 4.70 ppm (2H, m), 3.95 ppm (2H, m), 3.15 ppm (2H, m), 2.80 ppm (1H, m), 1.48 ppm (9H, s).

4-[4-(4-Chloro-2-methyl-phenoxy)-butyryl]-3-(naphthalen-2-ylcarbamoyl)-piperazine-1-carboxylicacidtert-butyl Ester (35B):

2-(Naphthalen-2-ylcarbamoyl)-piperazine-1,4-dicarboxylic acid 1-benzyl ester 4-tert-butyl ester, 0.08 g, 0.16 mmol., was dissolved in 15 ml methanol and purged with nitrogen. Palladium, 10 wt. % on activated carbon (0.03 g), was added and the reaction mixture was subjected to a hydrogen atmosphere for three hours. The reaction was degassed with nitrogen and filtered, and the resulting filtrate was evaporated and dried under high vacuum to give 3-(naphthalen-2-ylcarbamoyl)-piperazine-1-carboxylic acid tert-butyl ester as a yellow oil, 0.06 g, 0.16 mmol. 3-(naphthalen-2-ylcarbamoyl)-piperazine-1-carboxylic acid tert-butyl ester (0.06 g 0.16 mmol) was added to a solution of EDC (0.09 g 0.45 mmol), DIEA (0.19 g, 1.5 mmol), DMAP (0.03 g 0.3 mmol) and 4-(4-chloro-2-methyl-phenoxy)-butyric acid (0.10 g, 0.45 mmol) in 5 ml of methylene chloride, and the reaction mixture was stirred for eighteen hours. The resulting solution was diluted with ethyl acetate, the organic layer was separated and washed with HCl (0.5N) and brine, and then dried over magnesium sulfate. Filtration and concentration under reduced pressure provided a brown oil which was taken up in 5.0 ml 0.1% TFA acetonitrile and filtered. The filtrate was then purified by HPLC (with a gradient 50-100% acetonitrile/water) to yield 4-[4-(4-chloro-2-methyl-phenoxy)-butyryl]-3-(naphthalen-2-ylcarbamoyl)-piperazine-1-carboxylicacidtert-butyl ester as a yellow solid 0.04 g, 0.07 mmol, 45%. ¹H NMR (500 MHz, CDCl₃) 8.75 ppm (1H, m), 8.20 ppm (1H, m), 7.75 ppm (2H, m), 7.40 ppm (2H, m), 7.10 ppm (3H, m), 6.68 ppm (2H, m), 4.60 ppm (1H, m), 3.90 ppm (4H, m), 3.35 ppm (1H, m), 3.20 ppm (1H, m), 2.70 ppm (2H, m), 2.20 ppm (7H, m), 1.48 ppm (9H, s).

1-[4-(4-Chloro-2-methyl-phenoxy)-butyryl]-piperazine-2-carboxylic Acid naphthalen-2-ylamide (Compound 200):

4-[4-(4-Chloro-2-methyl-phenoxy)-butyryl]-3-(naphthalen-2-ylcarbamoyl)-piperazine-1-carboxylicacidtert-butyl ester, 0.04 g, 0.07 mmol, was dissolved in a solution of 20% TFA in methylene chloride and stirred at room temperature for thirty minutes. The solution was diluted with ethyl ether and the resulting crystals were collected and washed with cold ethyl ether, then dried under reduced pressure to yield 1-[4-(4-chloro-2-methyl-phenoxy)-butyryl]-piperazine-2-carboxylic acid naphthalen-2-ylamide as a white solid, 0.020 g, 0.05 mmol, 63% as the TFA salt. ¹H NMR (500 MHz, CDCl₃) 8.65 ppm (1H, m), 8.05 ppm (1H, m), 7.78 ppm (2H, m), 7.43 ppm (2H, m), 7.05 ppm (3H, m), 6.65 ppm (2H, m), 4.0 ppm (4H, m), 3.65 ppm (2H, m).3.30 ppm (2H, m), 2.65 ppm (3H, m) 2.15 ppm (5H, m).

### Example 10

Preparation of Compound 196

4-(6-Chloro-3-nitro-pyridin-2-yl)-piperazine-1-carboxylic Acid tert-butyl Ester (36B):

2,6-Dichloro-3-nitro-pyridine (1.0 g, 5.18 mmol) was dissolved in 15 ml toluene, treated with piperazine-1-carboxylic acid tert-butyl ester (0.96 g, 5.18 mmol) and stirred for four hours. The reaction mixture was applied to a silica column and eluted with 25% ethyl acetate in hexane to yield 4-(6-chloro-3-nitro-pyridin-2-yl)-piperazine-1-carboxylic acid tert-butyl ester as a yellow solid, 0.87 g, 2.54 mmol, 49%. ¹H NMR (500 MHz, CDCl₃) 8.18 ppm (1H, d), 6.78 ppm (1H, d), 3.62 ppm (4H, m), 3.50 ppm (4H, m), 1.52 ppm (9H, s).

4-[6-(3,4-Dichloro-phenyl)-3-nitro-pyridin-2-yl]-piperazine-1-carboxylic acid tert-butyl ester (37B):

The compound described above(0.2 g, 0.58 mmol) was dissolved 15 ml of DME and purged with nitrogen for five minutes. To this solution was added potassium phosphate (0.37 g, 1.75 mmol) followed by dichloro(1,1-bis (diphenylphosphine)ferrocene) palladium (II) dichloromethane adduct (0.07 g, 0.09 mmol) and the mixture was heated to 80° C. for eighteen hours. The reaction was cooled to room temperature, diluted with ethyl acetate and the organic layer was washed with saturated sodium bicarbonate, water, brine and then dried over magnesium sulfate, filtered and concentrated under reduced pressure to give a brown oil. The residue was applied to a silica column with methylene chloride and eluted with 25% ethyl acetate in hexane to yield 4-[6-(3,4-dichloro-phenyl)-3-nitro-pyridin-2-yl]-piperazine-1-carboxylic acid tert-butyl ester as a yellow solid, 0.18 g, 0.40 mmol, 68%. ¹H NMR (500 MHz, CDCl₃) 8.08 ppm (1H, d), 7.92 ppm (1H, s), 7.63 ppm (1H, d), 7.33 ppm (1H, d), 7.10 ppm (1H, d), 3.45 ppm (4H, m), 3.35 ppm (4H, m), 1.41 ppm (9H, s).

4-{6-(3,4-Dichloro-phenyl)-3-[(naphthalene-1-carbonyl)-amino]-pyridin-2-yl}-piperazine-1-carboxylic Acid tert-butyl Ester (38B):

4-[6-(3,4-Dichloro-phenyl)-3-nitro-pyridin-2-yl]-piperazine-1-carboxylic acid tert-butyl ester, 0.18 g, 0.40 mmol, was dissolved in methanol, purged with nitrogen, treated with palladium, 10 wt. % on activated carbon (0.03 g), and subjected to a hydrogen atmosphere for two hours. The reaction was again purged with nitrogen and filtered. The resulting filtrate was evaporated and dried under high vacuum to give 4-[3-amino-6-(3,4-dichloro-phenyl)-pyridin-2-yl]-piperazine-1-carboxylic acid tert-butyl ester as a clear oil, 0.18 g, 0.4 mmol.

4-[3-Amino-6-(3,4-dichloro-phenyl)-pyridin-2-yl]-piperazine-1-carboxylic acid tert-butyl ester, 0.18 g, 0.4 mmol, was dissolved in 5 ml of methylene chloride and to this solution was added TEA (0.06 g, 0.6 mmol) and 2 equivalents of 1-napthoyl chloride (0.16 g, 0.8 mmol). The resulting solution was stirred at room temperature for eighteen hours, evaporated to dryness and taken up in 5.0 ml 0.1% TFA in acetonitrile and filtered. The filtrate was then purified by HPLC (with a gradient 50-100% acetonitrile/water) to yield 4-{6-(3,4-dichloro-phenyl)-3-[(naphthalene-1-carbonyl)-amino]-pyridin-2-yl}-piperazine-1-carboxylic acid tert-butyl ester as a white solid, 0.030 g, 0.05 mmol, 15% for two steps. ¹H NMR (500 MHz, CDCl₃) 8.93 ppm (1H, d), 8.78 ppm (1H, s), 8.49 ppm (1H, d), 8.15 ppm (1H, s), 8.05 ppm (1H, d), 7.86 ppm (1H, d), 7.80 ppm (1H, d), 7.55 ppm (3H, m), 7.47 ppm (1H, m), 3.55 ppm (4H, m), 3.2 ppm (4H, m), 1.48 ppm (9H, s).

Naphthalene-1-carboxylic Acid [6-(3,4-dichloro-phenyl)-2-piperazin-1-yl-pyridin-3-yl]-amide (Compound 196):

4-{6-(3,4-Dichloro-phenyl)-3-[(naphthalene-1-carbonyl)-amino]-pyridin-2-yl}-piperazine-1-carboxylic acid tert-butyl ester, 0.030 g, 0.05 mmol, was dissolved in a solution of 20% TFA in methylene chloride and stirred at room temperature for thirty minutes. The solution was diluted with ethyl ether and the resulting crystals were collected by filtration, washed with cold ethyl ether and then dried under reduced pressure to yield 0.020 g, 0.05 mmol, 81%, of naphthalene-1-carboxylic acid [6-(3,4-dichloro-phenyl)-2-piperazin-1-yl-pyridin-3-yl]-amide as the TFA salt. ¹H NMR (500 MHz, CD₃CN) 8.80 ppm (1H, d), 8.68 ppm (1H, s), 8.37 ppm (1H, m), 8.25 ppm (1H, m), 8.80 ppm (1H, m), 8.00 ppm (2H, m), 7.80 ppm (1H, d), 7.75 ppm (1H, d), 7.60 ppm (4H, m), 3.42 ppm (4H, m), 3.3 ppm (4H, m).

### Example 11

Preparation of Compound 197

4-(2-Chloro-5-nitro-pyrimidin-4-yl)-piperazine-1-carboxylic Acid tert-butyl Ester (39B):

2,4-Dichloro-5-nitro-pyrimidine (1.0 g, 5.17 mmol) was dissolved in 15 ml methylene chloride with TEA (0.78 g, 7.75 mmol) and piperazine-1-carboxylic acid tert-butyl ester (0.96 g, 5.17 mmol) and stirred for four hours. The reaction mixture was applied directly to a silica column and eluted with 25% ethyl acetate in hexane to yield 4-(2-chloro-5-nitro-pyrimidin-4-yl)-piperazine-1-carboxylic acid tert-butyl ester as a yellow solid, 0.64 g, 1.86 mmol, 36%. ¹H NMR (500 MHz, CDCl₃) 8.87 ppm (1H, d), 3.62 ppm (8H, m), 1.48 ppm (9H, s).

4-[2-(3,4-Dichloro-phenyl)-5-nitro-pyrimidin-4-yl]-piperazine-1-carboxylic Acid tert-butyl Ester (40B):

4-(2-Chloro-5-nitro-pyrimidin-4-yl)-piperazine-1-carboxylic acid tert-butyl ester (0.1 g, 0.29 mmol) was dissolved in 15 mL of DME and purged with nitrogen for five minutes. To this solution was added potassium phosphate (0.19 g, 0.88 mmol) followed by dichloro[1,1-bis (diphenylphosphine)ferrocene] palladium (II) dichloromethane adduct (0.07 g, 0.09 mmol) and heated to 80° C. for eighteen hours. The reaction was cooled to room temperature, diluted with ethyl acetate, the organics were separated and washed with saturated sodium bicarbonate, water, brine and then dried over magnesium sulfate. The solution was filtered, concentrated under reduced pressure to give a brown oil. This was applied to a silica column with methylene chloride and eluted with 25% ethyl acetate in hexane to yield 4-[2-(3,4-dichloro-phenyl)-5-nitro-pyrimidin-4-yl]-piperazine-1-carboxylic acid tert-butyl ester as a yellow solid, 0.10 g, 0.22 mmol, 73%. ¹H NMR (500 MHz, CDCl₃) 9.02 ppm (1H, d), 8.47 ppm (1H, m), 8.20 ppm (1H, m), 7.55 ppm (1H, m), 3.65 (8H, m), 1.52 ppm (9H, s).

4-[5-Amino-2-(3,4-dichloro-phenyl)-pyrimidin-4-yl]-piperazine-1-carboxylic Acid tert-butyl Ester (41B):

4-[2-(3,4-Dichloro-phenyl)-5-nitro-pyrimidin-4-yl]-piperazine-1-carboxylic acid tert-butyl ester, 0.05 g, 0.11 mmol, was dissolved in methanol and purged with nitrogen. Palladium/10 wt. % on activated carbon (0.03 g) was added and the reaction stirred under hydrogen. After two hours the reaction was filtered and the resulting filtrate evaporated and dried under high vacuum to give a clear oil 0.05 g, 0.11 mmol.

This crude material was dissolved in 5 ml of methylene chloride and treated with TEA (0.02 g, 0.16 mmol) and 2 equivalents of 1-naphthoyl chloride (0.04 g, 0.22 mmol). The resulting solution was stirred at room temperature for eighteen hours, evaporated to dryness, taken up in 5.0 ml 0.1% TFA acetonitrile and filtered. The filtrate was then purified by HPLC (with a gradient 50-100% acetonitrile/water) to yield 4-[5-amino-2-(3,4-dichloro-phenyl)-pyrimidin-4-yl]-piperazine-1-carboxylic acid tert-butyl ester as a white solid, 0.010 g, 0.02 mmol, 16% for two steps. ¹H NMR (500 MHz, CDCl₃) 9.61 ppm (1H, m), 8.82 ppm (1H, m), 8.47 ppm (1H, m), 8.23 ppm (1H, s), 8.05 ppm (1H, d), 7.95 ppm 7.86 ppm (2H, m), 7.62 ppm (1H, d), 7.55 ppm (3H, m), 4.03 ppm (4H, m), 3.60 ppm (4H, m), 1.48 ppm (9H, s).

Naphthalene-1-carboxylic Acid [2-(3,4-dichloro-phenyl)-4-piperazin-1-yl-pyrimidin-5-yl]-amide (Compound 197)

4-{2-(3,4-Dichloro-phenyl)-5-[(naphthalene-1-carbonyl)-amino]-pyrimidin-4-yl}-piperazine-1-carboxylic acid tert-butyl ester, 0.010 g, 0.02 mmol, was dissolved in a solution of 20% TFA in methylene chloride and stirred at room temperature for thirty minutes. The product was precipitated in crystalline form by diluting the reaction mixture with ethyl ether. The crystals were collected and washed with cold ethyl ether then dried under reduced pressure to yield naphthalene-1-carboxylic acid [2-(3,4-dichloro-phenyl)-4-piperazin-1-yl-pyrimidin-5-yl]-amide, 0.01 g, 0.02 mmol 81% as the TFA salt. ¹H NMR (500 MHz, CD₃CN) 8.83 ppm (1H, m), 8.62 ppm (1H, m), 8.55 ppm (1H, m), 8.42 ppm (1H, m), 8.35 ppm (1H, m), 8.11 ppm (1H, m), 8.02 ppm (1H, m), 7.88 ppm (1H, m), 7.70 ppm (4H, m), 4.02 ppm (4H, m), 3.32 ppm (4H, m).

### Example 12

Preparation of Compound 201

4-(2-Amino-4-trifluoromethyl-phenyl)-[1,4]diazepane-1-carboxylic Acid tert-butyl Ester (42B):

4-(2-Nitro-4-trifluoromethyl-phenyl)-[1,4]diazepane-1-carboxylic acid tert-butyl ester (0.10 g, 0.26 mmol) was dissolved in 10 ml THF and purged with nitrogen. Palladium, 10 wt. % on activated carbon (0.30 g), was added and the mixture subjected to hydrogen for three hours. The reaction was again purged with nitrogen and filtered. The resulting filtrate was evaporated and dried under high vacuum to give 4-(2-amino-4-trifluoromethyl-phenyl)-[1,4]diazepane-1-carboxylic acid tert-butyl ester as a yellow solid 0.92 g, 0.26 mmol, 100%. ¹H NMR (500 MHz, CDCl₃) 7.75 ppm (1H, s), 7.45 ppm (1H, m), 7.12 ppm (1H, m), 4.20 ppm (2H, m) 3.56 ppm (8H, m) 1.96 ppm (2H, m), 1.48 ppm (9H, s).

4-{2-[(Naphthalene-1-carbonyl)-amino]-4-trifluoromethyl-phenyl}-[1,4]diazepane-1-carboxylic Acid tert-butyl Ester (43B):

4-(2-Amino-4-trifluoromethyl-phenyl)-[1,4]diazepane-1-carboxylic acid tert-butyl ester (0.02 g 0.06 mmol) was dissolved in 5 ml of methylene chloride and to this solution was added TEA (0.01 g, 0.09 mmol) and 2 equivalents of 1-naphthoyl chloride (0.02 g, 0.12 mmol). The resulting solution was stirred at room temperature for eighteen hours, evaporated to dryness and the residue was applied to a silica column with methylene chloride and eluted with 20% ethyl acetate in hexanes to yield 4-{2-[(naphthalene-1-carbonyl)-amino]-4-trifluoromethyl-phenyl}-[1,4]diazepane-1-carboxylic acid tert-butyl ester as a yellow solid, 0.02 g, 0.04 mmol, 73%. ¹H NMR (500 MHz, CDCl₃) 9.13 ppm (1H, m), 9.00 ppm (1H, m), 8.48 ppm (1H, m), 8.05 ppm (1H, m), 7.94 ppm (1H, m), 7.78 ppm (1H, m), 7.55 ppm (3H, m), 7.38 ppm (1H, m), 7.28 ppm (1H, m), 3.46 ppm (4H, m), 3.10 ppm (4H, m), 1.80 ppm (2H, m), 1.43 ppm (9H, s).

Naphthalene-1-carboxylic Acid (2-[1,4]diazepan-1-yl-5-trifluoromethyl-phenyl)-amide (Compound 201):

4-{2-[(Naphthalene-1-carbonyl)-amino]-4-trifluoromethyl-phenyl}-[1,4]diazepane-1-carboxylic acid tert-butyl ester, 0.02 g, 0.04 mmol., was dissolved in a solution of 20% TFA in methylene chloride solution and stirred at room temperature for thirty minutes. The solution was diluted with ethyl ether the resulting crystals were collected and washed with cold ethyl ether then dried under reduced pressure to yield naphthalene-1-carboxylic acid (2-[1,4]diazepan-1-yl-5-trifluoromethyl-phenyl)-amide, 0.020 g, 0.05 mmol, 64% as the TFA salt. ¹H NMR (500 MHz, CD₃CN) 8.85 ppm (1H, m), 8.71 ppm (1H, m), 8.37 ppm (1H, m), 8.25 ppm (1H, m), 8.12 ppm (1H, m), 7.78 ppm (1H, m), 7.55 ppm (4H, m), 7.38 ppm (1H, m), 3.32 ppm (4H, m), 2.85 ppm (4H, m), 1.95 ppm (2H, m)

### Example 13

Preparation of Compound 250

1-Benzyl-3-hydroxymethyl-piperidin-4-ol (44B):

To a solution of 1-benzyl-4-oxo-piperidine-3-carboxylic acid methyl ester (22.16 g, 0.47 mol) in THF (300 ml) at 0° C. was added dropwise a 1N solution of lithium aluminum hydride in THF (300 ml, 0.3 mol). After stirring at RT for 1 h, the reaction was heated at 80° C. for 2 h. After cooling to RT, the reaction was poured into 500 g of Na₂SO₄.10H₂O. Filtration, washing with dichloromethane and evaporation then gave crude 1-benzyl-3-hydroxymethyl-piperidin-4-ol (16.42 g) that was used directly for the next step.

1-Benzyl-3-(tert-butyl-diphenyl-silanyloxymethyl)-piperidin-4-ol (45B):

To a solution of 1-benzyl-3-hydroxymethyl-piperidin-4-ol (16.40 g, 74 mmol), chloro-(t-butyl)diphenyl-silane (22.41 g, 81.5 mmol)and triethylamine (12.4 ml, 89 mmol) in dichloromethane (200 ml) was added 4-N-dimethylaminopyridine (100 mg) and the resulting mixture was stirred at RT for 7 days. The reaction was washed with water (200 ml) and dried (Na₂SO₄). Evaporation and purification of the residue by flash chromatography (SiO₂, 5% to 30% ethyl acetate in hexane) gave 1-benzyl-3-(tert-butyl-diphenyl-silanyloxymethyl)-piperidin-4-ol (3.47 g).

1-Benzyl-3-(tert-butyl-diphenyl-silanyloxymethyl)-piperidin-4-one (46B):

To a solution of oxalyl chloride (1.0 ml, 11.5 mmol) in dichloromethane (50 ml) at −78° C. was added a solution of DMSO (1.6 ml, 22.5 mmol) in dichloromethane (5 ml) and the resulting solution was stirred at same temperature for 15 min. A pre-cooled solution of 1-benzyl-3-(tert-butyl-diphenyl-silanyloxymethyl)-piperidin-4-ol (3.46 g, 7.53 mmol) in dichloromethane (10 ml) was added at −78° C. After 40 min at same temperature, triethylamine (7 ml, 50 mmol) was added. The reaction was brought to RT, washed with water (20 ml), dried and evaporated. Purification of the crude product by flash column (SiO₂, 5% ethyl acetate/hexane) then gave 1-benzyl-3-(tert-butyl-diphenyl-silanyloxymethyl)-piperidin-4-one (3.14 g, 91%). ¹H—NMR (500 MHz, CDCl₃): δ 7.65-7.30 (m, 15H), 3.99 (dd, 1H), 3.75 (dd, 1H), 3.64 (dd, 2H), 3.33.30-3,25 (m, 1H), 3.02-2.97 (m, 1H), 2.83-2.76 (m, 1H), 2.57-2.44 (m, 2H), 2.30 (m, 2H), 0.98 (s, 9H).

(1-Benzyl-4-biphenyl-4-yl-1,2,3,6-tetrahydro-pyridin-3-yl)-methanol (47B):

To 1-benzyl-3-(tert-butyl-diphenyl-silanyloxymethyl)-piperidin-4-one (1.27 g, 2.8 mmol) in diethyl ether (20 ml) at −78° C. was added a 0.5 M solution of 4-phenylphenylmagnesium chloride (10 ml, 5 mmol) in THF. After 3 h, the reaction was brought to RT, evaporated and mixed with water (100 ml) and ammonium chloride (1 g). Extraction with ethyl ether (3×40 ml), drying (Na₂SO₄) and concentration under vacuum gave 1-benzyl-4-biphenyl-4-yl-3-(tert-butyl-diphenyl-silanyloxymethyl)-piperidin-4-ol, which was mixed with trifluoroacetic acid (20 ml) and heated under reflux for 20 h. After removal of TFA, saturated aqueous potassium bicarbonate solution (100 ml) was added. Extraction with dichloromethane (3×40 ml), drying, and concentration gave the crude product, which was purified by column (SiO₂, 10% to 40% ethyl acetate in hexane) to afford (1-benzyl-4-biphenyl-4-yl-1,2,3,6-tetrahydro-pyridin-3-yl)-methanol (50% from 1-benzyl-3-(tert-butyl-diphenyl-silanyloxymethyl)-piperidin-4-one).

1-Benzyl-4-biphenyl-4-yl-3-(naphthalen-2-yloxymethyl) 1,2,3,6-tetrahydro-pyridine (48B):

To a solution of (1-benzyl-4-biphenyl-4-yl-1,2,3,6-tetrahydro-pyridin-3-yl)-methanol(5) (0.226 g, 0.66 mmol) in dichloromethane (2 ml) at 0° C. was added methylsulfonyl chloride (0.0984 ml, 1.27 mmol) and triethylamine (0.177 ml, 1.28 mmol. The reaction was brought to RT for 5 min and diluted with dichloromethane (10 ml). After washing with water (20 ml), the dichloromethane solution was dried (Na₂SO₄) and concentrated in vacuo. The crude mesylate was mixed with naphthalen-2-ol (0.083 g, 0.58 mmol) and potassium carbonate (0.59 g, 4.27 mmol) in acetone (3 ml) and was heated at 50° C. overnight. Acetone was removed and water (50 ml) and ethyl acetate (40 ml) were added. After separation, the organic layer was washed with 1N sodium hydroxide (2×10 ml), brine (10 ml) and dried (Na₂SO₄). Concentration and flash column purification (SiO₂, 3% ethyl acetate-hexane) then afforded 1-benzyl-4-biphenyl-4-yl-3-(naphthalen-2-yloxymethyl) 1,2,3,6-tetrahydro-pyridine (0.131 g, 43%). ¹H—NMR (500 MHz, CDCl₃): δ 7.80-7.00 (m, 21H), 6.19 (m, 1H), 4.37 (t, 1H), 4.02 (dd, 1H), 3.73 (d, 1H), 3.64 (d, 1H), 3.44 (dd, 1H), 3.32 (d, 2H), 3.02 (d, 1H), 2.50 (m, 2H).

4-Biphenyl-4-yl-3-(naphthalen-2-ylmethoxy)-1,2,3,6-tetrahydro-pyridine (Compound 250):

1-Benzyl-4-biphenyl-4-yl-3-(naphthalen-2-yloxymethyl) 1,2,3,6-tetrahydro-pyridine (21 mg, 0.04 mmol) was mixed with 1-chloroethyl chloroformate (0.040 ml, 0.37 mmol) in dichloromethane and the resulting solution was stirred at 50° C. for 1 h. Evaporation under vacuum gave a residue, which was dissolved in methanol (3 ml) and was heated at 70° C. for 3 h. Methanol was removed and saturated aqueous sodium bicarbonate (30 ml) was added. Extraction with dichloromethane (3×20 ml), drying (Na₂SO₄) and concentration then gave a residue, which was purified by flash column (SiO₂, 3% methanol in dichloromethane) to produce 4-biphenyl-4-yl-3-(naphthalen-2-ylmethoxy)-1,2,3,6-tetrahydro-pyridine (16 mg, 94%). ¹H—NMR (500 MHz, CDCl₃): δ 7.76-7.05 (m, 16H), 6.23 (m, 1H), 4.20 (t, 1H), 4.03 (dd, 1H), 3.64-3.50 (m, 3H), 3.23 (m, 1H), 3.12 (dd, 1H). HPLC ret. Time: 6.87 min. LC—MS LC/MS:(ES⁺, Cacld for C₂₈H₂₅NO, 391.19), Found, M+1 392.16

### Example 14

Preparation of Compound 251

2,5-Dibromo-p-xylene (26.4 g, 0.1 mol) and NBS (39 g, 0.22 mol) were suspended in carbon tetrachloride (300 ml) and benzoyl peroxide (0.6 g) was added. A stream of nitrogen was bubbled through the reaction for 5 min. The reaction was heated with an oil bath of 100° C. for 2 h. Ethanol (200 ml) was added and the reaction was filtered. The remaining solid was washed with ethanol (50 ml) and dried under vacuum to obtain 1,4-dibromo-2,5-bis-bromomethyl-benzene as a white solid (13.36 g, 31.6%). ¹H—NMR (500 MHz, CDCl₃): δ 7.68 (s, 2H), 4.50 (s, 4H).

1,4-Dibromo-2,5-bis(2-trifluoromethylphenoxymethyl)benzene (49B):

A mixture of 1,4-dibromo-2,5-bis-bromomethyl-benzene (9.13 g, 21.6 mmol), 2-triflouromethyl-phenol (9 g, 55.5 mmol) and potassium carbonate (15 g, 108 mmol) in acetone (80 ml) was heated with an oil bath at 70° C. overnight. After cooling, acetone was removed and to the residue was added 2N sodium hydroxide (200 ml) ethyl ether (100 ml) and dichloromethane. The suspension was filtered and washed with water twice to give 1,4-dibromo-2,5-bis(2-trifluoromethylphenoxymethyl)benzene (9.66 g, 100%) as a white solid. ¹H—NMR (500 MHz, CDCl₃): δ 7.77 (s, 2H), 7.53 (d, 2H), 7.40 (t, 1H), 6.96-6.90 (m, 4H), 5.07 (s, 4H).

4′-(1,2,3,6-Tetrahydro-pyridin-4-yl)-2′,5′-bis-(2-trifluoromethyl-phenoxymethyl)-biphenyl-4-ol (Compound 251):

A mixture of 1,4-dibromo-2,5-bis(2-trifluoromethylphenoxymethyl)benzene (58.4 mg, 0.1 mmol), 4-(4,4,5,5-tetramethyl-[1,3,2]dioxaborolan-2-yl)-3,6-dihydro-2H-pyridine-1-carboxylic acid tert-butyl ester (30.9 mg, 0.1 mmol), 4-(4,4,5,5-tetramethyl-[1,3,2]dioxaborolan-2-yl)-phenol (22 mg, 0.1 mmol), 1,1′-bis (diphenylphosphino) ferrocene palladium (II) dichloride (7 mg) and potassium phosphate (127 mg, 0.6 mmol) in DME (1 ml) was heated at 70° C. overnight. Filtration through Celite, a wash with dichloromethane and concentration of the filtrates gave a residue, which was purified by flash chromatography (SiO₂, 5% to 50% ethyl acetate in hexane) to give the pure coupling product. Method A was used to generate the TFA salt of 4′-(1,2,3,6-tetrahydro-pyridin-4-yl)-2′,5′-bis-(2-trifluoromethyl-phenoxymethyl)-biphenyl-4-ol (9.4 mg). ¹H—NMR (500 MHz, methanol-d₄): δ 7.62-7.52 (m, 5H), 7.50 (t, 1H), 7.27 (d, 1H), 7.23 (d, 2H), 7.10 (t, 1H), 7.07 (t, 1H), 6.96 (d, 1H), 6.85 (d, 2H), 5.83 (br s, 1H), 5.25 (s, 2H), 5.12 (s, 2H), 3.82 (m, 2H), 3.45 (t, 2H), 2.72 (br s, 2H). HPLC ret. Time: 6.45 min. LC/MS:(ES⁺, Cacld for C₃₃H₂₇F₆NO₃ Exact Mass: 599.19), Found, 599.46.

### Example 15

Preparation of Compound 252

4′-(1,2,3,6-Tetrahydro-pyridin-4-yl)-2′,5′-bis-(2-trifluoromethyl-phenoxymethyl)-biphenyl-3-ol (Compound 252):

From 3-(4,4,5,5-tetramethyl-[1,3,2]dioxaborolan-2-yl)-phenol, following the same procedure as for the preparation of compound 251 and Method B, 4′-(1,2,3,6-tetrahydro-pyridin-4-yl)-2′,5′-bis-(2-trifluoromethyl-phenoxymethyl)-biphenyl-3-ol hydrochloride salt was obtained (13.8 mg). ¹H—NMR (500 MHz, methanol-d₄): δ 7.62-7.44 (m, 7H), 7.30 (d, 1H), 7.24 (t, 1H), 7.10 (t, 1H), 7.06 (t, 1H), 6.96 (d, 1H), 6.87-6.82 (m, 2H), 5.87 (br s, 1H), 5.28 (s, 2H), 5.14 (s, 2H), 3.83 (m, 2H), 3.50 (t, 2H), 2.72 (br s, 2H). HPLC ret. Time: 6.59 min. LC/MS:(ES⁺, Cacld for C₃₃H₂₇F₆NO₃ Exact Mass: 599.19), Found, M+1 600.20.

### Example 16

Preparation of Compound 253

4-[4-Furan-3-yl-2,5-bis-(2-trifluoromethyl-phenoxymethyl)-phenyl]-1,2,3,6-tetrahydro-pyridine (Compound 253):

From 3-furaneboronic acid, following the same procedure as for the preparation of compound 251 and Method B, 4-[4-furan-3-yl-2,5-bis-(2-trifluoromethyl-phenoxymethyl)-phenyl]-1,2,3,6-tetrahydro-pyridine hydrochloride salt was obtained (14.3 mg). ¹H—NMR (500 MHz, methanol-d₄): δ 7.73 (s, 1H), 7.67 (s, 1H), 7.63-7.55 (m, 6H) 7.36 (d, 1H), 7.21 (d, 1H), 7.10 (m, 2H), 6.71 (s, 1H), 5.89 (br s, 1H), 5.27 (s, 2H), 5.22 (s, 2H), 3.86 (m, 2H), 3.51 (t, 2H), 2.77 (br s, 2H). HPLC ret. Time: 7.04 min. LC/MS:(ES⁺, Cacld for C₃₁H₂₅F₆NO₃ Exact Mass: 573.17), Found, M+1 574.10.

### Example 17

Preparation of Compound 254

2-Bromo-1,3-bis(2-trifluoromethylphenoxymethyl)benzene (51B):

2-Bromo-1,3-bis-bromomethyl-benzene (0.1743 g, 0.73 mmol), 2-triflouromethylphenol (0.25 g, 1.54 mmol) and potassium carbonate (0.35 g, 2.53 mmol) were mixed in acetone (3 ml). After stirring at 50° C. overnight, the reaction was concentrated and water (30 ml) was added. Extraction with ethyl acetate (3×20 ml) and the combined organic phases were washed with 2 N NaOH (3×20 ml), brine and dried. Evaporation and washing with ether-hexane then gave 2-bromo-1,3-bis(2-trifluoromethylphenoxymethyl)benzene as a white solid (0.2201 g, 60%). ¹H—NMR (500 MHz, CDCl₃): δ 7.63 (t, 4H) 7.52 (t, 2H), 7.44 (t, 1H), 5.32 (s, 4H).

4-[2,6-Bis-(2-trifluoromethyl-phenoxymethyl)-phenyl]-1,2,3,6-tetrahydro-pyridine(Compound 254):

A mixture of 2-bromo-1,3-bis(2-trifluoromethylphenoxymethyl)benzene (0.137 g, 0.27 mmol), 4-(4,4,5,5-tetramethyl-[1,3,2]dioxaborolan-2-yl)-3,6-dihydro-2H-pyridine-1-carboxylic acid tert-butyl ester (0.0836 g, 0.27 mmol), 1,1′-bis (diphenylphosphino)ferrocene palladium (II) dichloride (0.020 g), potassium carbonate (0.112 g, 0.81 mmol) and potassium t-butoxide (0.078 g, 0.86 mmol) were mixed in DMF (3 ml) and heated at 80° C. for 2 days. The reaction was absorbed on silica and purified by two flash column (first with 3% to 20% ethyl acetate/hexane and 2^{nd} with dichloromethane) to give the boc compound, which, after Method A, was converted to the hydrochloride salt of 4-[2,6-bis-(2-trifluoromethyl-phenoxymethyl)-phenyl]-1,2,3,6-tetrahydro-pyridine. ¹H—NMR (500 MHz, methanol-d₄): δ 7.63-7.60 (M, 6H), 7.47 (t, 1H), 7.32 (d, 2H), 7.10 (t, 2H), 5.85 (s, 1H), 5.25 (d, 2H), 5.17 (d, 2H), 3.80 (m, 2H), 3.40 (t, 2H), 2.71 (br s, 2H). HPLC ret. Time: 7.09 min. LC/MS:(ES⁺, Cacld for C₂₇H₂₃F₆NO₂, Exact Mass: 507.16, Found, M+1 508.0.

### Example 18

Preparation of Compound 256

1,4-Bis-bromomethyl-2-iodo-benzene (52B):

Iodo-p-xylene (25.01 g, 0.108 mol), NBS (40.3 g, 0.226 mol) and benzoyl peroxide (2 g) were mixed in carbon tetrachloride (250 ml). After refluxing for 4 h, more NBS (6 g) and benzoyl peroxide (0.6 g) were added and the mixture was refluxed overnight. Cooling to RT, filtration and concentration of the filtrate gave a solid, which was recrystalized from hexane to give 1,4-bis-bromomethyl-2-iodo-benzene as white crystals (7.02 g, 17%). ¹H—NMR (500 MHz, CDCl₃): δ 7.90 (s, 1H), 7.47 (d, 2H), 7.38 (d, 1H), 4.70 (s, 2H), 4.37 (s, 2H).

1,4-Bis(2-trifluoromethylphenoxymethyl)-2-iodo-benzene (53B):

1,4-Bis(2-trifluoromethylphenoxymethyl)-2-iodo-benzene was prepared following the same procedure as for compound 254 in 84% yield as a white solid. ¹H—NMR (500 MHz, CDCl₃): δ 7.98 (s, 1H), 7.65-7.46 (m, 6H), 7.07-7.00 (m, 4H), 5.17 S, 2H), 5.14 (s, 2H).

4-[2,5-Bis-(2-trifluoromethyl-phenoxymethyl)-phenyl]-3,6-dihydro-2H-pyridine-1-carboxylic Acid tert-butyl Ester (54B):

A mixture of 1,4-Bis(2-trifluoromethylphenoxymethyl)-2-iodo-benzene (0.200 g, 0.36 mmol), 4-(4,4,5,5-tetra-methyl-[1,3,2]dioxaborolan-2-yl)-3,6-dihydro-2H-pyridine-1-carboxylic acid tert-butyl ester (0.112 g, 0.36 mmol), 1,1′-bis (diphenylphosphino)ferrocene palladium (II) dichloride (0.030 g)and potassium phosphate (0.230 g, 1.08 mmol) were mixed in DME and heated at 70° C. for 2 days. The reaction was filtered through Celite and the filtrates were concentrated to give the crude product, which was purified by flash chromatography (SiO₂, 5% to 15% ethyl acetate in hexane) to generate 4-[2,5-bis-(2-trifluoromethyl-phenoxymethyl)-phenyl]-3,6-dihydro-2H-pyridine-1-carboxylic acid tert-butyl ester (0.1886 g, 71%).

4-[2,5-Bis-(2-trifluoromethyl-phenoxymethyl)-phenyl]-1,2,3,6-tetrahydro-pyridine (Compound 256):

Following Method B, the HCl salt of 4-[2,5-bis-(2-trifluoromethyl-phenoxymethyl)-phenyl]-1,2,3,6-tetrahydro-pyridine was obtained from 4-[2,5-bis-(2-trifluoromethyl-phenoxymethyl)-phenyl]-3,6-dihydro-2H-pyridine-1-carboxylic acid tert-butyl ester. ¹H—NMR (500 MHz, methanol-d₄): δ 7.63-7.53 (m, 5H). 7.47 (d, 1H), 7.40 (s, 1H), 7.30 (d, 1H), 7.25 (d, 1H), 7.10 (d, 1H), 7.08 (d, 1H), 5.82 (br s, 1H), 5.26 (s, 2H), 5.17 (s, 2H), 3.82 (br s, 2H), 3.46 (t, 2H), 2.72 (br s, 2H), HPLC ret. Time: 6.90 min. LC/MS: (ES⁺, Cacld for C₂₇H₂₃F₆NO2, Exact Mass: 507.16, Found, M+1 508.11.

### Example 19

Preparation of Compound 257

2-Bromo-4-methyl-benzoic Acid Methyl Ester (55B):

2-Bromo-4-methylbenzoic acid (24.92 g, 0.116 mol) was mixed with methanol (200 ml) and concentrated sulfuric acid (10 ml). After refluxing for 2 days, the mixture was cooled to RT and methanol was removed under vacuum. The rest was taken in ethyl acetate (300 ml) and washed with water, brine and dried. Evaporation then gave 2-bromo-4-methyl-benzoic acid methyl ester (25.86 g, 97%) as a white solid. ¹H—NMR (500Mz, CDCl₃) δ 7.77 (d, 1H) 7.53 (s, 1H), 7.28 (d, 1H), 3.97 (s, 3H), 2.50 (s, 3H).

2-Bromo-4-bromomethyl-benzoic Acid Methyl Ester (56B):

The 2-bromo-4-methyl-benzoic acid methyl ester (11.05 g, 48 mmol), NBS (10.30 g, 58 mmol) and benzoyl peroxide (0.6 g) were mixed in benzene (200 ml) and the resulting mixture was refluxed for 2 h. The reaction was absorbed on silica gel and applied to a flash column (SiO2, 4% to 10% ethyl acetate in hexane). The first fraction was the starting material (7.313 g, 66%) and the polar fraction was the desired 2-bromo-4-bromomethyl-benzoic acid methyl ester (5.51 g, 37%) as a white solid. ¹H—NMR (500 MHz, CDCl₃): δ 7.81 (d, 1H), 7.71 (s, 1H), 7.40 (d, 1H), 4.42 (s, 2H), 3.95 (s, 3H)

2-Bromo-4-(2-trifluoromethyl-phenoxymethyl)-benzoic Acid Methyl Ester (57B):

2-Bromo-4-(2-trifluoromethyl-phenoxymethyl)-benzoic acid methyl ester was prepared starting with 2-bromo-4-bromomethyl-benzoic acid methyl ester, 2-trifluoromethylphenol (1.3 eq) and potassium carbonate (3 eq) by following the same procedure as described for compound 51B. ¹H—NMR (500 MHz, CDCl₃): δ 7.86 (d, 1H), 7.77 (s, 1H), 7.63 (d, 1H), 7.52-7.47 (m, 2H), 7.08 (t, 1H), 7.00 (d, 1H), 5.20 (s, 2H), 3.96 (s, 3H).

4-[2-Methoxycarbonyl-5-(2-trifluoromethyl-phenoxymethyl)-phenyl]-3,6-dihydro-2H-pyridine-1-carboxy

lic acid tert-butyl ester (58B):

By the same procedure as described for compound 51B, 4-[2-methoxycarbonyl-5-(2-trifluoromethyl-phenoxymethyl)-phenyl]-3,6-dihydro-2H-pyridine-1-carboxylic acid tert-butyl ester was obtained from 2-bromo-4-(2-trifluoromethyl-phenoxymethyl)-benzoic acid methyl ester. ¹H—NMR (500Mz, CDCl₃): δ 7.89 (d, 2H), 7.62 (d, 1H), 7.50 (t, 1H), 7.43 (t, 1H), 7.27 (s, 1H), 7.06 (t, 1H), 7.02 (d, 1H), 5.55 (br s, 1H), 5.25 (s, 2H), 4.06 (br s, 2H), 3.86 (s, 3H), 3.66 (m, 2H), 2.34 (br s, 2H), 1.47 (s, 9H).

4-[2-Hydroxymethyl-5-(2-trifluoromethyl-phenoxymethyl)-phenyl]-3,6-dihydro-2H-pyridine-1-carboxyli

c Acid tert-butyl Ester (59B):

To a solution of 4-[2-methoxycarbonyl-5-(2-trifluoromethyl-phenoxymethyl)-phenyl]-3,6-dihydro-2H-pyridine-1-carboxylic acid tert-butyl ester (0.985 g, 2 mmol) in THF (10 ml) at −78° C. was added dropwise a 1M DIBAL-hexane solution (6 ml, 6 mmol). After 30 min, the reaction was brought to RT for 1 h, then poured onto a saturated potassium sodium tartrate solution. Separation, extraction with dichloromethane (2×50 ml), washing with brine, drying and evaporation then gave 4-[2-hydroxymethyl-5-(2-trifluoromethyl-phenoxymethyl)-phenyl]-3,6-dihydro-2H-pyridine-1-carboxylic acid tert-butyl ester as a white solid (0.8518 g, 91.7%).

4-[2-Chloromethyl-5-(2-trifluoromethyl-phenoxymethyl)-phenyl]-3,6-dihydro-2H-pyridine-1-carboxylic Acid tert-butyl Ester (60B):

To 4-[2-hydroxymethyl-5-(2-trifluoromethyl-phenoxymethyl)-phenyl]-3,6-dihydro-2H-pyridine-1-carboxylic acid tert-butyl ester (0.315 g, 0.68 mmol) in dichloromethane (5 ml) at 0° C. was added pyridine (0.083 ml, 1 mmol) and methanesulfonyl chloride (0.079 ml, 1.1 mmol). After 1 h, the same amounts of pyridine and mesyl chloride were added. Triethylamine (0.24 ml) was added and the reaction was stirred at RT for 5 min, diluted with ethyl acetate (60 ml) and ether (20 ml). Washing with cold 1 M HCl (2×), water, saturated sodium bicarbonate, brine, drying and concentration under vacuum gave the crude product, which was purified by flash column (SiO2, 20% ethyl acetate in hexane) to give 4-[2-chloromethyl-5-(2-trifluoromethyl-phenoxymethyl)-phenyl]-3,6-dihydro-2H-pyridine-1-carboxylic acid tert-butyl ester (0.297 g, 90.7%).

4-[2-(Biphenyl-4-yloxymethyl)-5-(2-trifluoromethyl-phenoxymethyl)-phenyl]-3,6-dihydro-2H-pyridine-1-carboxylic Acid tert-butyl Ester (61B):

A mixture of 4-[2-chloromethyl-5-(2-trifluoromethyl-phenoxymethyl)-phenyl]-3,6-dihydro-2H-pyridine-1-carboxylic acid tert-butyl ester (0.012 mg, 0.025 mmol), 4-phenylphenol (0.028 g, 0.16 mmol) and potassium carbonate (0.050 g, 0.36 mmol) in acetone (1 ml) was heated at 60° C. overnight. Following the same procedure as for compound 51B. The reaction was diluted with ether (30 ml), washed with 1 N sodium hydroxide (2×20 ml), dried and evaporated. Pure 4-[2-(biphenyl-4-yloxymethyl)-5-(2-trifluoromethyl-phenoxymethyl)-phenyl]-3,6-dihydro-2H-pyridine-1-carboxylic acid tert-butyl ester was then obtained by flash column (SiO2, 5% to 15% ethyl acetate in hexane).

4-[2-(Biphenyl-4-yloxymethyl)-5-(2-trifluoromethyl-phenoxymethyl)-phenyl]-1,2,3,6-tetrahydro-pyridine (Compound 257):

The HCl salt of 4-[2-(biphenyl-4-yloxymethyl)-5-(2-trifluoromethyl-phenoxymethyl)-phenyl]-1,2,3,6-tetrahydro-pyridine was obtained from 4-[2-(biphenyl-4-yloxymethyl)-5-(2-trifluoromethyl-phenoxymethyl)-phenyl]-3,6-dihydro-2H-pyridine-1-carboxylic acid tert-butyl ester by Method B. ¹H—NMR (500 MHz, methanol-d₄): δ 7.66-7.10 (m, 16H), 5.80 (br s, 1H), 5.27 (s, 2H), 5.13 (s, 2H), 3.83 (br s, 2H), 3.43 (t, 2H), 2.72 (br s, 2H). HPLC ret. Time: 7.26 min. LC/MS: (ES⁺, Cacld for C₃₂H₂₈F₃NO₂ Exact Mass: 515.21), Found, M+1 516.22.

### Example 20

Preparation of Compound 258

Naphthalene-1-carboxylic Acid 2-(1,2,3,6-tetrahydro-pyridin-4-yl)-4-(2-trifluoromethyl-phenoxymethyl )-benzyl ester (Compound 258):

To a solution 4-[2-hydroxymethyl-5-(2-trifluoromethyl-phenoxymethyl)-phenyl]-3,6-dihydro-2H-pyridine-1-carboxylic acid tert-butyl ester (0.015 mg, 0.032 mmol) in dichloromethane (1 ml) was added pyridine (0.012 ml, 0.15 mmol) and naphthalene-1-carbonyl chloride (0.017 mg, 0.09 mmol). After 2 days, the reaction was diluted with ethyl acetate (20 ml), washed with cold 1 N HCl (2×), water, saturated sodium bicarbonate, brine and dried. Evaporation and flash column purification then gave pure N-boc intermediate, which was converted to the HCl salt of naphthalene-1-carboxylic acid 2-(1,2,3,6-tetrahydro-pyridin-4-yl)-4-(2-trifluoromethyl-phenoxymethyl )-benzyl ester by Method B (0.008 mg, 45%). ¹H—NMR (500 MHz, methanol-d₄): δ 8.90 (d, 1H), 8.21 (d, 1H), 8.11 (d, 1H), 7.97 (d, 1H), 7.67 (d, 1H), 7.63-7.47 (m, 6H), 7.40 (s, 1H), 7.25 (d, 1H), 7.06 (t, 1H), 5.78 (br s, 1H), 5.50 (s, 2H), 5.30 (s, 2H), 3.85 (br s, 2H), 3.44 (t, 2H), 2.71 (br s, 2H). HPLC ret. Time: 7.02 min. LC/MS:(ES⁺, Cacld for C₃₁H₂₆F₃NO3, Exact Mass: 517.19), Found, M+1 518.10.

### Example 21

Preparation of Compound 259

Carbonic Acid Naphthalen-1-yl Ester 2-(1,2,3,6-tetrahydro-pyridin-4-yl)-4-(2-trifluoromethyl-phenoxy methyl)-benzyl Ester (Compound 259):

Following the same procedure as for the preparation of compound 258 and Method A. the TFA salt of carbonic acid naphthalen-1-yl ester 2-(1,2,3,6-tetrahydro-pyridin-4-yl)-4-(2-trifluoromethyl-phenoxymethyl)-benzyl ester was prepared. ¹H—NMR (500 MHz, methanol-d₄): δ 8.17-6.77 (m, 14H), 5.71 (br s, 1H), 5.40 (s, 2H), 5.28 (s, 2H), 3.82 (br s, 2H), 3.45 (t, 2H), 2.64 (br s, 2H). HPLC ret. Time: 6.96 min. LC/MS: (ES⁺, Cacld for C₃₁H₂₆F₃NO₄ Exact Mass: 533.18), Found, M+1 534.10.

### Example 22

Preparation of Compound 260

5-[2-(1,2,3,6-Tetrahydro-pyridin-4-yl)-4-(2-trifluoromethyl-phenoxymethyl)-benzyloxy]-quinoline (62B):

Following the same procedure as for the preparation of compound 257 and Method B, the TFA salt of 5-[2-(1,2,3,6-tetrahydro-pyridin-4-yl)-4-(2-trifluoromethylphenoxymethyl)-benzyloxy]-quinoline was prepared. ¹H—NMR (500 MHz, methanol-d₄): δ 9.28 (d, 1H), 9.13 (dd, 1H), 8.06 (t, 1H), 7.92 (dd, 1H), 7.78 (d, 1H), 7.71 (d, 1H), 7.62-7.53 (m, 3H), 7.47-7.45 (m, 2H), 7.26 (d, 1H), 7.09 (t, 1H), 5.82 (br s, 1H), 5.41 (s, 2H), 5.28 (s, 2H), 3.77 (br s, 2H), 3.42 (t, 2H), 2.72 (br s, 2H). HPLC ret. time: 5.56 min. LC/MS: (ES⁺, Cacld C₂₉H₂₅F₃N₂O₂ Exact Mass: 490.19), Found, M+1 491.13.

### Example 23

Preparation of Compound 261

4-(4-Bromo-2-methoxycarbonyl-phenyl)-3,6-dihydro-2H-pyridine-1-carboxylic acid tert-butyl ester (64B):

5-Bromo-2-iodo-benzoic acid methyl ester (63B) was prepared following the same method as described for compound 55B. 4-(4-bromo-2-methoxycarbonyl-phenyl)-3,6-dihydro-2H-pyridine-1-carboxylic acid tert-butyl ester (64B) was prepared according to the same procedure as described for compound 51B, ¹H—NMR (500 MHz, CDCl₃): δ 7.82 (s, 1H), 7.43 (d, 1H), 6.93 (d, 1H), 5.36 (br s, 1H), 3.90 (br s, 3H), 3.70 (s, 3H), 3.47 (br s, 2H), 2.15 (br s, 2H), 1.38 (s, 9H).

4-[4-Bromo-2-(2-trifluoromethyl-phenoxymethyl)-phenyl]-3,6-dihydro-2H-pyridine-1-carboxylic acid tert-butyl ester (67B):

4-(4-Bromo-2-hydroxymethyl-phenyl)-3,6-dihydro-2H-pyridine-1-carboxylic acid tert-butyl Ester (65B)was prepared following the same procedure as for compound 59B. 4-(4-Bromo-2-chloromethyl-phenyl)-3,6-dihydro-2H-pyridine-1-carboxylic acid tert-butyl ester (66B) was prepared following the same procedure as for compound 60B. 4-[4-Bromo-2-(2-trifluoromethyl-phenoxymethyl)-phenyl]-3,6-dihydro-2H-pyridine-1-carboxylic acid tert-butyl ester (67B) was prepared according to the method described for compound 61B.

4-[2′-Hydroxymethyl-3-(2-trifluoromethyl-phenoxymethyl)-biphenyl-4-yl]-3,6-dihydro-2H-pyridine-1-c

arboxylic Acid tert-butyl Ester (68B):

A mixture of 4-[4-bromo-2-(2-trifluoromethyl-phenoxymethyl)-phenyl]-3,6-dihydro-2H-pyridine-1-carboxylic acid tert-butyl ester (0.20 g, 0.39 mmol), 2-hydroxymethylphenyl boronic acid (0.078 g, 0.58 mmol), potassium phosphate (0.248 g, 1.2 mmol) and 1,1′-bis (diphenylphosphino) ferrocene palladium (II) dichloride (0.025 g) in DME (2 ml) was heated at 70° C. for 2 days. Filtrations through Celite, concentration, and flash column purification (SiO₂, 20 to 30% ethyl acetate in hexane) generated 4-[2′-hydroxymethyl-3-(2-trifluoromethyl-phenoxymethyl)-biphenyl-4-yl]-3,6-dihydro-2H-pyridine-1-carboxylic acid tert-butyl ester.

2-Trifluoromethyl-benzoic acid 4′-(1,2,3,6-tetrahydro-pyridin-4-yl)-3′-(2-trifluoromethyl-phenoxymet hyl)-biphenyl-2-ylmethyl ester (Compound 261):

The TFA salt of 2-trifluoromethyl-benzoic acid 4′-(1,2,3,6-tetrahydro-pyridin-4-yl)-3′-(2-trifluoromethyl-phenoxymethyl)-biphenyl-2-ylmethyl ester was prepared from 4-[2′-hydroxymethyl-3-(2-trifluoromethyl-phenoxymethyl)-biphenyl-4-yl]-3,6-dihydro-2H-pyridine-1-carboxylic acid tert-butyl ester following the same procedure as described for compound 258 and Method A. ¹H—NMR (500 MHz, CDCl₃) δ 7.71 (d, 1H), 7.69 (d, 1H), 7.61-7.34 (m, 10H), 7.25 (d, 1H), 7.12 (d, 1H), 7.01 (t, 1H), 5.79 (br s, 1H), 5.32 (s, 2H), 5.08 (s, 2H), 3.80 (br s, 2H), 3.43 (br s, 2H), 2.74 (br s, 2H). HPLC ret. time: 6.96 min. LC/MS: (ES⁺, Cacld for C₃₄H₂₇F₆NO3, Exact Mass: 611.19), Found, M+1 612.20.

### Example 24

Preparation of Compound 262

4-[3-(2-Trifluoromethyl-phenoxymethyl)-biphenyl-4-yl]-1,2,3,6-tetrahydro-pyridine (Compound 262):

The HCl salt of 4-[3-(2-trifluoromethyl-phenoxymethyl)-biphenyl-4-yl]-1,2,3,6-tetrahydro-pyridine was prepared following the same procedure as described for compound 68B and Method B. ¹H—NMR (500 MHz, methanol-d₄): δ 7.88 (d, 1H), 7.66-7.58 (m, 5H), 7.44 (t, 2H), 7.36 (d, 2H), 7.32 (d, 1H), 7.10 (t, 1H), 5.82 (br s, 1H), 5.27 (s, 2H), 3.83 (br s, 2H), 3.47 (t, 2H), 2.72 (br s, 2H). HPLC ret. time: 6.54 min. LC/MS: (ES⁺, Cacld for C₂₅H₂₂F₃NO, Exact Mass: 409.17), Found, M+1 410.20.

### Example 25

Preparation of Compound 263

5-Amino-2-bromo-4-methyl-benzoic acid methyl ester (69B):

Following a similar procedure reported in J. Med. Chem. 1999, 42, 3701, 5-amino-2-bromo-4-methyl-benzoic acid methyl ester was prepared from methyl 3-amino-4-methylbenzoate in 77% yield. ¹H—NMR (500 MHz, CDCl₃).7.34 (s, 1H), 7.16 (s, 1H), 3.90 (s, 3H), 3.74 (br s, 2H), 2.19 (s, 3H).

2-Bromo-5-iodo-4-methyl-benzoic Acid Methyl Ester (70B):

To a solution of 5-amino-2-bromo-4-methyl-benzoic acid methyl ester (2.43 g, 10 mmol) in 3N hydrochloric acid and acetone (210 ml) at −5C was added sodium nitrite (0.76 g, 11 mmol) in water (11 ml). After 30 min, potassium iodide (2.89 g, 17 mmol) was added and the resulting reaction was stirred at RT overnight. After adding sodium sulfite (5 g), the reaction was concentrated and extracted with dichloromethane (3×60 ml). Flash chromatography (SiO₂, dichloromethane) then gave 2-bromo-5-iodo-4-methyl-benzoic acid methyl ester (2.65 g, 75%). ¹H—NMR (500 MHz, CDCl₃) 8.28 (s, 1H), 7.55 (s, 1H), 3.94 (s, 3H), 2.50 (s, 3H).

2-Bromo-5-iodo-4-(2-trifluoromethyl-phenoxymethyl)-benzoic Acid Methyl Ester (71B):

A mixture of 2-bromo-5-iodo-4-methyl-benzoic acid methyl ester (1.0249 g, 2.89 mmol), NBS (0.617 g, 3.48 mmol) and benzoyl peroxide (0.04 g) in carbon tetrachloride (5 ml) was heated at 100° C. for 6 h, during which time a solution of additional benzoyl peroxide (0.06 g) in carbon tetrachloride (1 ml) was added through a syringe from time to time. The mixture was absorbed on silica and was applied on a flash column (SiO₂, dichloromethane). The crude product thus obtained was combined with 2-triflouromethylphenol and potassium carbonate (1 g) in acetone (6 ml). Work-up as described for compound 61B and column purification (SiO2, 2.5% to 5% ethyl acetate in hexane) gave recovered 70B (0.2929 g, 29%) and 2-bromo-5-iodo-4-(2-trifluoromethyl-phenoxymethyl)-benzoic acid methyl ester (0.8921 g, 60%).

4-[4-Bromo-5-methoxycarbonyl-2-(2-trifluoromethyl-phenoxymethyl)-phenyl]-3,6-dihydro-2H-pyridine-1-carboxylic Acid tert-butyl Ester (72B):

Following the same procedure as for compound 58B, 4-[4-bromo-5-methoxycarbonyl-2-(2-trifluoromethyl-phenoxymethyl)-phenyl]-3,6-dihydro-2H-pyridine-1-carboxylic acid tert-butyl ester was prepared from 2-bromo-5-iodo-4-(2-trifluoromethyl-phenoxymethyl)-benzoic acid methyl ester.

4-[4-Furan-3-yl-5-methoxycarbonyl-2-(2-trifluoromethyl-phenoxymethyl)-phenyl]-3,6-dihydro-2H-pyrid

ine-1-carboxylic Acid tert-butyl Ester (73B):

A mixture of 4-[4-bromo-5-methoxycarbonyl-2-(2-trifluoromethyl-phenoxymethyl)-phenyl]-3,6-dihydro-2H-pyridine-1-carboxylic acid tert-butyl ester (0.298 g, 0.52 mmol), 3-furanylboronic acid (0.100 g, 0.89 mmol), potassium phosphate (0.432 g, 2.0 mmol) and 1,1′-bis (diphenylphosphino)ferrocene palladium (II) dichloride (0.05 g) in DME (4 ml) was heated at 70° C. overnight. Filtration though Celite, concentration and purification by flash column (SiO2, 15 to 20% ethyl acetate in hexane) gave 4-[4-furan-3-yl-5-methoxycarbonyl-2-(2-trifluoromethyl-phenoxymethyl)-phenyl]-3,6-dihydro-2H-pyridine-1-carboxylic acid tert-butyl ester (0.2379 g, 82%).

4-[4-Furan-3-yl-5-hydroxymethyl-2-(2-trifluoromethyl-phenoxymethyl)-phenyl]-3,6-dihydro-2H-pyridin

e-1-carboxylic Acid tert-butyl Ester (74B):

The DIBAL reduction of 4-[4-furan-3-yl-5-methoxycarbonyl-2-(2-trifluoromethyl-phenoxymethyl)-phenyl]-3,6-dihydro-2H-pyridine-1-carboxylic acid tert-butyl ester was conducted using the same procedure as for compound 59B to afford 4-[4-furan-3-yl-5-hydroxymethyl-2-(2-trifluoromethyl-phenoxymethyl)-phenyl]-3,6-dihydro-2H-pyridine-1-carboxylic acid tert-butyl ester.

Isonicotinic acid 2-furan-3-yl-5-(1,2,3,6-tetrahydro-pyridin-4-yl)-4-(2-trifluoromethyl-phenoxymethy

l)-benzyl Ester (Compound 263):

The TFA salt of isonicotinic acid 2-furan-3-yl-5-(1,2,3,6-tetrahydro-pyridin-4-yl)-4-(2-trifluoromethyl-phenoxymethyl)-benzyl ester was prepared following the same procedure as described for compound 258 and Method A. ¹H—NMR (500 MHz, methanol-d₄): δ 7.88 (d, 1H), 7.66-7.58 (m, 5H), 7.44 (t, 2H), 7.36 (d, 2H), 7.32 (d, 1H), 7.10 (t, 1H), 5.82 (br s, 1H), 5.27 (s, 2H), 3.83 (br s, 2H), 3.47 (t, 2H), 2.72 (br s, 2H). HPLC ret. time: 5.93 min. LC/MS: (ES⁺, Cacld for C₃₀H₂₅F₃N₂O₄ Exact Mass: 534.18), Found, M+1 535.20.

### Example 26

Preparation of Compound 264

1,4,5,6-Tetrahydro-2H-azepino[4,5-b]indole-3,5-dicarboxylic acid 3-tert-butyl Ester 5-methyl Ester (75B):

1,2,3,4,5,6-Hexahydro-azepino[4,5-b]indole-5-carboxylic acid methyl ester (0.100 g, 0.4 mmol) and di-t-butyl dicarbonate (0.164 g, 0.75 mmol) were mixed in methanol (3 ml) and triethylamine (0.12 ml, 0.86 mmol) was added. After stirring at RT overnight, the reaction was concentrated and purified by flash column (SiO₂, 20% ethyl acetate-hexane) to give 1,4,5,6-tetrahydro-2H-azepino[4,5-b]indole-3,5-dicarboxylic acid 3-tert-butyl ester 5-methyl ester (0.129 g, 91%).

1,4,5,6-Tetrahydro-2H-azepino[4,5-b]indole-3,5-dicarboxylic Acid 3-tert-butyl Ester (76B):

1,4,5,6-Tetrahydro-2H-azepino[4,5-b]indole-3,5-dicarboxylic acid 3-tert-butyl ester 5-methyl ester (0.100 g, 0.29 mmol)was mixed with ethanol(3 ml) and 2N NaOH (2 ml). After stirring at 50° C. for 45 min, the reaction evaporated and the residue acidified with cold dilute HCl to pH 2. Extraction with ethyl acetate (2×20 ml), washing with brine, drying and concentration produced crude 1,4,5,6-tetrahydro-2H-azepino[4,5-b]indole-3,5-dicarboxylic acid 3-tert-butyl ester (0.097 mg), which was pure enough for the next step.

1,2,3,4,5,6-Hexahydro-azepino[4,5-b]indole-5-carboxylic Acid naphthalen-2-ylamide (Compound 264):

A mixture of 1,4,5,6-tetrahydro-2H-azepino[4,5-b]indole-3,5-dicarboxylic acid 3-tert-butyl ester (10 mg, 0.03 mmol), 2-aminonaphthalene (6 mg, 0.04 mmol) EDC (11.6 mg, 0.06 mmol), HOBt (8.2 mg, 0.06 mmol) and triethyamine (0.021 mL, 2.0 mmol) in dichloromethane (1 ml) was stirred at RT for 24 h. The reaction was diluted with ethyl acetate (20 ml) and washed with cold 1 N HCl and brine. Drying, evaporation, and purification by flash column (20% ethyl acetate in hexane) gave the boc intermediate, which, upon treatment by Method A, was converted to TFA salt of 1,2,3,4,5,6-hexahydro-azepino[4,5-b]indole-5-carboxylic acid naphthalen-2-ylamide (7.5 mg, 52% from 47). ¹H—NMR (500 MHz, methanol-d₄): δ 8.40 (d, 2H), 8.32 (s, 1H), 8.03-7.94 (m, 3H) 7.55-7.44 (m, 5H), 7.16 (t, 1H), 7.07 (t, 1H), 4.42 br s, 1H), 4.09 (dd, 1H), 3.85 (m, 1H), 3.70 (d, 1H), 3.40-3.30 (m, 3H). HPLC ret. time: 6.06 min. LC/MS:(ES⁺, Cacld for C₂₃H₂₁N₃O Exact Mass: 355.17), Found, M+1 356.20.

### Example 27

Preparation of Compound 265

2-(Naphthalen-2-ylcarbamoyl)-piperazine-1,4-dicarboxylic acid 1-benzyl Ester 4-tert-butyl Ester (77B):

2-(Naphthalen-2-ylcarbamoyl)-piperazine-1,4-dicarboxylic acid 1-benzyl ester 4-tert-butyl ester was prepared according to the same procedure as described for compound 264. 2-(Naphthalen-2-ylcarbamoyl)-piperazine-1,4 dicarboxylic acid 1-benzyl ester 4-tert-butyl ester (0.36 g) was hydrogenated with 10% Pd/C in methanol using a hydrogen balloon for 3 h. Filtration, concentration and column purification (SiO₂, 1:1 ether/hexane) then gave 3-(naphthalen-2-ylcarbamoyl)-piperazine-1-carboxylic acid tert-butyl ester (0.118 g). ¹H—NMR (500 MHz, CDCl₃) δ 9.00 (s, 1H), 8.07 (s, 1H), 7.78-7.74 (m, 3H), 7.50-7.38 (m, 3H), 4.17-2.36 (m, 8H), 1.47 (s, 9H).

1-[3-(2-Trifluoromethyl-phenoxymethyl)-benzoyl]-piperazine-2-carboxylic Acid naphthalen-2-ylamide (Compound 265):

A mixture of 3-(Naphthalen-2-ylcarbamoyl)-piperazine-1-carboxylic acid tert-butyl ester (0.0168 g, 0.05 mmol), pyridine (0.008 ml, 0.098 mmol), 3-chloromethylbenzoyl chloride (0.010 ml, 0.07 mmol) in dichloromethane (1 ml) was stirred for 5 min. The reaction was diluted with ethyl acetate (15 ml), washed with cold 1 N HCl (2×10 ml), 1N NaOH (2×10 ml), brine, and dried (Na₂SO₄). Evaporation of the solvents gave crude 4-(3-chloromethyl-benzoyl)-3-(naphthalen-2-ylcarbamoyl)-piperazine-1-carboxylic acid tert-butyl ester, which was mixed with 2-trifluoromethylphenol (0.076 g, 0.47 mmol) and potassium carbonate (0.15 g. 1.1 mmol) in acetone (3 ml). After stirring at 50° C. overnight and the same work-up as for compound 61B the Boc intermediate was obtained, which after Method B treatment, was converted into the HCl salt. Preparative HPLC then generated the TFA salt of 1-[3-(2-trifluoromethyl-phenoxymethyl)-benzoyl]-piperazine-2-carboxylic acid naphthalen-2-ylamide. ¹H—NMR (500 MHz, methanol-d₄): δ 8.28 (s, 1H), 7.85-7.78 (m, 3H) 7.68-7.06 (m, 12H), 5.28 (s, 2H), 3.98 (d, 1H), 3.82 (t, 1H), 3.52 (d, 1H), 3.49 (d, 1H), 3.35-3.22 (m, 3H). HPLC ret. time: 6.45 min. LC/MS: (ES⁺, Cacld for C₃₀H₂₆F₃N₃O₃ Exact Mass: 533.19), Found, M 534.3.

### Example 28

Preparation of Compound 266 6-Phenyl-2-pyridin-4-yl-pyrimidin-4-ol (78B):

A mixture of 4-amidinopyridine hydrochloride (1.57 g, 10 mmol) and 3-oxo-3-phenyl-propionic acid ethyl ester (3.0 g, 15.6 mmol) was refluxed in ethanol overnight. Cooling to RT, filtration and washing with ethanol then gave 6-phenyl-2-pyridin-4-yl-pyrimidin-4-ol as a solid (1.8059 g, 73%). 2-(1-Benzyl-1,2,3,6-tetrahydro-pyridin-4-yl)-6-phenyl-pyrimidin-4-ol (79B):

A solution of 6-phenyl-2-pyridin-4-yl-pyrimidin-4-ol (0.43 g, 1.7 mmol) and benzyl bromide (0.32 g, 1.9 mmol) in chloroform (8 ml) and methanol (2 ml) was heated at 65° C. overnight. After removal of the solvents in vacuo, the residue was diluted with methanol (10 ml) and water (5 ml). Sodium borohydride (0.26 g, 6.8 mmol) was added by parts. Water (50 ml) was added and the resulting solution was extracted with dichloromethane (3×50 ml) and the combined organic phases were concentrated and the resulting solid was washed with water and methanol. Pure 2-(1-benzyl-1,2,3,6-tetrahydro-pyridin-4-yl)-6-phenyl-pyrimidin-4-ol was obtained as a solid (0.43 g, 74%).

4-(4-Hydroxy-6-phenyl-pyrimidin-2-yl)-piperidine-1-carboxylic Acid tert-butyl Ester (80B):

To a solution of 2-(1-benzyl-1,2,3,6-tetrahydro-pyridin-4-yl)-6-phenyl-pyrimidin-4-ol (0.185 g, 0.54 mmol) and di-tert-butyl dicarbonate (0.19 ml, 0.79 mmol) in methanol (5 ml) and ethyl acetate (3 ml) was added 10% Pd/C (30 mg). The resulting mixture was hydrogenated under a H₂ balloon overnight. The reaction was filtered through Celite and the filtrates were concentrated to afford a residue as the crude 4-(4-hydroxy-6-phenyl-pyrimidin-2-yl) -piperidine-1-carboxylic acid tert-butyl ester, which was used directly for the next step without further purification.

6-Phenyl-2-piperidin-4-yl-3-(2-trifluoromethyl-benzyl)-3H-pyrimidin-4-one (Compound 266):

Crude 4-(4-hydroxy-6-phenyl-pyrimidin-2-yl)-piperidine-1-carboxylic Acid tert-butyl Ester (0.026 g, 0.07 mmol) was mixed with 2-trifluoromethylbenzyl bromide (0.0875 g, 0.37 mmol) and potassium carbonate (0.105 9, 0.75 mmol) in acetone (1 ml). After stirring at 50° C. overnight, the reaction was cooled to RT, diluted with ethyl acetate (20 ml) and washed with brine. Drying (Na₂SO₄) and concentration gave a residue, which was purified by flash column (SiO₂, 5% to 10% ethyl acetate in hexane) to generate the N-alkylated boc intermediate, which, after treatment of Method B, was converted to the HCl salt of 6-phenyl-2-piperidin-4-yl-3-(2-trifluoromethyl-benzyl)-3H-pyrimidin-4-one. ¹H—NMR (500 MHz, methanol-d₄): δ 8.03 (d, 2H), 7.85-7.59 (m, 7H), 7.58 (s, 1H), 5.90 (s, 2H), 3.61-3.56 (m, 3H), 3.28-3.22 (m, 2H), 2.39-2.24 (m, 4H). HPLC ret. time: 6.45 min. LC/MS: (ES⁺, Cacld, for C₂₃H₂₂F₃N₃O, Exact Mass: 413.17), Found, M+1 414.10.

### Example 29

Preparation of Compound 267

3-Naphthalen-2-ylmethyl-6-phenyl-2-piperidin-4-yl-3H-pyrimidin-4-one (Compound 267):

The same procedure for the preaparation of compound 266 was repeated, starting from crude 4-(4-Hydroxy-6-phenyl-pyrimidin-2-yl)-piperidine-1-carboxylic acid tert-butyl ester and 2-bromomethylnaphthalene. Method A treatment of the intermediate then generated the TFA salt of 3-naphthalen-2-ylmethyl-6-phenyl-2-piperidin-4-yl-3H-pyrimidin-4-one 56. ¹H—NMR (500 MHz, methanol-d₄): δ 8.05 (s, 1H), 8.03-8.00 (m, 2H), 7.94-7.87 (m, 3H), 7.70-7.62 (m, 4H), 7.55 (s, 1H), 7.53-7.51 (m, 2H), 5.89 (s, 2H), 3.61-3.52 (m, 3H), 3.27-3.16 (m, 2H), 2.35-2.20 (m, 4H). HPLC ret. time: 6.59 min. LC/MS: (ES⁺, Cacld, for C₂₆H₂₅N₃O, Exact Mass: 395.20), Found, M+1 396.20.

### Example 30

Preparation of Compound 268

2,4-Bis-benzyloxy-5-(1,2,3,6-tetrahydro-pyridin-4-yl)-pyrimidine (Compound 268):

The TFA salt of 2,4-bis-benzyloxy-5-(1,2,3,6-tetrahydro-pyridin-4-yl)-pyrimidine was prepared from 2,4-bis-benzyloxy-5-bromo-pyrimidine and 4-(4,4,5,5-tetramethyl-[1,3,2]dioxa-borolan-2-yl)-3,6-dihydro-2H-pyridine-1-carboxylic acid tert-butyl ester, following the same procedure as for compound 250. ¹H—NMR (500 MHz, methanol-d₄): δ 8.20 (s, 1H), 7.49-7.35 (m, 10H), 6.01 (br s, 1H) 5.54 (s, 2H), 5.44 (s, 2H), 3.85 (br s, 2H), 3.34 (br s, 2H), 2.77 (br s, 2H). HPLC ret. time: 5.77 min. LC/MS:(ES⁺, Cacld,for C₂₃H₂₃N₃O₂, Exact Mass: 373.18), Found, M+1 374.10.

### Example 31

### Kᵢ Determination for the Inhibition of BACE

The ability of the inhibitors of the present invention to inhibit aspartic proteinases is demonstrated below using an assay that measures the inhibition of BACE. Compounds were tested against BACE activity using the following modifications of the method described in J. Ermolieff et al. (2000) Biochemistry 39(51):16263.

All compound evaluations were performed in 0.1M sodium acetate (buffer), pH, 4.5, 10 μM substrate (FS-1 peptide as described in the reference above; this is commercially available), varying concentrations of the test compound or control (DMSO to yeild 2% vol/vol), and 50 nM BACE. The assay volume is 100 μL.

Two microliters of the test compound dissolved in DMSO are added to each well in a 96-well microtiter plate. Seventy eight microliters of BACE are mixed with buffer and added to each well then incubated at room temperature for 15 minutes. A stock solution of 50 μM FS-1 substrate was prepared by addition of an aliquot of FS-1 substrate which was dissolved in DMSO to the buffer and mixed well. The reaction is initiated by addition of 20 μL of the FS-1 mix to the remaining, preincubated assay components. The cleavage reaction of substrate to product is measured using a Molecular Devices fluorescence plate reader with the excitation and emission filter pairs of 355 nm and 495 nm, respectively. Apparent Ki values are determined by fitting the data to the integrated equation for competitive tight binding inhibition.

The results are shown below in Table 5, wherein the following designations are used for the Kᵢ values:

TABLE 5

| | | |
|---|---|---|
| 100 | ** | Naphthalen-2-ylmethyl-(2-piperazin-1-yl- |
| | | 5-trifluoromethyl-phenyl)-amine |
| 101 | * | 4-Fluoro-naphthalene-1-carboxylic acid |
| | | (2-piperazin-1-yl-5-trifluoromethyl- |
| | | phenyl)-amide |
| 102 | ** | Isoquinoline-1-carboxylic acid (2- |
| | | piperazin-1-yl-5-trifluoromethyl-phenyl)- |
| | | amide |
| 103 | ** | Naphthalene-1-carboxylic acid (4′-fluoro- |
| | | 4-piperazin-1-yl-biphenyl-3-yl)-amide |
| 104 | *** | Naphthalene-1-carboxylic acid (3′-chloro- |
| | | 4′-fluoro-4-piperazin-1-yl-biphenyl-3- |
| | | yl)-amide |
| 105 | ** | Naphthalene-1-carboxylic acid (4′-fluoro- |
| | | 3′-formyl-4-piperazin-1-yl-biphenyl-3- |
| | | yl)-amide |
| 106 | *** | Naphthalene-1-carboxylic acid (2′,3′- |
| | | dichloro-4-piperazin-1-yl-biphenyl-3-yl)- |
| | | amide |
| 107 | *** | Naphthalene-1-carboxylic acid (2′,4′- |
| | | dichloro-4-piperazin-1-yl-biphenyl-3-yl)- |
| | | amide |
| 108 | *** | Naphthalene-1-carboxylic acid (2′,5′- |
| | | dichloro-4-piperazin-1-yl-biphenyl-3-yl)- |
| | | amide |
| 109 | *** | Naphthalene-1-carboxylic acid (2′,3′,5′- |
| | | trichloro-4-piperazin-1-yl-biphenyl-3- |
| | | yl)-amide |
| 110 | ** | Naphthalene-1-carboxylic acid (2- |
| | | piperazin-1-yl-5-pyridin-3-yl-phenyl)- |
| | | amide |
| 111 | ** | Naphthalene-1-carboxylic acid (2- |
| | | piperazin-1-yl-5-pyridin-4-yl-phenyl)- |
| | | amide |
| 112 | * | Naphthalene-1-carboxylic acid (5-bromo-4- |
| | | methyl-2-piperazin-1-yl-phenyl)-amide |
| 113 | ** | Naphthalene-2-carboxylic acid (2- |
| | | piperazin-1-yl-5-trifluoromethyl-phenyl)- |
| | | amide |
| 114 | ** | Naphthalene-1-carboxylic acid (2- |
| | | piperazin-1-yl-5-trifluoromethyl-phenyl)- |
| | | amide |
| 115 | ** | 4-{2,6-Bis-[(naphthalene-2-carbonyl)- |
| | | amino]-4-trifluoromethyl-phenyl}- |
| | | piperazine |
| 116 | *** | 1-[2,5-Bis-(2-trifluoromethyl- |
| | | phenoxymethyl)-phenyl]-piperazine |
| 117 | * | 4-tert-Butyl-N-(2-piperazin-1-yl-5- |
| | | trifluoromethyl-phenyl)-benzamide |
| 118 | * | Naphthalene-1-carboxylic acid (5-bromo-2- |
| | | piperazin-1-yl-phenyl)-amide |
| 119 | * | Naphthalene-1-carboxylic acid (3′- |
| | | methoxy-4-piperazin-1-yl-biphenyl-3-yl)- |
| | | amide |
| 120 | ** | Naphthalene-1-carboxylic acid (4′- |
| | | methoxy-4-piperazin-1-yl-biphenyl-3-yl)- |
| | | amide |
| 121 | ** | Naphthalene-1-carboxylic acid (4′-chloro- |
| | | 4-piperazin-1-yl-biphenyl-3-yl)-amide |
| 122 | ** | Naphthalene-1-carboxylic acid (2′-chloro- |
| | | 4-piperazin-1-yl-biphenyl-3-yl)-amide |
| 123 | ** | Naphthalene-1-carboxylic acid (3′-chloro- |
| | | 4-piperazin-1-yl-biphenyl-3-yl)-amide |
| 124 | ** | Naphthalene-1-carboxylic acid (4′-methyl- |
| | | 4-piperazin-1-yl-biphenyl-3-yl)-amide |
| 125 | ** | Naphthalene-1-carboxylic acid [2- |
| | | piperazin-1-yl-5-(2-trifluoromethyl- |
| | | phenoxymethyl)-phenyl]-amide |
| 126 | ** | Naphthalene-1-carboxylic acid (3′-methyl- |
| | | 4-piperazin-1-yl-biphenyl-3-yl)-amide |
| 127 | ** | 4-{2,6-Bis-[(naphthalene-1-carbonyl)- |
| | | amino]-4-trifluoromethyl-phenyl}- |
| | | piperazine |
| 128 | ** | Naphthalene-1-carboxylic acid (4- |
| | | piperazin-1-yl-3′-trifluoromethyl- |
| | | biphenyl-3-yl)-amide |
| 129 | *** | Naphthalene-1-carboxylic acid (4- |
| | | piperazin-1-yl-4′-trifluoromethyl- |
| | | biphenyl-3-yl)-amide |
| 130 | *** | Naphthalene-1-carboxylic acid (3′,4′- |
| | | dichloro-4-piperazin-1-yl-biphenyl-3-yl)- |
| | | amide |
| 131 | ** | Naphthalene-1-carboxylic acid (4′-cyano- |
| | | 4-piperazin-1-yl-biphenyl-3-yl)-amide |
| 132 | ** | Naphthalene-1-carboxylic acid (5-phenoxy- |
| | | 2-piperazin-1-yl-phenyl)-amide |
| 133 | ** | Naphthalene-1-carboxylic acid [5-(4- |
| | | chloro-phenoxy)-2-piperazin-1-yl-phenyl]- |
| | | amide |
| 134 | * | 2-Naphthalen-1-yl-N-(2-piperazin-1-yl-5- |
| | | trifluoromethyl-phenyl)-acetamide |
| 135 | * | Naphthalene-1-sulfonic acid (2-piperazin- |
| | | 1-yl-5-trifluoromethyl-phenyl)-amide |
| 136 | * | Naphthalene-2-sulfonic acid (2-piperazin- |
| | | 1-yl-5-trifluoromethyl-phenyl)-amide |
| 137 | ** | Biphenyl-4-sulfonic acid (2-piperazin-1- |
| | | yl-5-trifluoromethyl-phenyl)-amide |
| 138 | *** | Naphthalene-1-carboxylic acid (3′,4′- |
| | | dichloro-6-methyl-4-piperazin-1-yl- |
| | | biphenyl-3-yl)-amide |
| 139 | ** | Naphthalene-1-carboxylic acid [5-(3- |
| | | chloro-phenoxy)-2-piperazin-1-yl-phenyl]- |
| | | amide |
| 140 | ** | Naphthalene-1-carboxylic acid (2- |
| | | piperazin-1-yl-5-o-tolyloxy-phenyl)-amide |
| 141 | ** | Naphthalene-1-carboxylic acid (2- |
| | | piperazin-1-yl-5-m-tolyloxy-phenyl)-amide |
| 142 | ** | Naphthalene-1-carboxylic acid (2- |
| | | piperazin-1-yl-5-p-tolyloxy-phenyl)-amide |
| 143 | * | 6-Methoxy-naphthalene-1-carboxylic acid |
| | | (2-piperazin-1-yl-5-trifluoromethyl- |
| | | phenyl)-amide |
| 144 | ** | Naphthalene-1-carboxylic acid (4′- |
| | | isopropylsulfamoyl-4-piperazin-1-yl- |
| | | biphenyl-3-yl)-amide |
| 145 | ** | Naphthalene-1-carboxylic acid (4′- |
| | | diethylsulfamoyl-4-piperazin-1-yl- |
| | | biphenyl-3-yl)-amide |
| 146 | *** | Naphthalene-1-carboxylic acid (4′- |
| | | benzylsulfamoyl-4-piperazin-1-yl- |
| | | biphenyl-3-yl)-amide |
| 147 | *** | Naphthalene-1-carboxylic acid (4′- |
| | | cyclohexylsulfamoyl-4-piperazin-1-yl- |
| | | biphenyl-3-yl)-amide |
| 148 | * | Naphthalene-1-carboxylic acid (3-chloro- |
| | | 2-piperazin-1-yl-5-trifluoromethyl- |
| | | phenyl)-amide |
| 149 | ** | Quinoline-8-carboxylic acid (2-piperazin- |
| | | 1-yl-5-trifluoromethyl-phenyl)-amide |
| 150 | ** | (2-Piperazin-1-yl-5-trifluoromethyl- |
| | | phenyl)-carbamic acid naphthalen-1-yl |
| | | ester |
| 151 | ** | (2-Piperazin-1-yl-5-trifluoromethyl- |
| | | phenyl)-carbamic acid naphthalen-2-yl |
| | | ester |
| 152 | * | Naphthalene-1-carboxylic acid (5-furan-3- |
| | | yl-2-piperazin-1-yl-phenyl)-amide |
| 153 | ** | Naphthalene-1-carboxylic acid (2- |
| | | piperazin-1-yl-5-thiophen-3-yl-phenyl)- |
| | | amide |
| 154 | * | Naphthalene-1-carboxylic acid (5-furan-3- |
| | | yl-4-methyl-2-piperazin-1-yl-phenyl)- |
| | | amide |
| 155 | ** | Naphthalene-1-carboxylic acid (4-methyl- |
| | | 2-piperazin-1-yl-5-thiophen-3-yl-phenyl)- |
| | | amide |
| 156 | * | Naphthalene-1-carboxylic acid (4- |
| | | benzyloxy-2-piperazin-1-yl-phenyl)-amide |
| 157 | * | Naphthalene-1-carboxylic acid (4-bromo-5- |
| | | fluoro-2-piperazin-1-yl-phenyl)-amide |
| 158 | ** | Naphthalene-1-carboxylic acid (2-fluoro- |
| | | 5-piperazin-1-yl-biphenyl-4-yl)-amide |
| 159 | *** | Naphthalene-1-carboxylic acid (2-fluoro- |
| | | 5-piperazin-1-yl-4′-trifluoromethyl- |
| | | biphenyl-4-yl)-amide |
| 160 | ** | Naphthalene-1-carboxylic acid (5-fluoro- |
| | | 4-furan-3-yl-2-piperazin-1-yl-phenyl)- |
| | | amide |
| 161 | ** | Naphthalene-1-carboxylic acid (2′-fluoro- |
| | | 4-piperazin-1-yl-4′-trifluoromethyl- |
| | | biphenyl-3-yl)-amide |
| 162 | *** | Naphthalene-1-carboxylic acid (2′,5′- |
| | | difluoro-4-piperazin-1-yl-4′- |
| | | trifluoromethyl-biphenyl-3-yl)-amide |
| 163 | *** | Naphthalene-1-carboxylic acid (4′- |
| | | benzylsulfamoyl-3′-fluoro-4-piperazin-1- |
| | | yl-biphenyl-3-yl)-amide |
| 164 | ** | Naphthalene-1-carboxylic acid (4′- |
| | | benzylsulfamoyl-2′,5′-difluoro-4- |
| | | piperazin-1-yl-biphenyl-3-yl)-amide |
| 165 | *** | Naphthalen-2-ylmethyl-(4-piperazin-1-yl- |
| | | biphenyl-3-yl)-amine |
| 166 | *** | Naphthalen-2-ylmethyl-(4-piperazin-1-yl- |
| | | 4′-trifluoromethyl-biphenyl-3-yl)-amine |
| 167 | * | Naphthalene-1-carboxylic acid (4-chloro- |
| | | 2-piperazin-1-yl-5-trifluoromethyl- |
| | | phenyl)-amide |
| 168 | *** | Naphthalene-1-carboxylic acid (3′,4′- |
| | | dichloro-5-piperazin-1-yl-2- |
| | | trifluoromethyl-biphenyl-4-yl)-amide |
| 169 | *** | Naphthalene-1-carboxylic acid (2′,5′- |
| | | dichloro-5-piperazin-1-yl-2- |
| | | trifluoromethyl-biphenyl-4-yl)-amide |
| 170 | *** | Naphthalene-1-carboxylic acid (5- |
| | | piperazin-1-yl-2,4′-bis-trifluoromethyl- |
| | | biphenyl-4-yl)-amide |
| 171 | *** | 4′-Trifluoromethyl-biphenyl-4-sulfonic |
| | | acid (2-piperazin-1-yl-5-trifluoromethyl- |
| | | phenyl)-amide |
| 172 | *** | 2′-Trifluoromethyl-biphenyl-4-sulfonic |
| | | acid (2-piperazin-1-yl-5-trifluoromethyl- |
| | | phenyl)-amide |
| 173 | *** | Naphthalene-1-carboxylic acid (3′,4′- |
| | | dichloro-3-piperazin-1-yl-biphenyl-4-yl)- |
| | | amide |
| 174 | *** | Naphthalene-1-carboxylic acid (3- |
| | | piperazin-1-yl-4′-trifluoromethyl- |
| | | biphenyl-4-yl)-amide |
| 175 | *** | Naphthalene-1-carboxylic acid (3′,4′- |
| | | dichloro-2-fluoro-5-piperazin-1-yl- |
| | | biphenyl-4-yl)-amide |
| 176 | *** | Isoquinoline-1-carboxylic acid [5-bromo- |
| | | 2-piperazin-1-yl-3-(2-trifluoromethyl- |
| | | phenoxymethyl)-phenyl]-amide |
| 177 | *** | Isoquinoline-1-carboxylic acid [4- |
| | | piperazin-1-yl-5-(2-trifluoromethyl- |
| | | phenoxymethyl)-biphenyl-3-yl]-amide |
| 178 | *** | Isoquinoline-1-carboxylic acid [2- |
| | | piperazin-1-yl-4-(2-trifluoromethyl- |
| | | phenoxymethyl)-phenyl]-amide |
| 179 | ** | 4′-Trifluoromethyl-biphenyl-4-sulfonic |
| | | acid (3′,4′-dichloro-4-piperazin-1-yl- |
| | | biphenyl-3-yl)-amide |
| 180 | ** | 3′-Chloro-biphenyl-4-sulfonic acid |
| | | (3′,4′-dichloro-4-piperazin-1-yl- |
| | | biphenyl-3-yl)-amide |
| 181 | ** | 4′-Chloro-biphenyl-4-sulfonic acid |
| | | (3′,4′-dichloro-4-piperazin-1-yl- |
| | | biphenyl-3-yl)-amide |
| 182 | *** | 3′-Methyl-biphenyl-4-sulfonic acid |
| | | (3′,4′-dichloro-4-piperazin-1-yl- |
| | | biphenyl-3-yl)-amide |
| 182 | *** | 4′-Methyl-biphenyl-4-sulfonic acid |
| | | (3′,4′-dichloro-4-piperazin-1-yl- |
| | | biphenyl-3-yl)-amide |
| 183 | *** | Isoquinoline-1-carboxylic acid [5-bromo- |
| | | 2-piperazin-1-yl-4-(2-trifluoromethyl- |
| | | phenoxymethyl)-phenyl]-amide |
| 184 | *** | Isoquinoline-1-carboxylic acid [4- |
| | | piperazin-1-yl-6-(2-trifluoromethyl- |
| | | phenoxymethyl)-biphenyl-3-yl]-amide |
| 185 | ** | Isoquinoline-1-carboxylic acid [4- |
| | | piperazin-1-yl-4′-trifluoromethyl-6-(2- |
| | | trifluoromethyl-phenoxymethyl)-biphenyl- |
| | | 3-yl]-amide |
| 186 | *** | Isoquinoline-1-carboxylic acid [4′- |
| | | hydroxy-4-piperazin-1-yl-6-(2- |
| | | trifluoromethyl-phenoxymethyl)-biphenyl- |
| | | 3-yl]-amide |
| 187 | *** | Isoquinoline-1-carboxylic acid [5-furan- |
| | | 3-yl-2-piperazin-1-yl-4-(2- |
| | | trifluoromethyl-phenoxymethyl)-phenyl]- |
| | | amide |
| 188 | ** | 5-Bromo-2-piperazin-1-yl-3-[(quinolin-2- |
| | | ylmethyl)-amino]-benzoic acid ethyl ester |
| 189 | ** | Quinoxaline-2-carboxylic acid (2- |
| | | piperazin-1-yl-5-trifluoromethyl-phenyl)- |
| | | amide |
| 190 | ** | [1,6]Naphthyridine-2-carboxylic acid (2- |
| | | piperazin-1-yl-5-trifluoromethyl-phenyl)- |
| | | amide |
| 191 | ** | 4-{3-[(Naphthalen-2-ylmethyl)-amino]-4′- |
| | | trifluoromethyl-biphenyl-4-yl}- |
| | | piperazine-2-carboxylic acid |
| 192 | *** | 4-{3-[(Naphthalen-2-ylmethyl)-amino]-4′- |
| | | trifluoromethyl-biphenyl-4-yl}- |
| | | piperazine-2-carboxylic acid methyl ester |
| 193 | *** | 4-{3-[(Naphthalen-2-ylmethyl)-amino]-4′- |
| | | trifluoromethyl-biphenyl-4-yl}- |
| | | piperazine-2-carboxylic acid |
| | | isopropylamide |
| 194 | *** | 4-{3-[(Naphthalen-2-ylmethyl)-amino]-4′- |
| | | trifluoromethyl-biphenyl-4-yl}- |
| | | piperazine-2-carboxylic acid benzylamide |
| 195 | *** | 4-{3-[(Naphthalen-2-ylmethyl)-amino]-4′- |
| | | trifluoromethyl-biphenyl-4-yl}- |
| | | piperazine-2-carboxylic acid |
| | | dimethylamide |
| 200 | * | 1-[4-(4-Chloro-2-methyl-phenoxy)- |
| | | butyryl]-piperazine-2-carboxylic acid |
| | | naphthalen-2-ylamide |
| 201 | * | Naphthalene-1-carboxylic acid (2- |
| | | [1,4]diazepan-1-yl-5-trifluoromethyl- |
| | | phenyl)-amide |
| 202 | * | 1,2,3,4,5,6-Hexahydro-azepino[4,5- |
| | | b]indole-5-carboxylic acid naphthalen-2- |
| | | ylamide |
| 203 | * | 4-[4-(2-Trifluoromethyl-phenoxymethyl)- |
| | | phenyl]-piperidine-3-carboxylic acid |
| | | (furan-2-ylmethyl)-amide |
| 204 | * | 4-[4-(2-Trifluoromethyl-phenoxymethyl)- |
| | | phenyl]-piperidine-3-carboxylic acid |
| | | phenylamide |
| 205 | * | (3,4-Dihydro-1H-isoquinolin-2-yl)-{4-[4- |
| | | (2-trifluoromethyl-phenoxymethyl)- |
| | | phenyl]-piperidin-3-yl}-methanone |
| 206 | * | 1-[3-(2-Trifluoromethyl-phenoxymethyl)- |
| | | benzoyl]-piperazine-2-carboxylic acid |
| | | naphthalen-2-ylamide |
| 207 | * | 2-({4-[4-(2-Trifluoromethyl- |
| | | phenoxymethyl)-phenyl]-piperidin-3- |
| | | ylmethyl}-carbamoyl)- |
| | | cyclohexanecarboxylic acid |
| 208 | * | 4-[4-(2-Trifluoromethyl-phenoxymethyl)- |
| | | phenyl]-piperidine-3-carboxylic acid 2- |
| | | trifluoromethoxy-benzylamide |
| 209 | ** | 4-[4-(2-Trifluoromethyl-phenoxymethyl)- |
| | | phenyl]-piperidine-3-carboxylic acid |
| | | (1,2,3,4-tetrahydro-naphthalen-1-yl)- |
| | | amide |
| 210 | ** | 2,4-Bis-benzyloxy-5-(1,2,3,6-tetrahydro- |
| | | pyridin-4-yl)-pyrimidine |
| 211 | ** | 4-[4-(2-Trifluoromethyl-phenoxymethyl)- |
| | | phenyl]-piperidine-3-carboxylic acid |
| | | benzhydryl-amide |
| 212 | ** | 2-{4-[4-(2-Trifluoromethyl- |
| | | phenoxymethyl)-phenyl]-piperidin-3- |
| | | ylmethyl}-isoindole-1,3-dione |
| 213 | ** | 3-({4-[4-(2-Trifluoromethyl- |
| | | phenoxymethyl)-phenyl]-piperidin-3- |
| | | ylmethyl}-carbamoyl)-naphthalene-2- |
| | | carboxylic acid |
| 214 | ** | 6-Phenyl-2-piperidin-4-yl-3-(2- |
| | | trifluoromethyl-benzyl)-3H-pyrimidin-4- |
| | | one |
| 215 | ** | 4-[4-(2-Trifluoromethyl-phenoxymethyl)- |
| | | phenyl]-piperidine-3-carboxylic acid |
| | | (naphthalen-1-ylmethyl)-amide |
| 216 | ** | 4-[4-(2-Trifluoromethyl-phenoxymethyl)- |
| | | phenyl]-piperidine-3-carboxylic acid |
| | | naphthalen-2-ylamide |
| 217 | ** | Naphthalene-1-carboxylic acid [2-(3,4- |
| | | dichloro-phenyl)-4-piperazin-1-yl- |
| | | pyrimidin-5-yl]-amide |
| 218 | ** | 3-Naphthalen-2-ylmethyl-6-phenyl-2- |
| | | piperidin-4-yl-3H-pyrimidin-4-one |
| 219 | ** | 4-[4-(2-Trifluoromethyl-phenoxymethyl)- |
| | | phenyl]-piperidine-3-carboxylic acid |
| | | (1,2,3,4-tetrahydro-naphthalen-2-yl)- |
| | | amide |
| 220 | ** | 4-[4-(2-Trifluoromethyl-phenoxymethyl)- |
| | | phenyl]-piperidine-3-carboxylic acid |
| | | benzyl-naphthalen-2-yl-amide |
| 221 | ** | Naphthalene-1-carboxylic acid {4-[4-(2- |
| | | trifluoromethyl-phenoxymethyl)-phenyl]- |
| | | piperidin-3-ylmethyl}-amide |
| 222 | ** | Naphthalene-2-carboxylic acid {4-[4-(2- |
| | | trifluoromethyl-phenoxymethyl)-phenyl]- |
| | | piperidin-3-ylmethyl}-amide |
| 223 | ** | {1-Benzyl-2-oxo-2-[2-({4-[4-(2- |
| | | trifluoromethyl-phenoxymethyl)-phenyl]- |
| | | piperidin-3-ylmethyl}-carbamoyl)- |
| | | pyrrolidin-1-yl]-ethyl}-carbamic acid |
| | | benzyl ester |
| 224 | ** | 1-Naphthalen-1-yl-3-{4-[4-(2- |
| | | trifluoromethyl-phenoxymethyl)-phenyl]- |
| | | piperidin-3-ylmethyl}-urea |
| 225 | ** | (2-Phenyl-1-{[({4-[4-(2-trifluoromethyl- |
| | | phenoxymethyl)-phenyl]-piperidin-3- |
| | | ylmethyl}-carbamoyl)-methyl]-carbamoyl}- |
| | | ethyl)-carbamic acid benzyl ester |
| 226 | *** | N4-Methyl-N4-(2-methylamino-ethyl)-N3- |
| | | naphthalen-2-ylmethyl-4′-trifluoromethyl- |
| | | biphenyl-3,4-diamine |
| 227 | *** | Naphthalene-1-carboxylic acid [6-(3,4- |
| | | dichloro-phenyl)-2-piperazin-1-yl- |
| | | pyridin-3-yl]-amide |
| 228 | *** | {4-[4-(2-Trifluoromethyl-phenoxymethyl)- |
| | | phenyl]-piperidin-3-ylmethyl}-carbamic |
| | | acid naphthalen-2-yl ester |
| 229 | *** | {4-[4-(2-Trifluoromethyl-phenoxymethyl)- |
| | | phenyl]-piperidin-3-ylmethyl}-carbamic |
| | | acid naphthalen-1-yl ester |
| 230 | *** | {1-(1H-Indol-3-ylmethyl)-2-oxo-2-[2-({4- |
| | | [4-(2-trifluoromethyl-phenoxymethyl)- |
| | | phenyl]-piperidin-3-ylmethyl}-carbamoyl)- |
| | | pyrrolidin-1-yl]-ethyl}-carbamic acid 9H- |
| | | fluoren-9-ylmethyl ester |
| 231 | *** | Naphthalene-2-sulfonic acid {4-[4-(2- |
| | | trifluoromethyl-phenoxymethyl)-phenyl]- |
| | | piperidin-3-ylmethyl}-amide |
| 232 | *** | 1-Naphthalen-2-yl-3-{4-[4-(2- |
| | | trifluoromethyl-phenoxymethyl)-phenyl]- |
| | | piperidin-3-ylmethyl}-urea |
| 301 | *** | 4-[4-Naphthalen-1-yl-2,5-bis-(2- |
| | | trifluoromethyl-phenoxymethyl)-phenyl]- |
| | | 1,2,3,6-tetrahydro-pyridine |
| 302 | *** | 4-Biphenyl-4-yl-3-(naphthalen-2- |
| | | yloxymethyl)-1,2,3,6-tetrahydro-pyridine |
| 303 | *** | 4-[2,5-Bis-(2-trifluoromethyl- |
| | | phenoxymethyl)-phenyl]-1,2,3,6- |
| | | tetrahydro-pyridine |
| 304 | *** | 4-[2,6-Bis-(2-trifluoromethyl- |
| | | phenoxymethyl)-phenyl]-1,2,3,6- |
| | | tetrahydro-pyridine |
| 305 | ** | 6-Benzyloxy-9-naphthalen-2-ylmethyl- |
| | | 2,3,4,9-tetrahydro-1H-b-carboline |
| 306 | *** | 4-[2,5-Bis-(2-trifluoromethyl- |
| | | phenoxymethyl)-biphenyl-4-yl]-1,2,3,6- |
| | | tetrahydro-pyridine |
| 307 | *** | 4-[2,5-Bis-(naphthalen-2-yloxymethyl)- |
| | | biphenyl-4-yl]-1,2,3,6-tetrahydro- |
| | | pyridine |
| 308 | ** | N-Naphthalen-2-yl-2-(1,2,3,6-tetrahydro- |
| | | pyridin-4-yl)-4-(2-trifluoromethyl- |
| | | phenoxymethyl)-benzamide |
| 309 | ** | N-(4-Methoxy-naphthalen-2-yl)-2-(1,2,3,6- |
| | | tetrahydro-pyridin-4-yl)-4-(2- |
| | | trifluoromethyl-phenoxymethyl)-benzamide |
| 310 | ** | N-(5-Amino-naphthalen-1-yl)-2-(1,2,3,6- |
| | | tetrahydro-pyridin-4-yl)-4-(2- |
| | | trifluoromethyl-phenoxymethyl)-benzamide |
| 311 | ** | N-(3-Amino-naphthalen-2-yl)-2-(1,2,3,6- |
| | | tetrahydro-pyridin-4-yl)-4-(2- |
| | | trifluoromethyl-phenoxymethyl)-benzamide |
| 312 | *** | Naphthalene-1-carboxylic acid 2-(1,2,3,6- |
| | | tetrahydro-pyridin-4-yl)-4-(2- |
| | | trifluoromethyl-phenoxymethyl)-benzyl |
| | | ester |
| 313 | *** | Naphthalene-2-carboxylic acid 2-(1,2,3,6- |
| | | tetrahydro-pyridin-4-yl)-4-(2- |
| | | trifluoromethyl-phenoxymethyl)-benzyl |
| | | ester |
| 314 | ** | 2-Trifluoromethyl-benzoic acid 2- |
| | | (1,2,3,6-tetrahydro-pyridin-4-yl)-4-(2- |
| | | trifluoromethyl-phenoxymethyl)-benzyl |
| | | ester |
| 315 | ** | Benzyloxy-acetic acid 2-(1,2,3,6- |
| | | tetrahydro-pyridin-4-yl)-4-(2- |
| | | trifluoromethyl-phenoxymethyl)-benzyl |
| | | ester |
| 316 | ** | Benzo[1,3]dioxole-5-carboxylic acid 2- |
| | | (1,2,3,6-tetrahydro-pyridin-4-yl)-4-(2- |
| | | trifluoromethyl-phenoxymethyl)-benzyl |
| | | ester |
| 317 | ** | Terephthalic acid 1-methyl ester 4-[2- |
| | | (1,2,3,6-tetrahydro-pyridin-4-yl)-4-(2- |
| | | trifluoromethyl-phenoxymethyl)-benzyl] |
| | | ester |
| 318 | *** | Carbonic acid naphthalen-1-yl ester 2- |
| | | (1,2,3,6-tetrahydro-pyridin-4-yl)-4-(2- |
| | | trifluoromethyl-phenoxymethyl)-benzyl |
| | | ester |
| 319 | *** | Carbonic acid naphthalen-2-yl ester 2- |
| | | (1,2,3,6-tetrahydro-pyridin-4-yl)-4-(2- |
| | | trifluoromethyl-phenoxymethyl)-benzyl |
| | | ester |
| 320 | ** | 4-[2-(Naphthalen-1-yloxymethyl)-5-(2- |
| | | trifluoromethyl-phenoxymethyl)-phenyl]- |
| | | 1,2,3,6-tetrahydro-pyridine |
| 321 | *** | 4-[2-(Naphthalen-2-yloxymethyl)-5-(2- |
| | | trifluoromethyl-phenoxymethyl)-phenyl]- |
| | | 1,2,3,6-tetrahydro-pyridine |
| 322 | ** | N-Naphthalen-1-yl-2-(1,2,3,6-tetrahydro- |
| | | pyridin-4-yl)-4-(2-trifluoromethyl- |
| | | phenoxymethyl)-benzamide |
| 323 | *** | 4-[5-(2-Trifluoromethyl-phenoxymethyl)-2- |
| | | (4-trifluoromethyl-phenoxymethyl)- |
| | | phenyl]-1,2,3,6-tetrahydro-pyridine |
| 324 | *** | 4-[5-(2-Trifluoromethyl-phenoxymethyl)-2- |
| | | (3-trifluoromethyl-phenoxymethyl)- |
| | | phenyl]-1,2,3,6-tetrahydro-pyridine |
| 325 | *** | 4-[2-(Biphenyl-4-yloxymethyl)-5-(2- |
| | | trifluoromethyl-phenoxymethyl)-phenyl]- |
| | | 1,2,3,6-tetrahydro-pyridine |
| 326 | ** | 4-[2-([1,1′;3′,1′′]Terphenyl-4′- |
| | | yloxymethyl)-5-(2-trifluoromethyl- |
| | | phenoxymethyl)-phenyl]-1,2,3,6- |
| | | tetrahydro-pyridine |
| 327 | *** | 5-[2-(1,2,3,6-Tetrahydro-pyridin-4-yl)-4- |
| | | (2-trifluoromethyl-phenoxymethyl)- |
| | | benzyloxy]-quinoline |
| 328 | ** | 3-[2-(1,2,3,6-Tetrahydro-pyridin-4-yl)-4- |
| | | (2-trifluoromethyl-phenoxymethyl)- |
| | | benzyloxy]-benzoic acid methyl ester |
| 329 | ** | 4-[2-(1,2,3,6-Tetrahydro-pyridin-4-yl)-4- |
| | | (2-trifluoromethyl-phenoxymethyl)- |
| | | benzyloxy]-benzole acid methyl ester |
| 330 | *** | 5-[2-(1,2,3,6-Tetrahydro-pyridin-4-yl)-4- |
| | | (2-trifluoromethyl-phenoxymethyl)- |
| | | benzyloxy]-isophthalic acid dimethyl |
| | | ester |
| 331 | *** | 5-[2-(1,2,3,6-Tetrahydro-pyridin-4-yl)-4- |
| | | (2-trifluoromethyl-phenoxymethyl)- |
| | | benzyloxy]-3,4-dihydro-2H-naphthalen-1- |
| | | one |
| 332 | *** | 2-Methyl-5-[2-(1,2,3,6-tetrahydro- |
| | | pyridin-4-yl)-4-(2-trifluoromethyl- |
| | | phenoxymethyl)-benzyloxy]-1H-indole-3- |
| | | carboxylic acid ethyl ester |
| 333 | *** | 4-[4-Bromo-2,5-bis-(2-trifluoromethyl- |
| | | phenoxymethyl)-phenyl]-1,2,3,6- |
| | | tetrahydro-pyridine |
| 334 | ** | 4-[4-(1,2,3,6-Tetrahydro-pyridin-4-yl)- |
| | | 2,5-bis-(2-trifluoromethyl- |
| | | phenoxymethyl)-phenyl]-1,2,3,6- |
| | | tetrahydro-pyridine |
| 335 | ** | 4-[3′,4′-Dichloro-2,5-bis-(2- |
| | | trifluoromethyl-phenoxymethyl)-biphenyl- |
| | | 4-yl]-1,2,3,6-tetrahydro-pyridine |
| 336 | *** | 4-[2′-Trifluoromethyl-2,5-bis-(2- |
| | | trifluoromethyl-phenoxymethyl)-biphenyl- |
| | | 4-yl]-1,2,3,6-tetrahydro-pyridine |
| 337 | ** | 4-[3′-Trifluoromethyl-2,5-bis-(2- |
| | | trifluoromethyl-phenoxymethyl)-biphenyl- |
| | | 4-yl]-1,2,3,6-tetrahydro-pyridine |
| 338 | ** | 4-[4′-Trifluoromethyl-2,5-bis-(2- |
| | | trifluoromethyl-phenoxymethyl)-biphenyl- |
| | | 4-yl]-1,2,3,6-tetrahydro-pyridine |
| 339 | ** | 4-[4-Naphthalen-2-yl-2,5-bis-(2- |
| | | trifluoromethyl-phenoxymethyl)-phenyl]- |
| | | 1,2,3,6-tetrahydro-pyridine |
| 340 | *** | 3-[4-(1,2,3,6-Tetrahydro-pyridin-4-yl)- |
| | | 2,5-bis-(2-trifluoromethyl- |
| | | phenoxymethyl)-phenyl]-pyridine |
| 341 | *** | 4-[4-(1,2,3,6-Tetrahydro-pyridin-4-yl)- |
| | | 2,5-bis-(2-trifluoromethyl- |
| | | phenoxymethyl)-phenyl]-pyridine |
| 342 | *** | 4-[4-Thiophen-3-yl-2,5-bis-(2- |
| | | trifluoromethyl-phenoxymethyl)-phenyl]- |
| | | 1,2,3,6-tetrahydro-pyridine |
| 343 | *** | 4-[4-Furan-3-yl-2,5-bis-(2- |
| | | trifluoromethyl-phenoxymethyl)-phenyl]- |
| | | 1,2,3,6-tetrahydro-pyridine |
| 344 | *** | 4-[2′-Nitro-2,5-bis-(2-trifluoromethyl- |
| | | phenoxymethyl)-biphenyl-4-yl]-1,2,3,6- |
| | | tetrahydro-pyridine |
| 345 | *** | 4-[4-Thiophen-2-yl-2,5-bis-(2- |
| | | trifluoromethyl-phenoxymethyl)-phenyl]- |
| | | 1,2,3,6-tetrahydro-pyridine |
| 346 | *** | 4-[4-Furan-2-yl-2,5-bis-(2- |
| | | trifluoromethyl-phenoxymethyl)-phenyl]- |
| | | 1,2,3,6-tetrahydro-pyridine |
| 347 | *** | 4-[2′-Fluoro-2,5-bis-(2-trifluoromethyl- |
| | | phenoxymethyl)-biphenyl-4-yl]-1,2,3,6- |
| | | tetrahydro-pyridine |
| 348 | *** | 4-[2′-Chloro-2,5-bis-(2-trifluoromethyl- |
| | | phenoxymethyl)-biphenyl-4-yl]-1,2,3,6- |
| | | tetrahydro-pyridine |
| 349 | *** | 4-[2′,6′-Difluoro-2,5-bis-(2- |
| | | trifluoromethyl-phenoxymethyl)-biphenyl- |
| | | 4-yl]-1,2,3,6-tetrahydro-pyridine |
| 350 | *** | 1-[4′-(1,2,3,6-Tetrahydro-pyridin-4-yl)- |
| | | 2′,5′-bis-(2-trifluoromethyl- |
| | | phenoxymethyl)-biphenyl-2-yl]-ethanone |
| 351 | *** | 4′-(1,2,3,6-Tetrahydro-pyridin-4-yl)- |
| | | 2′,5′-bis-(2-trifluoromethyl- |
| | | phenoxymethyl)-biphenyl-3-ol |
| 352 | *** | 4′-(1,2,3,6-Tetrahydro-pyridin-4-yl)- |
| | | 2′,5′-bis-(2-trifluoromethyl- |
| | | phenoxymethyl)-biphenyl-4-ol |
| 353 | *** | 4-[3′-Nitro-2,5-bis-(2-trifluoromethyl- |
| | | phenoxymethyl)-biphenyl-4-yl]-1,2,3,6- |
| | | tetrahydro-pyridine |
| 354 | *** | 4-[4′-Nitro-2,5-bis-(2-trifluoromethyl- |
| | | phenoxymethyl)-biphenyl-4-yl]-1,2,3,6- |
| | | tetrahydro-pyridine |
| 355 | *** | 1-[4′-(1,2,3,6-Tetrahydro-pyridin-4-yl)- |
| | | 2′,5′-bis-(2-trifluoromethyl- |
| | | phenoxymethyl)-biphenyl-2-yl]-ethanol |
| 356 | *** | 4-[2,4,5-Tris-(2-trifluoromethyl- |
| | | phenoxymethyl)-phenyl]-1,2,3,6- |
| | | tetrahydro-pyridine |
| 357 | *** | 4-[4-Benzofuran-2-yl-2,5-bis-(2- |
| | | trifluoromethyl-phenoxymethyl)-phenyl]- |
| | | 1,2,3,6-tetrahydro-pyridine |
| 358 | *** | 4-[4-(1H-Pyrrol-2-yl)-2,5-bis-(2- |
| | | trifluoromethyl-phenoxymethyl)-phenyl]- |
| | | 1,2,3,6-tetrahydro-pyridine |
| 359 | *** | 4′-(1,2,3,6-Tetrahydro-pyridin-4-yl)- |
| | | 2′,5′-bis-(2-trifluoromethyl- |
| | | phenoxymethyl)-biphenyl-4-ylamine |
| 360 | *** | 4-[3-(2-Trifluoromethyl-phenoxymethyl)- |
| | | biphenyl-4-yl]-1,2,3,6-tetrahydro- |
| | | pyridine |
| 361 | ** | 4′-(1,2,3,6-Tetrahydro-pyridin-4-yl)-3′- |
| | | (2-trifluoromethyl-phenoxymethyl)- |
| | | biphenyl-4-ol |
| 362 | ** | 4′-(1,2,3,6-Tetrahydro-pyridin-4-yl)-3′- |
| | | (2-trifluoromethyl-phenoxymethyl)- |
| | | biphenyl-2-ol |
| 363 | ** | 4-[4-Furan-3-yl-2-(2-trifluoromethyl- |
| | | phenoxymethyl)-phenyl]-1,2,3,6- |
| | | tetrahydro-pyridine |
| 364 | *** | 4′-(1,2,3,6-Tetrahydro-pyridin-4-yl)- |
| | | 2′,5′-bis-(2-trifluoromethyl- |
| | | phenoxymethyl)-biphenyl-3-carboxylic acid |
| | | amide |
| 365 | *** | 4-[4′-Methoxy-2,5-bis-(2-trifluoromethyl- |
| | | phenoxymethyl)-biphenyl-4-yl]-1,2,3,6- |
| | | tetrahydro-pyridine |
| 366 | *** | [4′-(1,2,3,6-Tetrahydro-pyridin-4-yl)- |
| | | 2′,5′-bis-(2-trifluoromethyl- |
| | | phenoxymethyl)-biphenyl-4-yl]-methanol |
| 367 | *** | [4′-(1,2,3,6-Tetrahydro-pyridin-4-yl)- |
| | | 2′,5′-bis-(2-trifluoromethyl- |
| | | phenoxymethyl)-biphenyl-2-yl]-methanol |
| 368 | *** | 4′-(1,2,3,6-Tetrahydro-pyridin-4-yl)- |
| | | 2′,5′-bis-(2-trifluoromethyl- |
| | | phenoxymethyl)-biphenyl-3-carboxylic acid |
| | | methyl ester |
| 369 | *** | 4′-(1,2,3,6-Tetrahydro-pyridin-4-yl)- |
| | | 2′,5′-bis-(2-trifluoromethyl- |
| | | phenoxymethyl)-biphenyl-4-carboxylic acid |
| | | methyl ester |
| 370 | *** | Furan-2-carboxylic acid 4′-(1,2,3,6- |
| | | tetrahydro-pyridin-4-yl)-2′,5′-bis-(2- |
| | | trifluoromethyl-phenoxymethyl)-biphenyl- |
| | | 2-ylmethyl ester |
| 371 | ** | 4-[4-(1,2,3,6-Tetrahydro-pyridin-4-yl)-2- |
| | | (2-trifluoromethyl-phenoxymethyl)- |
| | | phenyl]-1,2,5,6-tetrahydro-pyridine |
| 372 | *** | 4-[2′-Fluoro-3-(2-trifluoromethyl- |
| | | phenoxymethyl)-biphenyl-4-yl]-1,2,3,6- |
| | | tetrahydro-pyridine |
| 373 | *** | 4-[2′-Chloro-3-(2-trifluoromethyl- |
| | | phenoxymethyl)-biphenyl-4-yl]-1,2,3,6- |
| | | tetrahydro-pyridine |
| 374 | *** | 4-[2′-Methyl-3-(2-trifluoromethyl- |
| | | phenoxymethyl)-biphenyl-4-yl]-1,2,3,6- |
| | | tetrahydro-pyridine |
| 375 | *** | 4-[2′-Trifluoromethyl-3-(2- |
| | | trifluoromethyl-phenoxymethyl)-biphenyl- |
| | | 4-yl]-1,2,3,6-tetrahydro-pyridine |
| 376 | ** | 4′-(1,2,3,6-Tetrahydro-pyridin-4-yl)-3′- |
| | | (2-trifluoromethyl-phenoxymethyl)- |
| | | biphenyl-2-ylamine |
| 377 | ** | 4-[4-Bromo-2-(2-trifluoromethyl- |
| | | phenoxymethyl)-phenyl]-1,2,3,6- |
| | | tetrahydro-pyridine |
| 378 | ** | [4′-(1,2,3,6-Tetrahydro-pyridin-4-yl)-3′- |
| | | (2-trifluoromethyl-phenoxymethyl)- |
| | | biphenyl-2-yl]-methanol |
| 379 | *** | Benzoic acid 4′-(1,2,3,6-tetrahydro- |
| | | pyridin-4-yl)-3′-(2-trifluoromethyl- |
| | | phenoxymethyl)-biphenyl-2-yl methyl ester |
| 380 | *** | 2-Trifluoromethyl-benzoic acid 4′- |
| | | (1,2,3,6-tetrahydro-pyridin-4-yl)-3′-(2- |
| | | trifluoromethyl-phenoxymethyl)-biphenyl- |
| | | 2-ylmethyl ester |
| 381 | ** | 2-Bromo-5-(1,2,3,6-tetrahydro-pyridin-4- |
| | | yl)-4-(2-trifluoromethyl-phenoxymethyl)- |
| | | benzole acid methyl ester |
| 382 | ** | 2,5-Bis-(1,2,3,6-tetrahydro-pyridin-4- |
| | | yl)-4-(2-trifluoromethyl-phenoxymethyl)- |
| | | benzoic acid methyl ester |
| 383 | ** | 2-Furan-3-yl-5-(1,2,3,6-tetrahydro- |
| | | pyridin-4-yl)-4-(2-trifluoromethyl- |
| | | phenoxymethyl)-benzoic acid methyl ester |
| 384 | *** | 2-Chloro-nicotinic acid 4′-(1,2,3,6- |
| | | tetrahydro-pyridin-4-yl)-3′-(2- |
| | | trifluoromethyl-phenoxymethyl)-biphenyl- |
| | | 2-ylmethyl ester |
| 385 | *** | Nicotinic acid 2-furan-3-yl-5-(1,2,3,6- |
| | | tetrahydro-pyridin-4-yl)-4-(2- |
| | | trifluoromethyl-phenoxymethyl)-benzyl |
| | | ester |
| 386 | *** | 2-Chloro-nicotinic acid 2-furan-3-yl-5- |
| | | (1,2,3,6-tetrahydro-pyridin-4-yl)-4-(2- |
| | | trifluoromethyl-phenoxymethyl)-benzyl |
| | | ester |
| 387 | ** | [2-Furan-3-yl-5-(1,2,3,6-tetrahydro- |
| | | pyridin-4-yl)-4-(2-trifluoromethyl- |
| | | phenoxymethyl)-phenyl]-methanol |
| 388 | ** | [2-Furan-3-yl-5-(1-methyl-1,2,3,6- |
| | | tetrahydro-pyridin-4-yl)-4-(2- |
| | | trifluoromethyl-phenoxymethyl)-phenyl]- |
| | | methanol |
| 389 | ** | Pyridine-2-carboxylic acid 2-furan-3-yl- |
| | | 5-(1,2,3,6-tetrahydro-pyridin-4-yl)-4-(2- |
| | | trifluoromethyl-phenoxymethyl)-benzyl |
| | | ester |
| 390 | *** | Isonicotinic acid 2-furan-3-yl-5- |
| | | (1,2,3,6-tetrahydro-pyridin-4-yl)-4-(2- |
| | | trifluoromethyl-phenoxymethyl)-benzyl |
| | | ester |
| 401 | *** | 4-Biphenyl-4-yl-3-(naphthalen-2- |
| | | ylmethoxy)-piperidine |
| 402 | *** | 4-Biphenyl-4-yl-piperidine-3-carboxylic |
| | | acid naphthalen-2-ylamide |
| 403 | ** | 1-(4-Biphenyl-4-yl-piperidin-3-yl)-3- |
| | | naphthalen-2-yl-urea |
| 404 | ** | 4-Biphenyl-4-yl-piperidine-3-carboxylic |
| | | acid (1-naphthalen-1-yl-ethyl)-amide |
| 405 | ** | 4-Biphenyl-4-yl-piperidine-3-carboxylic |
| | | acid (1-naphthalen-1-yl-ethyl)-amide |
| 406 | ** | 4-Biphenyl-4-yl-piperidine-3-carboxylic |
| | | acid (1-naphthalen-1-yl-ethyl)-amide |
| 407 | ** | 4-Biphenyl-4-yl-piperidine-3-carboxylic |
| | | acid (1-naphthalen-1-yl-ethyl)-amide |
| 408 | ** | 4-Biphenyl-4-yl-piperidine-3-carboxylic |
| | | acid (l-naphthalen-2-yl-ethyl)-amide |
| 409 | ** | 4-Biphenyl-4-yl-piperidine-3-carboxylic |
| | | acid (l-naphthalen-2-yl-ethyl)-amide |
| 410 | ** | 4-Biphenyl-4-yl-piperidine-3-carboxylic |
| | | acid (l-naphthalen-2-yl-ethyl)-amide |
| 411 | ** | 4-Biphenyl-4-yl-piperidine-3-carboxylic |
| | | acid (l-naphthalen-2-yl-ethyl)-amide |
| 412 | ** | 4-Biphenyl-4-yl-5-(naphthalen-2- |
| | | yloxymethyl)-piperidine-3,4-diol |
| 413 | *** | 4-Biphenyl-4-yl-3-(naphthalen-2- |
| | | yloxymethyl)-5-(3-trifluoromethyl- |
| | | benzyloxy)-piperidine |
| 501 | ** | 6-Benzyloxy-9-naphthalen-2-ylmethyl- |
| | | 2,3,4,9-tetrahydro-1H-b-carboline |
| 502 | * | (6-Methoxy-1,2,3,4-tetrahydro-b-carbolin- |
| | | 9-yl)-naphthalen-2-yl-methanone |
| 503 | * | 6-Methoxy-9-naphthalen-2-ylmethyl- |
| | | 2,3,4,9-tetrahydro-1H-b-carboline |
| 504 | *** | Naphthalen-1-yl-[6-(2-trifluoromethyl- |
| | | benzyloxy)-1,2,3,4-tetrahydro-b-carbolin- |
| | | 9-yl]-methanone |
| 505 | *** | 9-Naphthalen-1-ylmethyl-6-(2- |
| | | trifluoromethyl-benzyloxy)-2,3,4,9- |
| | | tetrahydro-1H-b-carboline |
| 506 | *** | Naphthalen-1-yl-[6-(4-trifluoromethyl- |
| | | benzyloxy)-1,2,3,4-tetrahydro-b-carbolin- |
| | | 9-yl]-methanone |
| 507 | *** | Naphthalen-2-yl-[6-(3-trifluoromethyl- |
| | | benzyloxy)-1,2,3,4-tetrahydro-b-carbolin- |
| | | 9-yl]-methanone |
| 508 | *** | Naphthalen-1-yl-[6-(3-trifluoromethyl- |
| | | benzyloxy)-1,2,3,4-tetrahydro-b-carbolin- |
| | | 9-yl]-methanone |
| 509 | *** | 9-Naphthalen-1-ylmethyl-6-(3- |
| | | trifluoromethyl-benzyloxy)-2,3,4,9- |
| | | tetrahydro-1H-b-carboline |
| 510 | *** | [6-(2-Chloro-5-trifluoromethyl- |
| | | benzyloxy)-1,2,3,4-tetrahydro-b-carbolin- |
| | | 9-yl]-naphthalen-1-yl-methanone |
| 511 | *** | [6-(2-Chloro-5-trifluoromethyl- |
| | | benzyloxy)-1,2,3,4-tetrahydro-b-carbolin- |
| | | 9-yl]-naphthalen-2-yl-methanone |
| 512 | *** | 6-(4-Difluoromethoxy-benzyloxy)-9- |
| | | naphthalen-1-ylmethyl-2,3,4,9-tetrahydro- |
| | | 1H-b-carboline |
| 513 | *** | 6-(4-Difluoromethoxy-benzyloxy)-9- |
| | | naphthalen-2-ylmethyl-2,3,4,9-tetrahydro- |
| | | 1H-b-carboline |
| 514 | *** | [6-(4-Difluoromethoxy-benzyloxy)-1,2,3,4- |
| | | tetrahydro-b-carbolin-9-yl]-naphthalen-1- |
| | | yl-methanone |
| 515 | *** | [6-(4-Difluoromethoxy-benzyloxy)-1,2,3,4- |
| | | tetrahydro-b-carbolin-9-yl]-naphthalen-2- |
| | | yl-methanone |
| 516 | *** | 6-(2-Difluoromethoxy-benzyloxy)-9- |
| | | naphthalen-2-ylmethyl-2,3,4,9-tetrahydro- |
| | | 1H-b-carboline |
| 517 | *** | [6-(2,5-Bis-trifluoromethyl-benzyloxy)- |
| | | 1,2,3,4-tetrahydro-b-carbolin-9-yl]- |
| | | naphthalen-1-yl-methanone |
| 518 | *** | 6-(2-Difluoromethoxy-benzyloxy)-9- |
| | | naphthalen-1-ylmethyl-2,3,4,9-tetrahydro- |
| | | 1H-b-carboline |
| 519 | *** | 6-(Naphthalen-2-ylmethoxy)-9-naphthalen- |
| | | 2-ylmethyl-2,3,4,9-tetrahydro-1H-b- |
| | | carboline |
| 520 | *** | 6-(2-Iodo-benzyloxy)-9-naphthalen-1- |
| | | ylmethyl-2,3,4,9-tetrahydro-1H-b- |
| | | carboline |
| 521 | *** | 6-(2-Methyl-3-trifluoromethyl-benzyloxy)- |
| | | 9-naphthalen-1-ylmethyl-2,3,4,9- |
| | | tetrahydro-1H-b-carboline |
| 522 | *** | 6-(2-Methyl-3-trifluoromethyl-benzyloxy)- |
| | | 9-naphthalen-2-ylmethyl-2,3,4,9- |
| | | tetrahydro-1H-b-carboline |
| 523 | *** | [6-(2-Methyl-3-trifluoromethyl- |
| | | benzyloxy)-1,2,3,4-tetrahydro-b-carbolin- |
| | | 9-yl]-naphthalen-1-yl-methanone |
| 524 | *** | [6-(2-Methyl-3-trifluoromethyl- |
| | | benzyloxy)-1,2,3,4-tetrahydro-b-carbolin- |
| | | 9-yl]-naphthalen-2-yl-methanone |
| 525 | *** | 6-(3,5-Dimethoxy-benzyloxy)-9-naphthalen- |
| | | 1-ylmethyl-2,3,4,9-tetrahydro-1H-b- |
| | | carboline |
| 526 | *** | [6-(3,5-Dimethoxy-benzyloxy)-1,2,3,4- |
| | | tetrahydro-b-carbolin-9-yl]-naphthalen-1- |
| | | yl-methanone |
| 527 | *** | [6-(3,5-Dimethoxy-benzyloxy)-1,2,3,4- |
| | | tetrahydro-b-carbolin-9-yl]-naphthalen-2- |
| | | yl-methanone |
| 528 | *** | [6-(2-Iodo-benzyloxy)-1,2,3,4-tetrahydro- |
| | | b-carbolin-9-yl]-naphthalen-1-yl- |
| | | methanone |
| 529 | *** | [6-(2-Difluoromethoxy-benzyloxy)-1,2,3,4- |
| | | tetrahydro-b-carbolin-9-yl]-naphthalen-1- |
| | | yl-methanone |
| 530 | *** | [6-(2-Difluoromethoxy-benzyloxy)-1,2,3,4- |
| | | tetrahydro-b-carbolin-9-yl]-naphthalen-2- |
| | | yl-methanone |
| 531 | *** | 4′-(9-Naphthalen-2-ylmethyl-2,3,4,9- |
| | | tetrahydro-1H-b-carbolin-6-yloxymethyl)- |
| | | biphenyl-2-carbonitrile |
| 532 | *** | 4′-[9-(Naphthalene-1-carbonyl)-2,3,4,9- |
| | | tetrahydro-1H-b-carbolin-6-yloxymethyl]- |
| | | biphenyl-2-carbonitrile |
| 533 | *** | 9-Naphthalen-1-ylmethyl-6-(4- |
| | | trifluoromethyl-benzyloxy)-2,3,4,9- |
| | | tetrahydro-1H-b-carboline |
| 534 | *** | 9-Naphthalen-2-ylmethyl-6-(4- |
| | | trifluoromethyl-benzyloxy)-2,3,4,9- |
| | | tetrahydro-1H-b-carboline |
| 535 | *** | 9-Naphthalen-2-ylmethyl-6-(2- |
| | | trifluoromethyl-benzyloxy)-2,3,4,9- |
| | | tetrahydro-1H-b-carboline |
| 536 | *** | Naphthalen-2-yl-[6-(4-trifluoromethyl- |
| | | benzyloxy)-1,2,3,4-tetrahydro-b-carbolin- |
| | | 9-yl]-methanone |
| 537 | *** | 9-Naphthalen-2-ylmethyl-6-(3- |
| | | trifluoromethyl-benzyloxy)-2,3,4,9- |
| | | tetrahydro-1H-b-carboline |
| 196 | *** | Naphthalene-1-carboxylic acid [6-(3,4- |
| | | dichloro-phenyl)-2-piperazin-1-yl- |
| | | pyridin-3-yl]-amide |
| 197 | ** | Naphthalene-1-carboxylic acid [2-(3,4- |
| | | dichloro-phenyl)-4-piperazin-1-yl- |
| | | pyrimidin-5-yl]-amide |

“*” means a Ki > 30 uM
“**” means a Ki from 3 uM to 30 uM
“***” means a Ki < 3 uM

While we have described a number of embodiments of this invention, it is apparent that our basic examples may be altered to provide other embodiments which utilize the compounds and methods of this invention. Therefore, it will be appreciated that the scope of this invention is to be defined by the appended claims rather than by the specific embodiments which have been represented by way of example.

We claim:

1. A BACE inhibitor having the following features:
(a) HB-1;
(b) HPB-4;
and at least one of the following (c) and (d):
(c) HPB-2; and
(d) HPB-3.

2. A BACE inhibitor having the following features:
(a) HB-1;
(b) HPB-4;
(c) HPB-1
and at least one of the following (d) and (e):
(d) HPB-2; and
(e) HPB-3.

3. The BACE inhibitor according to claim 1 or 2, wherein each of the HB-1, HB-2 and HB-3 is independently less than about 3.5 Å in length.

4. The BACE inhibitor according to claim 3, wherein each of HB-1, HB-2 and HB-3 is independently less about 3.0 Å.

5. The BACE inhibitor according to any of claims 1-4, wherein HB-1 is replaced with a electropositive moiety comprising one or more positively charged atoms, wherein said electropositive moiety forms a salt bridge with the carboxylate oxygen atoms of Asp-228 and Asp-32.

6. The BACE inhibitor according to claim 2, wherein the distance between the center of mass of the HPB-1 moiety and the C-β atom of substantially all of Thr-231, Thr-232, Asn-233, Arg-235 and Gln-73 is between about 4.0 Å to about 12 Å.

7. The BACE inhibitor according to claim 6, wherein the distance between the center of mass of the hydrophobic moiety and the C-β atom of substantially all of Thr-231, Thr-232, Asn-233, Arg-235 and Gln-73 is between about 5.0 Å to about 10 Å.

8. The BACE inhibitor according to claim 7, wherein the distance between the center of mass of HPB-1 and the C-β atom of substantially all of Thr-231, Thr-232, Asn-233, Arg-235 and Gln-73 is as follows:
Thr-232—between 5.5 to 6.5 Å;
Thr-232—between 6.0 to 6.7 Å;
Asn-233—between 7.0 to 8.5 Å;
Arg-235—between 8.5 to 10.0 Å; and
Gln-73—between 9.0 to 10.0 Å.

9. The BACE inhibitor according to claim 1, wherein the distance between the center of mass of the HPB-2 moiety and the C-β atom of substantially all of Trp-76, Phe-108, Phe-109, Trp-115 and Ile-102 is between about 3.0 Å to about 8.5 Å.

10. The BACE inhibitor according to claim 9, wherein the distance between the center of mass of the HPB-2 moiety and the C-β atom of substantially all of Trp-76, Phe-108, Phe-109, Trp-115 and Ile-102 is between about 3.5 Å to about 8.0 Å.

11. The BACE inhibitor according to claim 10, wherein the distance between the center of mass of the HPB-2 moiety and the C-β atom of substantially all of Trp-76, Phe-108, Phe-109, Trp-115 and Ile-102 is:
Trp-76—about 8 Å;
Phe-108—about 3.5 Å;
Phe-109—about 6 Å;
Trp-115—about 8 Å; and
Ile-102—about 6 Å.

12. The BACE inhibitor according to claim 1, wherein the distance between the center of mass of the HPB-3 moiety and the C-β atom of substantially all of Asn-37, Ala-39, Val-69, Trp-76, Ile-118 and Arg-128 is between 3.5 Å to 8 Å.

13. The BACE inhibitor according to claim 12, wherein the distance between the center of mass of the HPB-3 moiety and the C-β atom of substantially all of Asn-37, Ala-39, Val-69, Trp-76, Ile-118 and Arg-128 is between 4 Å to 7.5 Å.

14. The BACE inhibitor according to claim 13, wherein the distance between the center of mass of the HPB-3 moiety and the C-β atom of substantially all of Asn-37, Ala-39, Val-69, Trp-76, Ile-118 and Arg-128 is:
Asn-37—between 4.0 Å to 5.0 Å;
Ala-39—about 6 Å;
Val-69—about 6 Å;
Trp-76—about 7.5 Å;
Ile-118—about 6.7 Å; and
Arg-128—about 6 Å.

15. The BACE inhibitor according to claim 1 or 2, wherein HPB-4 is an aromatic stacking moiety that interacts favorably with the phenyl ring of at least two of Tyr-71, Phe-108 and Trp-76.

16. The BACE inhibitor according to claim 15, wherein the HPB-4 moiety interacts with at least two of Tyr-71, Phe-108 and Trp-76 such that the distance between the center of mass of the HPB-4 moiety and the C-β atom of at least two of Tyr-71, Phe-108 and Trp-76 is between 5.5 Å and 8.5 Å.

17. The BACE inhibitor according to claim 16, wherein the HPB-4 moiety interacts with at least two of Tyr-71, Phe-108 and Trp-76 such that the distance between the center of mass of the HPB-4 moiety and the C-β atom of at least two of Tyr-71, Phe-108 and Trp-76 is between 6.0 Å and 8.0 Å.

18. The BACE inhibitor according to claim 17, wherein the HPB-4 moiety interacts with at least two of Tyr-71, Phe-108 and Trp-76 such that the distance between the center of mass of the HPB-4 moiety and the C-β atom of at least two each of Tyr-71, Phe-108 and Trp-76 is as follows:
Tyr-71—about 6.0 Å;
Phe-108—about 5.5 Å; and
Trp-76—about 7 Å.

19. The BACE inhibitor according to claim 18, wherein the HPB-4 moiety interacts with Try-71.

20. The BACE inhibitor according to any one of claim 1 or 2, wherein the distance between the HB-1 moiety and other moieties in the inhibitor, when present, is in the range as set forth below in Table 1:
TABLE 1

| | HB-1^{a} |
|---|---|
| HB-2 | 4.0-5.0 |
| HB-3 | 4.0-5.0 |
| HPB-4 | 5.0-6.0 |
| HPB-1 | 7.0-8.5 |
| HPB-2 | 9.0-11.0 |
| HPB-3 | 8.0-11.0 |

^{a}distances in Angstroms (Å).

21. An enzyme-inhibitor complex, comprising BACE complexed with an inhibitor according to claim 1 or 2.

22. A pharmaceutical composition comprising an inhibitor according to claim 1 or 2, and a pharmaceutically acceptable carrier.

23. A method of inhibiting BACE in a mammal, comprising the step of contacting said mammal with a composition according to claim 22.

24. A method of treating a BACE-mediated disease in a mammal, comprising the step of administering to said mammal a composition according to claim 22.

25. A method of treating Alzheimer's Disease in a mammal, comprising the step of administering to said mammal a composition according to claim 22.

26. A method of inhibiting BACE activity in a mammal, comprising the step of administering to said mammal a compound of formula IA: 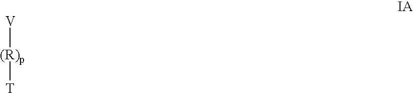
or a pharmaceutically acceptable salt thereof, wherein:
V is a 3-4 membered acyclic group or a 5-7 membered, fully or partially saturated cyclic group;
wherein V comprises a first moiety selected from NH, CH—OH, or a CH—NH₂, and a second moiety selected from carbon, CH, or N;
wherein said first moiety and said second moiety in V are non-adjacent; and
V is attached to R through said second moiety;
wherein V is optionally substituted with R^{10;}
R is a suitable linker;
p is 0 or 1;
R¹⁰ is P1-R1-P2-R2-W;
T is a five to eleven membered monocyclic or bicyclic, aromatic or non-aromatic ring having zero to three heteroatoms independently selected from O, S, N or NH, wherein T has at least one R¹⁰ substituent and up to three more substituents selected from R¹⁰ or J;
J is halogen, —R′, —OR′, —NO₂, —CN, —CF₃, —OCF₃, oxo, 1,2-methylenedioxy, —N(R′)₂, —SR′, —S(O)R′, —S(O)N(R′)₂, —SO₂R′, —C(O)R′, —CO₂R′, —C(O)N(R′)₂, —N(R′)C(O)R′, —N(R′)C(O)OR′, —N(R′)C(O)N(R′)₂, or —OC(O)N(R′)₂, wherein R′ is independently selected from hydrogen, aliphatic, heterocyclyl, heterocycly-alkyl, aryl, aralkyl, heteroaryl, or heteroaralkyl;
wherein R1 is optionally substituted with up to 3 substituents selected independently from —R¹¹, —OR¹¹, —N₂, —CN, —CF₃, —OCF₃, oxo, 1,2-methylenedioxy, —N(R¹¹)₂, —SR¹¹, —S(O)R11, S(O)N(R¹¹)₂, —SO₂R¹¹, —C(O)R¹¹ , —CO₂R¹¹, —C(O)N(R¹¹)₂, —N(R¹¹)C(O)R¹¹, —N(R¹¹)C(O)OR¹¹, —N(R¹¹)C(O)N(R¹¹)₂, or —OC(O)N(R¹¹)₂,;
R¹¹ is hydrogen, (C₁-C₆)-alkyl, (C₂-C₆)-alkenyl or alkynyl, or (C₃-C₆)cycloalkyl;
P1 and P2 each are independently:
absent; or
aliphatic;
R1 and R2 each are independently:
absent; or
R;
W is five to eleven membered monocyclic or bicyclic, aromatic or non-aromatic ring having zero to three heteroatoms independently selected from O, S, N, or NH, wherein W has up to 3 J substituents.

27. The method according to claim 26, wherein R is —CH₂—, —O—, —S—, —SO—, —SO₂—, —NR′—, —C(O)O—, —OC(O)—, —C(O)NR′—, —NR′C(O)—, —O—, —OC(O)NR′—, —NR′C(O)NR′—, —NR′C(O)O—, —SO—NR′, —NR′SO—, —NR′SO₂—, —SO₂NR′—, —CHOR′—, —CHNR′—, or —C(O)—.

28. The method according to claim 26, wherein
R¹⁰ is P1-R1-P2-R2-W:
wherein one of P1 and P2 is absent and the other of P1 and P2 is aliphatic, and/or one of R1 and R2 is absent and the other of R1 and R2 is R.

29. The method according to claim 26, wherein W is a five to seven membered monocyclic, aromatic or non-aromatic ring having zero to three heteroatoms independently selected from O, S, N, or NH, wherein W has up to 3 substituents independently selected from J.

30. The method according to claim 29, wherein W is selected from 2-furanyl, 3-furanyl, 3-furazanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 1-pyrrolyl, 2-pyrrolyl, 3-pyrrolyl, 1-pyrazolyl, 2-pyrazolyl, 3-pyrazolyl, 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, or 3-thienyl.

31. The method according to claim 26, wherein W is a five to six membered monocyclic, non-aromatic ring having one to three heteroatoms independently selected from O, S, N, or NH, wherein W has up to 3 substituents independently selected from J.

32. The method according to claim 31, wherein W is selected from 2-tetrahydrofuranyl, 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, or N-substituted diazolonyl.

33. The method according to claim 26, wherein W is a five to seven membered monocyclic, aromatic or non-aromatic ring having zero heteroatoms independently selected from O, S, N, or NH, wherein W has up to 3 substituents independently selected from J.

34. The method according to claim 33, wherein W is phenyl optionally substituted with up to 3 substituents independently selected from J.

35. The method according to claim 26, wherein V is selected from IA-1 through IA-9 shown below:

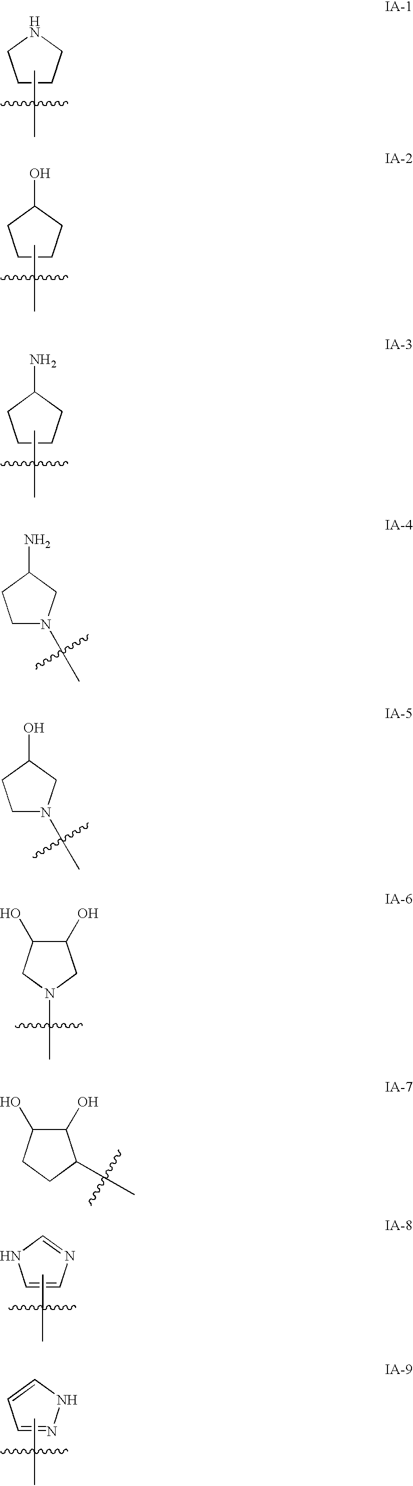

36. The method according to claim 35, wherein V is selected from IA-1, IA-8, or IA-9.

37. The method according to claim 26, wherein V is selected from formula IB-1 to formula IB-6 shown below:

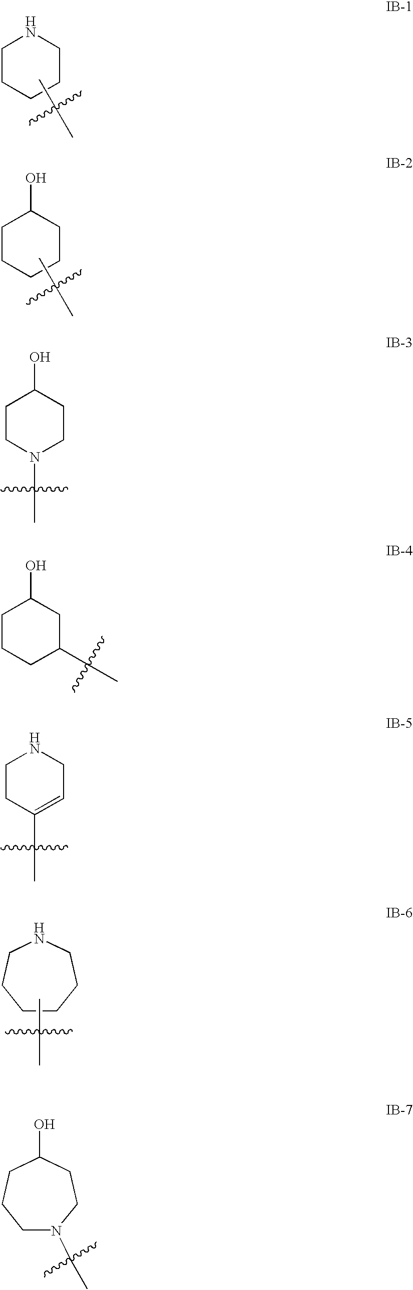

38. The method according to claim 37, wherein V is IB-1 or IB-5.

39. The method according to claim 38, wherein V is IB-5.

40. A method of inhibiting BACE activity in a mammal, comprising the step of administering to said mammal a compound of formula IAB: 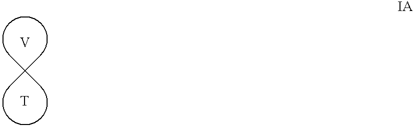
wherein:
V is selected from IA1, IB1, IB2, IB4, IB5, or IB6;
T is a five to eleven membered monocyclic or bicyclic, aromatic or non-aromatic ring having zero to three heteroatoms independently selected from O, S, N or NH, wherein T has at least one R¹⁰ substituent and up to three more substituents selected from R¹⁰ or J;
T and V share a ring atom;
J is halogen, —R′ , —OR′, —NO₂, —CN, —CF₃, —OCF₃, oxo, 1,2-methylenedioxy, —N(R′)₂, —SR′, —S(O)R′, —S(O)N(R′)₂, —SO₂R′, —C(O)R′, —CO₂R′, —C(O)N(R′)₂, —N(R′)C(O)R′, —N(R′)C(O)OR′, —N(R′)C(O)N(R′)₂, or —OC(O)N(R′)₂, wherein R′ is independently selected from hydrogen, aliphatic, heterocyclyl, heterocycly-alkyl, aryl, aralkyl, heteroaryl, or heteroaralkyl;
wherein R′ is optionally substituted with up to 3 substituents selected independently from —R¹¹, —OR¹¹, —NO₂, —CN, —CF₃, —OCF₃, oxo, 1,2-methylenedioxy, —N(R¹¹)₂, —SR¹¹, —S(O)R¹¹, —S(O)N(R¹¹)₂, —SO₂R¹¹, —C(O)R¹¹, —CO₂R¹¹, —C(O)N(R¹¹)₂, —N(R¹¹)C(O)R′, —N(R¹¹)C(O)OR¹¹, —N(R¹¹)C(O)N(R¹¹)₂, or —OC(O)N(R¹¹)₂,;
R¹¹ is hydrogen, (C₁-C₆)-alkyl, (C₂-C₆)-alkenyl or alkynyl, or (C₃-C₆)cycloalkyl;
R¹⁰ is P1-R1-P2-R2-W;
P1 and P2 each are independently:
absent; or
aliphatic;
R1 and R2 each are independently:
absent; or
R;
R is a suitable linker;
W is five to eleven membered monocyclic or bicyclic, aromatic or non-aromatic ring having zero to three heteroatoms independently selected from O, S, N, or NH, wherein W has up to 3 J substituents.

41. The method according to claim 26, wherein said compound of formula (IA) is selected from: 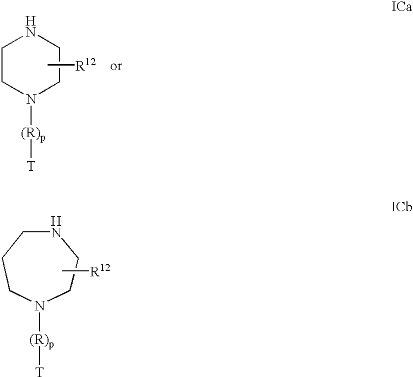
or a pharmaceutically acceptable salt thereof, wherein:
R¹² is absent or R¹⁰;
R¹⁰, R, p and T are as defined in claim 26.

42. The method according to claim 41, wherein said compound is ICa, wherein R¹² is absent.

43. A method of inhibiting BACE activity in a mammal, comprising the step of administering to said mammal a compound of formula ID: 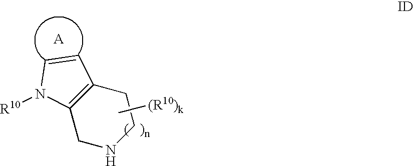
or a pharmaceutically acceptable salt thereof, wherein:
A is a five or six membered aryl ring having zero to two heteroatoms independently selected from nitrogen, oxygen or sulfur, wherein:
A has at least one R¹⁰ substituent and up to three more substituents selected from R¹⁰ or J;
k is 0 or 1;
n is 0-2;
J is halogen, —R′, —OR′, —NO₂, —CN, —CF₃, —OCF₃, oxo, 1,2-methylenedioxy, —N(R′)₂, —SR′, —S(O)R′, —S(O)N(R′)₂, —SO₂R′, —C(O)R′, —CO₂R′, —C(O)N(R′)₂, —N(R′)C(O)R′, —N(R′)C(O)OR′, —N(R′)C(O)N(R′)₂, or —OC(O)N(R′)₂, wherein R′ is independently selected from hydrogen, aliphatic, heterocyclyl, heterocycly-alkyl, aryl, aralkyl, heteroaryl, or heteroaralkyl;
wherein R′ is optionally substituted with up to 3 substituents selected independently from —R¹¹, —OR¹¹, —NO₂, —CN, —CF₃, —OCF₃, oxo, 1, 2-methylenedioxy, —N(R¹¹)₂, —SR¹¹, —S(O)R₁₁ S(O)N(R¹¹)₂, —SO₂R¹¹, —C(O)R¹¹, —CO₂R¹¹ C(O)N(R¹¹)₂, —N(R¹¹)C(O)R′, —N(R¹¹)C(O)OR¹¹, —N(R¹¹) C(O)N(R¹¹)₂, or —OC(O)N(R¹¹)₂,;
R¹¹ is hydrogen, (C₁-C₆)-alkyl, (C₂-C₆)-alkenyl or alkynyl, or (C₃-C₆)cycloalkyl;
R¹⁰ is P1-R1-P2-R2-W;
P1 and P2 each are independently:
absent; or
aliphatic;
R1 and R2 each are independently:
absent; or
R;
R is a suitable linker;
W is a five to eleven membered monocyclic or bicyclic, aromatic or non-aromatic ring having zero to three heteroatoms independently selected from O, S, N, or NH, wherein W has up to 3 substituents independently selected from J.

44. The method according to claim 43, wherein said compound is compound of formula ID-1 or formula ID2: 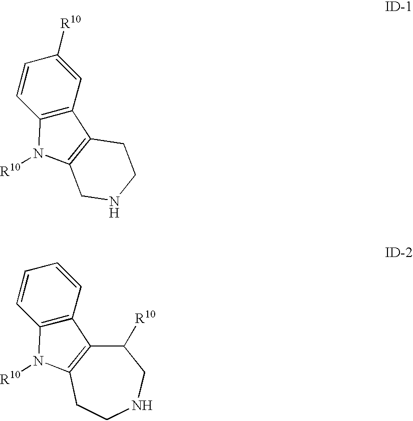
wherein R¹⁰ is as defined in claim 43.

45. A method of inhibiting BACE activity in a mammal, comprising the step of administering to said mammal a compound of formula IE: 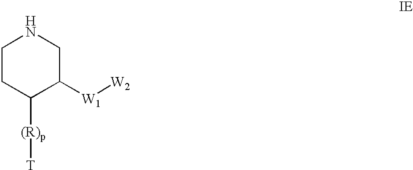
wherein:
W₁ is —NH—, —CH₂—NH—, —C(O)—NH—, or —C(O)—O—;
W₂ is P1-R1-P2-R2-W;
P1 and P2 each are independently:
absent; or
aliphatic;
R1 and R2 each are independently:
absent; or
R;
W is five to eleven membered monocyclic or bicyclic, aromatic or non-aromatic ring having zero to three heteroatoms independently selected from O, S, N, or NH, wherein W has up to 3 substituents independently selected from J;
R is —CH₂—, —O—, —S—, —SO—, —SO₂—, —NR′—, —C(O)O—, —OC(O)—, —C(O)NR′—, —NR′C(O)—, —O—, —OC(O)NR′—, —NR′C(O)NR′—, —NR′C(O)O—, —SO—NR′, —NR′SO—, —NR′SO₂—, —SO₂NR′—, —CHOR′—, —CHNR′—, or —C(O)—;
J is halogen, —R′, —OR′, —NO₂, —CN, —CF₃, —OCF₃, oxo, 1,2-methylenedioxy, —N(R′)₂, —SR′, —S(O)R′, —S(O)N(R′)₂, —SO₂R′, —C(O)R′, —CO₂R′, —C(O)N(R′)₂, —N(R′)C(O)R′, —N(R′)C(O)OR′, —N(R′)C(O)N(R′)₂, or —OC(O)N(R′)₂, wherein R′ is independently selected from hydrogen, aliphatic, heterocyclyl, heterocycly-alkyl, aryl, aralkyl, heteroaryl, or heteroaralkyl;
wherein R′ is optionally substituted with up to 3 substituents selected independently from —R¹¹, —OR¹, —NO₂, —CN, —CF₃, —OCF₃, oxo, 1,2-methylenedioxy, —N(R¹¹)₂, —SR¹¹, —S(O)R11, —S(O)N(R¹¹)₂, —SO₂R¹¹, —C(O)R11, —CO₂R¹¹, —C(O)N(R¹¹)₂, —N(R¹¹)C(O)R′, —N(R¹¹)C(O)OR¹¹, —N(R¹¹)C(O)N(R¹¹)₂, or —OC(O)N(R¹¹)₂,;
R¹¹ is hydrogen, (C₁-C₆)-alkyl, (C₂-C₆)-alkenyl or alkynyl, or (C₃-C₆)cycloalkyl;
T is a five to eleven membered monocyclic or bicyclic, aromatic or non-aromatic ring having zero to three heteroatoms independently selected from O, N or NH, wherein T has at least one R¹⁰ substituent and up to three more substituents selected from R¹⁰ or J;

46. The method according to claim 45, wherein W₁ is —NH—, —CH₂—NH— or —C(O)—NH—.

47. The method according to claim 46, wherein W₁ is —NH—.

48. The method according to claim 47, wherein:
p is 0; and
T is selected from phenyl or naphthyl, wherein T has at least one R¹⁰ substituent and up to three more substituents selected from R¹⁰ or J.

49. A method of inhibiting BACE activity in a mammal, comprising the step of contacting said mammal with a compound selected from Tables IA-ID.

50. The method according to claim 49, wherein said compound is selected from Table IB or IC.

51. A compound of formula II: 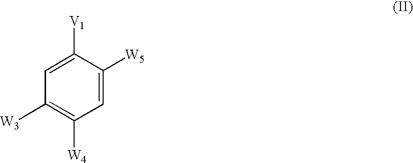
wherein:
V₁ is selected from: 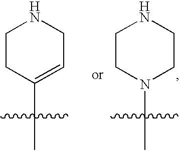
wherein V₁ is optionally substituted with R^{10;}
W₃ is hydrogen or 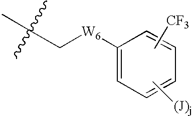
wherein:
W6 is selected from —O—, —S—, or —NH—;
j is 0 to 3;
W₄ is hydrogen or a 5-11 membered monocyclic or bicyclic aromatic ring having 0-3 heteroatoms independently selected from O, S, N, or NH, wherein W₄ has up to 3 J substituents;
W₅ is hydrogen or R^{10;}
provided that at least two or W3, W₄, and W₅ are simultaneously non-hydrogen;
R¹⁰ is P1-R1-P2-R2-W;
J is halogen, —R′, —OR′, —NO₂, —CN, —CF₃, —OCF₃, oxo, 1,2-methylenedioxy, —N(R′)₂, —SR′, —S(O)R′, —S(O)N(R′)₂, —SO₂R′, —C(O)R′, —CO₂R′, —C(O)N(R′)₂, —N(R′)C(O)R′, —N(R′)C(O)OR′, —N(R′)C(O)N(R′)₂, or —OC(O)N(R′)₂, wherein R′ is independently selected from hydrogen, aliphatic, heterocyclyl, heterocycly-alkyl, aryl, aralkyl, heteroaryl, or heteroaralkyl;
wherein R′ is optionally substituted with up to 3 substituents selected independently from —R¹¹, —OR¹¹, —NO₂, —CN, —CF₃, —OCF₃, oxo, 1,2-methylenedioxy, —N(R¹¹)₂, —SR¹¹, —S(O)R¹¹, —S(O)N(R¹¹)₂, —SO₂R¹¹, —C(O)R11, —CO₂R , —C(O)N(R¹¹)₂, —N(R¹¹)C(O)R′, —N(R¹¹)C(O)OR¹¹, —N(R¹¹)C(O)N(R¹¹)₂, or —OC(O)N(R¹¹)₂,;
R¹¹ is hydrogen, (C₁-C₆)-alkyl, (C₂-C₆)-alkenyl or alkynyl, or (C₃-C₆)cycloalkyl;
P1 and P2 each are independently:
absent; or
aliphatic;
R1 and R2 each are independently:
absent; or
R;
R is a suitable linker; and
W is five to eleven membered monocyclic or bicyclic, aromatic or non-aromatic ring having zero to three heteroatoms independently selected from O, S, N, or NH, wherein W has up to 3 J substituents.

52. The compound according to claim 51, wherein, j is selected from 1, 2 or 3.

53. The compound according to claim 51, wherein W₃ is 2-trifluoromethyl-phenoxymethyl.

54. The compound according to claim 51, wherein VI is unsubstituted 3,4-didehydropiperidyl.

55. The compound according to claim 51, wherein VI is unsubstituted piperazyl.

56. The compound according to claim 51, W or W₄ is independently phenyl or a five to seven membered monocyclic, aromatic ring having 1-3 heteroatoms independently selected from O, S, N, or NH, wherein W or W₄ has up to 3 substituents independently selected from J.

57. The compound according to claim 56, wherein W or W₄ is selected from 2-furanyl, 3-furanyl, 3-furazanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 1-pyrrolyl, 2-pyrrolyl, 3-pyrrolyl, 1-pyrazolyl, 2-pyrazolyl, 3-pyrazolyl, 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, or 3-thienyl, wherein W or W₄ has up to 3 J substituents.

58. The compound according to claim 58, wherein W or W₄ is an eight to eleven membered bicyclic ring, wherein either or both rings is aromatic, and either or both rings has zero to three heteroatoms independently selected from O, S, N, or NH, wherein W or W₄ has up to 3 substituents independently selected from J.

59. The compound according to claim 59, wherein W or W₄ is selected from naphthyl, 3-1H-benzimidazol-2-one, (1-substituted)-2-oxo-benzimidazol-3-yl, 1-phthalimidinyl, benzoxanyl, benzopyrrolidinyl, benzopiperidinyl, benzoxolanyl, benzothiolanyl, benzothianyl, indolinyl, chromanyl, phenanthridinyl, tetrahydroquinolinyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, indolyl, isoindolyl, acridinyl, benzoisoxazolyl, tetrahydroquinolinyl, tetrahydroisoquinolinyl, or pyrido[3,4-d]pyrimidiny, wherein W or W₄ has up to 3 J substituents.

60. The compound according to claim 56, wherein W₄ is phenyl or 5-hydroxyphenyl.

61. The compound according to claim 51, wherein W₅ is P1-R1-W or R1-P2-W.

62. The compound according to claim 61, wherein each of P1 and P2 is independently (C₁-C₆)-alkyl, and R1 is R.

63. The compound according to claim 62, wherein R is selected from —CH₂—, —O—, —S—, —SO—, —SO₂—, —NR′—, —C(O)O—, —OC(O)—, —C(O)NR′—, —NR′C(O)—, —O—, —OC(O)NR′—, —NR′C(O)O—, —NR′C(O)NR′—, —NR′C(O)O—, —SO—NR′, —NR′SO—, —NR′SO₂—, —SO₂NR′—, —CHOR′—, —CHNR′—, or —C(O)—.

64. The compound according to claim 61, wherein:
each of P1 and P2 is methylene;
R1 is —O—, —NH—C(O)—, —C(O)—NH—, or —NH—; and
W is selected from phenyl, 4-hydroxyphenyl, 1-napthyl, 2-napthyl, isoquinolinyl, quinolinyl, or 2-trifluoromethylphenyl.

64. The compound according to claim 51, wherein J is independently selected from halogen, —R′, —OR′, —NO₂, —CN, —CF₃, —OCF₃, oxo, 1,2-methylenedioxy, —N(R′)₂, —SR′, —S(O)R′, —S(O)N(R′)₂, —SO₂R′, —C(O)R′, —CO₂R′ or —C(O)N(R′)₂, wherein R′ is independently selected from hydrogen or (C₁-C₆)-alkyl.

65. The compound according to claim 64, wherein in W₃, j is 1-3.

66. A composition comprising a compound according to claim 51, and a pharmaceutically acceptable carrier.

67. The composition according to claim 66, wherein said compound is selected from Tables 1A-1D.

68. A method of inhibiting BACE activity in a mammal comprising the step of contacting said mammal with a compound according to claim 51.

69. A method of treating a BACE-mediated disease in a mammal, comprising the step of administering to said mammal a composition according to claim 66.

70. The method according to claim 69, wherein said disease is Alzheimer's Disease, MCI (“mild cognitive impairment”), Down's syndrome, hereditary cerebral hemorrhage, cerebral amyloid angiopathy, dementia.
